(19) United States
(12) Patent Application Publication
(10) Pub. No.: US 2021/0061809 A1
(43) Pub. Date: Mar. 4, 2021

(54) TRIAZOLOPYRIMIDINES AS A2A / A2B INHIBITORS

(71) Applicant: Incyte Corporation, Wilmington, DE (US)

(72) Inventors: Heeoon Han, Elkins Park, PA (US); Le Zhao, Wilmington, DE (US); Wenqing Yao, Chadds Ford, PA (US); Xiaozhao Wang, Mt. Laurel, NJ (US)

(21) Appl. No.: 17/002,045

(22) Filed: Aug. 25, 2020

Related U.S. Application Data
(60) Provisional application No. 62/891,685, filed on Aug. 26, 2019.

(51) Int. Cl. C07D 487/04

(52) U.S. Cl. CPC C07D 487/04

(57) ABSTRACT

This application relates to compounds of Formula (I):

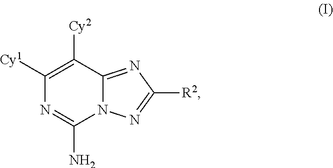

or pharmaceutically acceptable salts or stereoisomers thereof, which modulate the activity of adenosine receptors, such as subtypes A2A and A2B receptors, and are useful in the treatment of diseases related to the activity of adenosine receptors including, for example, cancer, inflammatory diseases, cardiovascular diseases, and neurodegenerative diseases.

### CROSS-REFERENCE TO RELATED APPLICATIONS

This application claims the benefit of U.S. Provisional Application Ser. No. 62/891,685, filed Aug. 26, 2019, the disclosure of which is incorporated herein by reference in its entirety.

### TECHNICAL FIELD

The present invention provides triazolopyrimidine compounds that modulate the activity of adenosine receptors, such as subtypes A2A and A2B, and are useful in the treatment of diseases related to the activity of adenosine receptors including, for example, cancer, inflammatory diseases, cardiovascular diseases, and neurodegenerative diseases.

### BACKGROUND

Adenosine is an extracellular signaling molecule that can modulate immune responses through many immune cell types. Adenosine was first recognized as a physiologic regulator of coronary vascular tone by Drury and Szent-Gyorgyu (Sachdeva, S. and Gupta, M. Saudi Pharmaceutical Journal, 2013, 21, 245-253), however it was not until 1970 that Sattin and Rall showed that adenosine regulates cell function via occupancy of specific receptors on the cell surface (Sattin, A., and Rall, T. W., 1970. Mol. Pharmacol. 6, 13-23; Hasko', G., at al., 2007, Pharmacol. Ther. 113, 264-275).

Adenosine plays a vital role in various other physiological functions. It is involved in the synthesis of nucleic acids, when linked to three phosphate groups; it forms ATP, the integral component of the cellular energy system. Adenosine can be generated by the enzymatic breakdown of extracellular ATP, or can be also released from injured neurons and glial cells by passing the damaged plasma membrane (Tautenhahn, M. et al. Neuropharmacology, 2012, 62, 1756-1766). Adenosine produces various pharmacological effects, both in periphery and in the central nervous system, through an action on specific receptors localized on cell membranes (Matsumoto, T. et al. Pharmacol. Res., 2012, 65, 81-90). Alternative pathways for extracellular adenosine generation have been described. These pathways include the production of adenosine from nicotinamide dinucleotide (NAD) instead of ATP by the concerted action of CD38, CD203a and CD73. CD73-independent production of adenosine can also occur by other phosphates such as alkaline phosphatase or prostate-specific phosphatase.

There are four known subtypes of adenosine receptor in humans including A1, A2A, A2B, and A3 receptors. A1 and A2A are high affinity receptors, whereas A2B and A3 are low affinity receptors. Adenosine and its agonists can act via one or more of these receptors and can modulate the activity of adenylate cyclase, the enzyme responsible for increasing cyclic AMP (cAMP). The different receptors have differential stimulatory and inhibitory effects on this enzyme. Increased intracellular concentrations of cAMP can suppress the activity of immune and inflammatory cells (Livingston, M. et al., Inflamm. Res., 2004, 53, 171-178).

The A2A adenosine receptor can signal in the periphery and the CNS, with agonists explored as anti-inflammatory drugs and antagonists explored for neurodegenerative diseases (Carlsson, J. et al., J. Med. Chem., 2010, 53, 3748-3755). In most cell types the A2A subtype inhibits intracellular calcium levels whereas the A2B potentiates them. The A2A receptor generally appears to inhibit inflammatory response from immune cells (Borrmann, T. et al., J. Med. Chem., 2009, 52(13), 3994-4006).

A2B receptors are highly expressed in the gastrointestinal tract, bladder, lung and on mast cells (Antonioli, L. et al., Nature Reviews Cancer, 2013, 13, 842-857). The A2B receptor, although structurally closely related to the A2A receptor and able to activate adenylate cyclase is functionally different. It has been postulated that this subtype may utilize signal transduction systems other than adenylate cyclase (Livingston, M. et al., Inflamm. Res., 2004, 53, 171-178). Among all the adenosine receptors, the A2B adenosine receptor is a low affinity receptor that is thought to remain silent under physiological conditions and to be activated in consequence of increased extracellular adenosine levels (Ryzhov, S. et al. Neoplasia, 2008, 10, 987-995). Activation of A2B adenosine receptor can stimulate adenylate cyclase and phospholipase C through activation of Gs and Gq proteins, respectively. Coupling to mitogen activated protein kinases has also been described (Borrmann, T. et al., J. Med. Chem., 2009, 52(13), 3994-4006).

In the immune system, engagement of adenosine signaling can be a critical regulatory mechanism that protects tissues against excessive immune reactions.

Adenosine can negatively modulate immune responses through many immune cell types, including T-cells, natural-killer cells, macrophages, dendritic cells, mast cells and myeloid-derived suppressor cells (Allard, B. et al. Current Opinion in Pharmacology, 2016, 29, 7-16).

In tumors, this pathway is hijacked by tumor micro-environments and sabotages the antitumor capacity of immune system, promoting cancer progression. In the tumor micro-environment, adenosine was mainly generated from extracellular ATP by CD39 and CD73. Multiple cell types can generate adenosine by expressing CD39 and CD73. This is the case for tumor cells, T-effector cells, T-regulatory cells, tumor associated macrophages, myeloid derived suppressive cells (MDSCs), endothelial cells, cancer-associated fibroblast (CAFs) and mesenchymal stromal/stem cells (MSCs). Hypoxia, inflammation and other immune-suppressive signaling in tumor micro-environment can induce expression of CD39, CD73 and subsequent adenosine production. As a result, adenosine level in solid tumors is unusually high compared to normal physiological conditions.

A2A are mostly expressed on lymphoid-derived cells, including T-effector cells, T regulatory cells and nature killing cells. Blocking A2A receptor can prevent downstream immunosuppressive signals that temporarily inactivate T cells. A2B receptors are mainly expressed on monocyte-derived cells including dendritic cells, tumor-associated macrophages, myeloid derived suppressive cells (MDSCs), and mesenchymal stromal/stem cells (MSCs). Blocking A2B receptor in preclinical models can suppress tumor growth, block metastasis, and increase the presentation of tumor antigens.

In terms of safety profile of ADORA2A/ADORA2B (A2A/A2B) blockage, the A2A and A2B receptor knockout mice are all viable, showing no growth abnormalities and are fertile (Allard, B. et al. Current Opinion in Pharmacology, 2016, 29, 7-16). A2A KO mice displayed increased levels of pro-inflammatory cytokines only upon challenge with LPS and no evidence of inflammation at baseline (Antonioli, L. et al., Nature Reviews Cancer, 2013, 13, 842-857). A2B KO mice exhibited normal platelet, red blood, and white cell counts but increased inflammation at baseline (TNF-alpha, IL-6) in naive A2B KO mice (Antonioli, L. et al., Nature Reviews Cancer, 2013, 13, 842-857). Exaggerated production of TNF-alpha and IL-6 was detected following LPS treatment. A2B KO mice also exhibited increased vascular adhesion molecules that mediate inflammation as well leukocyte adhesion/rolling; enhanced mast-cell activation; increased sensitivity to IgE-mediated anaphylaxis and increased vascular leakage and neutrophil influx under hypoxia (Antonioli, L. et al., Nature Reviews Cancer, 2013, 13, 842-857).

In summary, there is a need to develop new adenosine receptor selective ligands, such as for subtypes A2A and A2B, for the treatment of diseases such as cancer, inflammatory diseases, cardiovascular diseases and neurodegenerative diseases. This application is directed to this need and others.

### SUMMARY

The present invention relates to, inter alia, compounds of Formula (I):

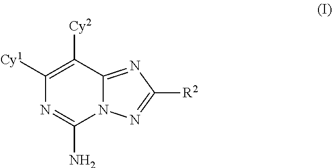

or pharmaceutically acceptable salts thereof, wherein constituent members are defined herein.

The present invention further provides pharmaceutical compositions comprising a compound of Formula (I), or a pharmaceutically acceptable salt thereof, and a pharmaceutically acceptable carrier.

The present invention further provides methods of inhibiting an activity of an adenosine receptor, comprising contacting the receptor with a compound of Formula (I), or a pharmaceutically acceptable salt thereof.

The present invention further provides methods of treating a disease or a disorder associated with abnormal expression of adenosine receptors, comprising administering to said patient a therapeutically effective amount of a compound of Formula (I), or a pharmaceutically acceptable salt thereof.

The present invention further provides a compound of Formula (I), or a pharmaceutically acceptable salt thereof, for use in any of the methods described herein.

The present invention further provides use of a compound of Formula (I), or a pharmaceutically acceptable salt thereof, for the preparation of a medicament for use in any of the methods described herein.

### DETAILED DESCRIPTION

### Compounds

The present invention relates to, inter alia, compounds of Formula (I):

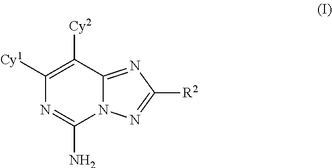

or pharmaceutically acceptable salts thereof; wherein:

R² is selected from L-W and L-W′-Z;

wherein L is selected from C₁₋₃ alkyl, —C₁₋₃ alkyl-O—, —O—C₁₋₃ alkyl-, —C₁₋₃ alkyl-NH—, —NH—C₁₋₃ alkyl-, —NH—C₁₋₃ alkyl-NH—, and —N(C₁₋₃ alkyl)-;

wherein W is a 5-6 membered heteroaryl optionally substituted with 1, 2 or 3 groups each independently selected from cyano, halogen, and C₁₋₃ alkyl;

wherein W′ is phenyl or 5-6 membered heteroaryl, wherein each phenyl or 5-6 membered heteroaryl of W′ is optionally substituted with 1, 2, or 3 groups each independently selected from cyano, halogen, and C₁₋₃ alkyl; and wherein Z is a phenyl or 5-6 membered heteroaryl, wherein each phenyl or 5-6 membered heteroaryl of Z is optionally substituted with 1, 2, or 3 groups each independently selected from cyano, halogen, C₁₋₃ alkyl, amine, and C₁₋₃ alkoxy;

Cy¹ is selected from cyanophenyl and cyanofluorophenyl; and

Cy² is a 5-6 membered heteroaryl optionally substituted with 1, 2 or 3 groups each independently selected from C₁₋₃ alkyl, C₁₋₃ alkoxy, NH₂, NH(C₁₋₃ alkyl) and N(C₁₋₃ alkyl)₂, and wherein a ring-forming carbon atom of Cy² is optionally substituted by oxo.

In some embodiments, R² is L-W.

In some embodiments, R² is L-W′-Z.

In some embodiments, L is selected from —CH₂—, —NH—CH₂—, —O—CH₂—, —NH—, —CH₂—O—, —NH—CH(CH₃)—, and —NH—C(CH₃)₂—

In some embodiments, W is pyridinyl, which is optionally substituted by 1 or 2 groups each independently selected from methyl, fluoro, and cyano,

In some embodiments, W′ is selected from tetrazolyl and phenyl, wherein phenyl is optionally substituted with fluoro.

In some embodiments, Z is selected from phenyl, pyridinyl, pyrazolyl, thiazolyl, pyrimidinyl, and pyrazinyl, each of which is optionally substituted with 1 or 2 groups independently selected from cyano, halogen, methyl, amino, and methoxy.

In some embodiments, Cy¹ is 3-cyanophenyl, optionally substituted with fluoro.

In some embodiments, Cy² is selected from pyrimidinyl, 1-methyl-6-oxo-1,6-dihydropyridinyl, 1-methyl-6-oxo-1,6-dihydropyridazinyl, pyridinyl, dimethylaminopyridinyl, aminopyridinyl, 1-ethyl-6-oxo-1,6-dihydropyridinyl, methylpyridinyl, dimethylpyridinyl, and methoxymethylpyridinyl.

In some embodiments, the compound of Formula (I) is selected from:
3-(5-amino-2-((5-(3-aminophenyl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(6-methylpyridin-2-yl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(6-methoxypyridin-2-yl)-1H-tetrazol-1-yl)methyl)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyridin-2-yl)-1H-tetrazol-1-yl)methyl)-[1,2,4]triazolo[1,5-c]pyrimidin-7-yl)benzonitrile;
3-(2-((5-(1H-pyrazol-1-yl)-1H-tetrazol-1-yl)methyl)-5-amino-8-(1-methyl-6-oxo-1,6-dihydropyridin-3-yl)-[1,2,4]triazolo[1,5-c]pyrimidin-7-yl)benzonitrile;
3-(5-amino-8-(1-methyl-6-oxo-1,6-dihydropyridin-3-yl)-2-((5-(thiazol-4-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyrimidin-2-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyrazin-2-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridazin-3-yl)-2-((5-(pyridin-2-yl)-1H-tetrazol-1-yl)methyl)-[1,2,4]triazolo[1,5-c]pyrimidin-7-yl)benzonitrile;
3-(5-amino-2-((5-(pyridin-2-yl)-1H-tetrazol-1-yl)methyl)-8-(pyridin-4-yl)-[1,2,4]triazolo[1,5-c]pyrimidin-7-yl)benzonitrile;
3-(5-amino-8-(2-(dimethylamino)pyridin-4-yl)-2-((5-(pyridin-2-yl)-1H-tetrazol-1-yl)methyl)-[1,2,4]triazolo[1,5-c]pyrimidin-7-yl)benzonitrile;
3-(5-amino-8-(2-aminopyridin-4-yl)-2-((5-(pyridin-2-yl)-1H-tetrazol-1-yl)methyl)-[1,2,4]triazolo[1,5-c]pyrimidin-7-yl)benzonitrile;
3-(5-amino-2-(2-fluoro-6-(pyridin-4-yl)benzyl)-8-(pyridin-4-yl)-[1,2,4]triazolo[1,5-c]pyrimidin-7-yl)benzonitrile;
3-(5-amino-8-(2-aminopyridin-4-yl)-2-(2-(2-aminopyridin-4-yl)-6-fluorobenzyl)-[1,2,4]triazolo[1,5-c]pyrimidin-7-yl)benzonitrile;
3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)benzonitrile;
3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridin-3-yl)-2-((3-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridazin-3-yl)-2-((3-methylpyridin-2-yl)methoxy)-[1,2,4]triazolo[1,5-c]pyrimidin-7-yl)benzonitrile;
3-(5-amino-2-((3-methylpyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((3-methylpyridin-2-yl)methoxy)-[1,2,4]triazolo[1,5-c]pyrimidin-7-yl)benzonitrile;
3-(5-amino-8-(1-ethyl-6-oxo-1,6-dihydropyridin-3-yl)-2-((3-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(2-methoxy-6-methylpyridin-4-yl)-2-(((3-methylpyridin-2-yl)methyl)amino)-[1,2,4]triazolo[1,5-c]pyrimidin-7-yl)-2-fluorobenzonitrile;
3-(5-amino-8-(1-methyl-6-oxo-1,6-dihydropyridazin-3-yl)-2-(pyridin-2-ylmethyl)-[1,2,4]triazolo[1,5-c]pyrimidin-7-yl)benzonitrile;
3-(5-amino-2-((3-fluoropyridin-2-yl)methoxy)-8-(pyrimidin-4-yl)-[1,2,4]triazolo[1,5-c]pyrimidin-7-yl)benzonitrile;
3-(5-amino-2-((3-fluoropyridin-2-yl)methoxy)-8-(1-methyl-6-oxo-1,6-dihydropyridin-3-yl)-[1,2,4]triazolo[1,5-c]pyrimidin-7-yl)benzonitrile;
3-(5-amino-2-((3-fluoropyridin-2-yl)methoxy)-8-(1-methyl-6-oxo-1,6-dihydropyridazin-3-yl)-[1,2,4]triazolo[1,5-c]pyrimidin-7-yl)benzonitrile;
3-(5-amino-2-((3-fluoropyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((3-fluoropyridin-2-yl)methoxy)-[1,2,4]triazolo[1,5-c]pyrimidin-7-yl)benzonitrile;
3-(5-amino-2-((3-fluoropyridin-2-yl)methoxy)-8-(2-methoxy-6-methylpyridin-4-yl)-[1,2,4]triazolo[1,5-c]pyrimidin-7-yl)benzonitrile;
3-(5-amino-2-(pyridin-2-ylamino)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-(pyridin-2-ylamino)-[1,2,4]triazolo[1,5-c]pyrimidin-7-yl)benzonitrile;
3-(5-amino-8-(1-methyl-6-oxo-1,6-dihydropyridazin-3-yl)-2-(pyridin-2-ylamino)-[1,2,4]triazolo[1,5-c]pyrimidin-7-yl)benzonitrile;
3-(5-amino-8-(3-methylpyridin-4-yl)-2-(pyridin-2-ylamino)-[1,2,4]triazolo[1,5-c]pyrimidin-7-yl)benzonitrile;
3-(5-amino-8-(2,6-dimethylpyridin-4-yl)-2-(pyridin-2-ylamino)-[1,2,4]triazolo[1,5-c]pyrimidin-7-yl)benzonitrile;
3-(5-amino-8-(2-methoxy-6-methylpyridin-4-yl)-2-(pyridin-2-ylamino)-[1,2,4]triazolo[1,5-c]pyrimidin-7-yl)benzonitrile;
3-(5-amino-2-((6-methylpyridin-2-yl)amino)-8-(pyrimidin-4-yl)-[1,2,4]triazolo[1,5-c]pyrimidin-7-yl)benzonitrile;
3-(5-amino-2-((pyridin-2-yloxy)methyl)-8-(pyrimidin-4-yl)-[1,2,4]triazolo[1,5-c]pyrimidin-7-yl)benzonitrile;
2-((5-amino-7-(3-cyanophenyl)-8-(pyrimidin-4-yl)-[1,2,4]triazolo[1,5-c]pyrimidin-2-yl)methoxy)nicotinonitrile;
2-((5-amino-7-(3-cyanophenyl)-8-(1-methyl-6-oxo-1,6-dihydropyridin-3-yl)-[1,2,4]triazolo[1,5-c]pyrimidin-2-yl)methoxy)nicotinonitrile;
2-((5-amino-7-(3-cyanophenyl)-8-(1-methyl-6-oxo-1,6-dihydropyridazin-3-yl)-[1,2,4]triazolo[1,5-c]pyrimidin-2-yl)methoxy)nicotinonitrile;
2-((5-amino-7-(3-cyanophenyl)-8-(3-methylpyridin-4-yl)-[1,2,4]triazolo[1,5-c]pyrimidin-2-yl)methoxy)nicotinonitrile;
2-((5-amino-7-(3-cyanophenyl)-8-(2,6-dimethylpyridin-4-yl)-[1,2,4]triazolo[1,5-c]pyrimidin-2-yl)methoxy)nicotinonitrile;
2-((5-amino-7-(3-cyanophenyl)-8-(2-methoxy-6-methylpyridin-4-yl)-[1,2,4]triazolo[1,5-c]pyrimidin-2-yl)methoxy)nicotinonitrile;
3-(5-amino-2-((1-(pyridin-2-yl)ethyl)amino)-8-(pyrimidin-4-yl)-[1,2,4]triazolo[1,5-c]pyrimidin-7-yl)benzonitrile;
3-(5-amino-2-((2-(pyridin-2-yl)propan-2-yl)amino)-8-(pyrimidin-4-yl)-[1,2,4]triazolo[1,5-c]pyrimidin-7-yl)benzonitrile;
3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile;
3-(5-amino-2-((5-(pyrimidin-2-yl)-1H-tetrazol-1-yl)methyl)-8-(pyrimidin-4-yl)-[1,2,4]triazolo[1,5-c]pyrimidin-7-yl)benzonitrile;
3-(5-amino-8-(pyrimidin-4-yl)-2-((5-(pyrimidin-4-yl)-1H-tetrazol-1-yl)methyl)-[1,2,4]triazolo[1,5-c]pyrimidin-7-yl)benzonitrile;
3-(5-amino-2-((5-(pyridin-3-yl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(pyridin-4-yl)-1H-tetrazol-1-yl)methyl)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-((6-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridazin-3-yl)-2-((6-methylpyridin-2-yl)methoxy)-[1,2,4]triazolo[1,5-c]pyrimidin-7-yl)benzonitrile;
3-(5-amino-2-((6-methylpyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((6-methylpyridin-2-yl)methoxy)-[1,2,4]triazolo[1,5-c]pyrimidin-7-yl)benzonitrile;
3-(5-amino-8-(2-methoxy-6-methylpyridin-4-yl)-2-((6-methylpyridin-2-yl)methoxy)-[1,2,4]triazolo[1,5-c]pyrimidin-7-yl)benzonitrile;
3-(5-amino-2-((3,6-dimethylpyridin-2-yl)methoxy)-8-(1-methyl-6-oxo-1,6-dihydropyridin-3-yl)-[1,2,4]triazolo[1,5-c]pyrimidin-7-yl)benzonitrile;
3-(5-amino-2-((3,6-dimethylpyridin-2-yl)methoxy)-8-(1-methyl-6-oxo-1,6-dihydropyridazin-3-yl)-[1,2,4]triazolo[1,5-c]pyrimidin-7-yl)benzonitrile;
3-(5-amino-2-((3,6-dimethylpyridin-2-yl)methoxy)-8-(3-methylpyridin-4-yl)-[1,2,4]triazolo[1,5-c]pyrimidin-7-yl)benzonitrile;
3-(5-amino-2-((3,6-dimethylpyridin-2-yl)methoxy)-8-(2,6-dimethylpyridin-4-yl)-[1,2,4]triazolo[1,5-c]pyrimidin-7-yl)benzonitrile;
3-(5-amino-2-((3,6-dimethylpyridin-2-yl)methoxy)-8-(2-methoxy-6-methylpyridin-4-yl)-[1,2,4]triazolo[1,5-c]pyrimidin-7-yl)benzonitrile;
3-(5-amino-2-(((6-methylpyridin-2-yl)methyl)amino)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-(((6-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridazin-3-yl)-2-(((6-methylpyridin-2-yl)methyl)amino)-[1,2,4]triazolo[1,5-c]pyrimidin-7-yl)benzonitrile;
3-(5-amino-2-(((6-methylpyridin-2-yl)methyl)amino)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-(((6-methylpyridin-2-yl)methyl)amino)-[1,2,4]triazolo[1,5-c]pyrimidin-7-yl)benzonitrile; and
3-(5-amino-8-(2-methoxy-6-methylpyridin-4-yl)-2-(((6-methylpyridin-2-yl)methyl)amino)-[1,2,4]triazolo[1,5-c]pyrimidin-7-yl)benzonitrile;

or a pharmaceutically acceptable salt thereof.

In some embodiments, the compound of Formula (I) is selected from:
3-(5-amino-2-((5-(3-aminophenyl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(6-methylpyridin-2-yl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(6-methoxypyridin-2-yl)-1H-tetrazol-1-yl)methyl)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyridin-2-yl)-1H-tetrazol-1-yl)methyl)-[1,2,4]triazolo[1,5-c]pyrimidin-7-yl)benzonitrile;
3-(2-((5-(1H-pyrazol-1-yl)-1H-tetrazol-1-yl)methyl)-5-amino-8-(1-methyl-6-oxo-1,6-dihydropyridin-3-yl)-[1,2,4]triazolo[1,5-c]pyrimidin-7-yl)benzonitrile;
3-(5-amino-8-(1-methyl-6-oxo-1,6-dihydropyridin-3-yl)-2-((5-(thiazol-4-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyrimidin-2-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyrazin-2-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridazin-3-yl)-2-((5-(pyridin-2-yl)-1H-tetrazol-1-yl)methyl)-[1,2,4]triazolo[1,5-c]pyrimidin-7-yl)benzonitrile;
3-(5-amino-2-((5-(pyridin-2-yl)-1H-tetrazol-1-yl)methyl)-8-(pyridin-4-yl)-[1,2,4]triazolo[1,5-c]pyrimidin-7-yl)benzonitrile;
3-(5-amino-8-(2-(dimethylamino)pyridin-4-yl)-2-((5-(pyridin-2-yl)-1H-tetrazol-1-yl)methyl)-[1,2,4]triazolo[1,5-c]pyrimidin-7-yl)benzonitrile;
3-(5-amino-8-(2-aminopyridin-4-yl)-2-((5-(pyridin-2-yl)-1H-tetrazol-1-yl)methyl)-[1,2,4]triazolo[1,5-c]pyrimidin-7-yl)benzonitrile;
3-(5-amino-2-(2-fluoro-6-(pyridin-4-yl)benzyl)-8-(pyridin-4-yl)-[1,2,4]triazolo[1,5-c]pyrimidin-7-yl)benzonitrile;
3-(5-amino-8-(2-aminopyridin-4-yl)-2-(2-(2-aminopyridin-4-yl)-6-fluorobenzyl)-[1,2,4]triazolo[1,5-c]pyrimidin-7-yl)benzonitrile;
3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)benzonitrile;
3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridin-3-yl)-2-((3-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridazin-3-yl)-2-((3-methylpyridin-2-yl)methoxy)-[1,2,4]triazolo[1,5-c]pyrimidin-7-yl)benzonitrile;
3-(5-amino-2-((3-methylpyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((3-methylpyridin-2-yl)methoxy)-[1,2,4]triazolo[1,5-c]pyrimidin-7-yl)benzonitrile;
3-(5-amino-8-(1-ethyl-6-oxo-1,6-dihydropyridin-3-yl)-2-((3-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(2-methoxy-6-methylpyridin-4-yl)-2-(((3-methylpyridin-2-yl)methyl)amino)-[1,2,4]triazolo[1,5-c]pyrimidin-7-yl)-2-fluorobenzonitrile;
3-(5-amino-8-(1-methyl-6-oxo-1,6-dihydropyridazin-3-yl)-2-(pyridin-2-ylmethyl)-[1,2,4]triazolo[1,5-c]pyrimidin-7-yl)benzonitrile;
3-(5-amino-2-((3-fluoropyridin-2-yl)methoxy)-8-(pyrimidin-4-yl)-[1,2,4]triazolo[1,5-c]pyrimidin-7-yl)benzonitrile;
3-(5-amino-2-((3-fluoropyridin-2-yl)methoxy)-8-(1-methyl-6-oxo-1,6-dihydropyridin-3-yl)-[1,2,4]triazolo[1,5-c]pyrimidin-7-yl)benzonitrile;
3-(5-amino-2-((3-fluoropyridin-2-yl)methoxy)-8-(1-methyl-6-oxo-1,6-dihydropyridazin-3-yl)-[1,2,4]triazolo[1,5-c]pyrimidin-7-yl)benzonitrile;
3-(5-amino-8-(2,6-dimethylpyridin-4-yl)-2-((3-fluoropyridin-2-yl)methoxy)-[1,2,4]triazolo[1,5-c]pyrimidin-7-yl)benzonitrile;
3-(5-amino-2-((3-fluoropyridin-2-yl)methoxy)-8-(2-methoxy-6-methylpyridin-4-yl)-[1,2,4]triazolo[1,5-c]pyrimidin-7-yl)benzonitrile;
3-(5-amino-2-(pyridin-2-ylamino)-8-(pyrimidin-4-yl)-[1,2,4]triazolo[1,5-c]pyrimidin-7-yl)benzonitrile;
3-(5-amino-8-(2-methoxy-6-methylpyridin-4-yl)-2-(pyridin-2-ylamino)-[1,2,4]triazolo[1,5-c]pyrimidin-7-yl)benzonitrile;
3-(5-amino-2-((6-methylpyridin-2-yl)amino)-8-(pyrimidin-4-yl)-[1,2,4]triazolo[1,5-c]pyrimidin-7-yl)benzonitrile;
3-(5-amino-2-((pyridin-2-yloxy)methyl)-8-(pyrimidin-4-yl)-[1,2,4]triazolo[1,5-c]pyrimidin-7-yl)benzonitrile;
2-((5-amino-7-(3-cyanophenyl)-8-(pyrimidin-4-yl)-[1,2,4]triazolo[1,5-c]pyrimidin-2-yl)methoxy)nicotinonitrile;
2-((5-amino-7-(3-cyanophenyl)-8-(1-methyl-6-oxo-1,6-dihydropyridin-3-yl)-[1,2,4]triazolo[1,5-c]pyrimidin-2-yl)methoxy)nicotinonitrile;
2-((5-amino-7-(3-cyanophenyl)-8-(1-methyl-6-oxo-1,6-dihydropyridazin-3-yl)-[1,2,4]triazolo[1,5-c]pyrimidin-2-yl)methoxy)nicotinonitrile;
2-((5-amino-7-(3-cyanophenyl)-8-(2,6-dimethylpyridin-4-yl)-[1,2,4]triazolo[1,5-c]pyrimidin-2-yl)methoxy)nicotinonitrile;
2-((5-amino-7-(3-cyanophenyl)-8-(2-methoxy-6-methylpyridin-4-yl)-[1,2,4]triazolo[1,5-c]pyrimidin-2-yl)methoxy)nicotinonitrile;
3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile;
3-(5-amino-2-((5-(pyrimidin-2-yl)-1H-tetrazol-1-yl)methyl)-8-(pyrimidin-4-yl)-[1,2,4]triazolo[1,5-c]pyrimidin-7-yl)benzonitrile;
3-(5-amino-8-(pyrimidin-4-yl)-2-((5-(pyrimidin-4-yl)-1H-tetrazol-1-yl)methyl)-[1,2,4]triazolo[1,5-c]pyrimidin-7-yl)benzonitrile;
3-(5-amino-2-((5-(pyridin-3-yl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(pyridin-4-yl)-1H-tetrazol-1-yl)methyl)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-((6-methylpyridin-2-yl)methoxy)-[1,2,4]triazolo[1,5-c]pyrimidin-7-yl)benzonitrile;
3-(5-amino-2-((6-methylpyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((6-methylpyridin-2-yl)methoxy)-[1,2,4]triazolo[1,5-c]pyrimidin-7-yl)benzonitrile;
3-(5-amino-8-(2-methoxy-6-methylpyridin-4-yl)-2-((6-methylpyridin-2-yl)methoxy)-[1,2,4]triazolo[1,5-c]pyrimidin-7-yl)benzonitrile;
3-(5-amino-2-(((6-methylpyridin-2-yl)methyl)amino)-8-(pyrimidin-4-yl)-[1,2,4]triazolo[1,5-c]pyrimidin-7-yl)benzonitrile;
3-(5-amino-8-(1-methyl-6-oxo-1,6-dihydropyridazin-3-yl)-2-(((6-methylpyridin-2-yl)methyl)amino)-[1,2,4]triazolo[1,5-c]pyrimidin-7-yl)benzonitrile;
3-(5-amino-2-(((6-methylpyridin-2-yl)methyl)amino)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-(((6-methylpyridin-2-yl)methyl)amino)-[1,2,4]triazolo[1,5-c]pyrimidin-7-yl)benzonitrile; and
3-(5-amino-8-(2-methoxy-6-methylpyridin-4-yl)-2-(((6-methylpyridin-2-yl)methyl)amino)-[1,2,4]triazolo[1,5-c]pyrimidin-7-yl)benzonitrile;

or a pharmaceutically acceptable salt thereof.

In some embodiments, the compound is the (S)-enantiomer of one of the preceding compounds, or a pharmaceutically acceptable salt thereof. In some embodiments, the compound is the (R)-enantiomer of one of the preceding compounds, or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.” Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, “halo” or “halogen” refers to F, Cl, Br, or I. In some embodiments, a halo is F, Cl, or Br. In some embodiments, a halo is F or Cl. In some embodiments, a halo is F. In some embodiments, a halo is Cl.

As used herein, “heteroaryl” refers to a monocyclic aromatic heterocycle having at least one heteroatom ring member selected from N, O, S, and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a five-membered heteroaryl ring. In some embodiments, the heteroaryl is a six-membered heteroaryl ring. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridine, pyrimidine, pyrazine, pyridazine, dihydropyridine, dihydropyridazine, pyrrole, pyrazole, azolyl, oxazole, isoxazole, thiazole, isothiazole, imidazole, furan, thiophene, triazole, tetrazole, thiadiazole, triazine.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as 0-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

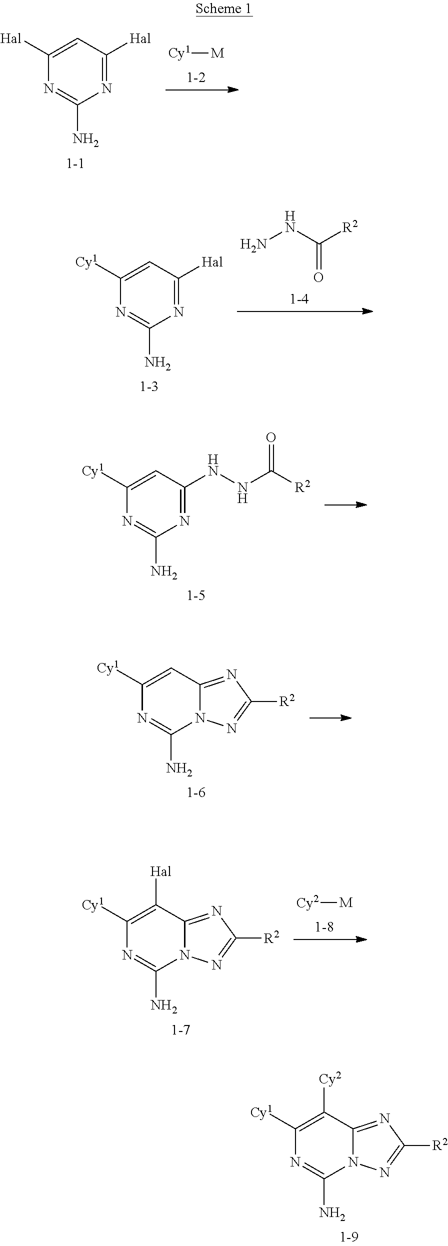

Compounds of formula 1-9 can be synthesized via the synthetic route outlined in Scheme 1. Starting material 1-1 first undergoes a cross-coupling reaction with reagent 1-2 to generate compound 1-3, in which M is a boronic acid, boronic ester or an appropriately substituted metal [e.g., M is B(OR)₂, Sn(Alkyl)₃, or Zn-Hal], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst). A nucleophilic aromatic substitution (S_{N}Ar) reaction of compound 1-3 with hydrazide 1-4 then affords compound 1-5, which undergoes a cyclization reaction at elevated temperature in the presence of a suitable reagent, such as N,O-bis(trimethylsilyl)acetamide, to produce bicycle 1-6. Halogenation of 1-6 with an appropriate reagent, such as N-bromosuccinimide (NBS), affords compound 1-7. The final product 1-9 can be prepared by a cross-coupling reaction between compound 1-7 and a derivative of formula 1-8, using similar procedures as described for the preparation of compound 1-3 from starting material 1-1. At various stages during this synthetic sequence, the R² group can be further functionalized.

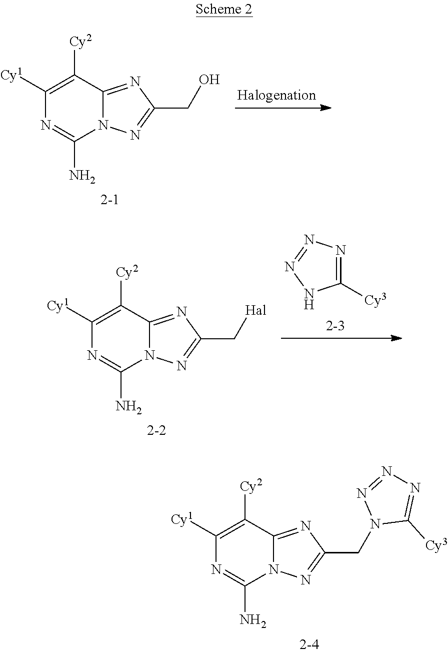

Compounds of formula 2-4 can be synthesized via the synthetic route outlined in Scheme 2. Advanced intermediate 2-1 (which can be prepared using synthetic procedures as outlined in Scheme 1) first undergoes a halogenation reaction (using an suitable reagent, such as thionyl chloride) to generate compound 2-2 (Hal is a halide, such as F, Cl, Br, or I). Compound 2-2 can then be subjected to a nucleophilic substitution reaction (S_{N}2) with reagents of formula 2-3, to afford compound 2-4.

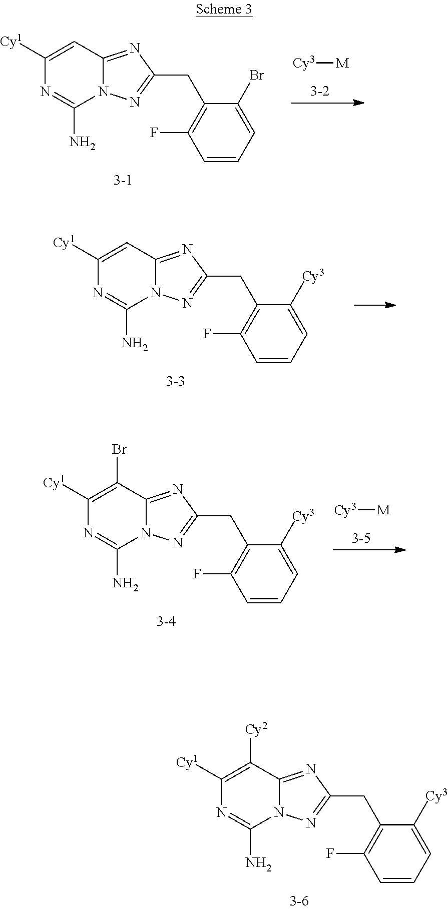

Compounds of formula 3-6 can be synthesized via the synthetic route outlined in Scheme 3. Advanced intermediate 3-1 (which can be prepared using synthetic procedures as outlined in Scheme 1) first undergoes a cross-coupling reaction with reagent 3-2 to generate compound 3-3, in which M is a boronic acid, boronic ester or an appropriately substituted metal [e.g., M is B(OR)₂, Sn(Alkyl)₃, or Zn-Hal], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst). Bromination reaction (NBS) of 3-3 generates compound 3-4. Compound 3-4 can then be subjected to a cross-coupling reaction with reagent 3-5, using similar procedures as described for the preparation of compound 3-3 from 3-1, to afford compound 3-6.

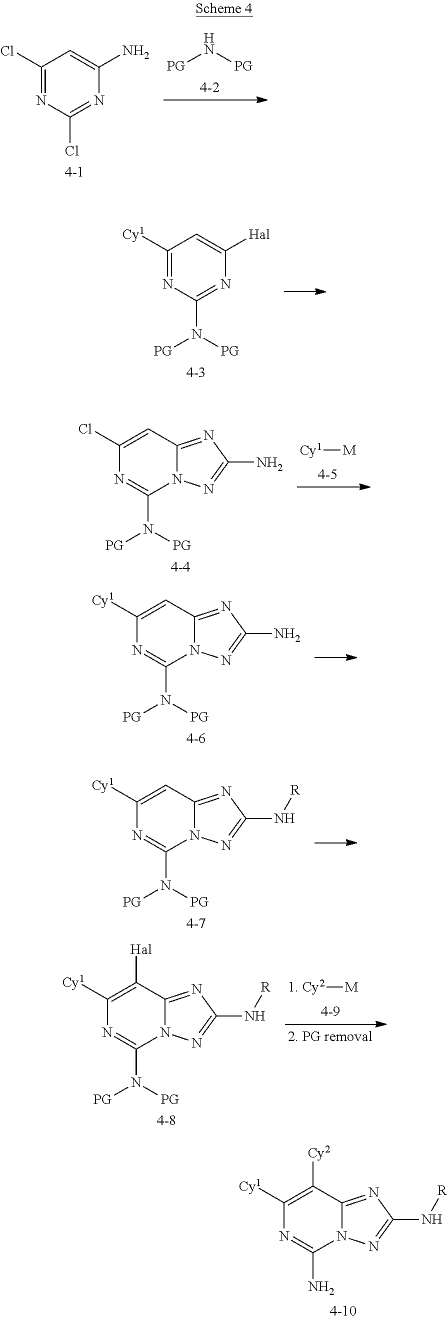

Compounds of formula 4-10 can be synthesized via the synthetic route outlined in Scheme 4. Selective nucleophilic aromatic substitution (S_{N}Ar) reaction of starting material 4-1 with amine 4-2 (PG represents a suitable protecting group, such as 4-methoxybenzyl) affords compound 4-3. Compound 4-3 can then be cyclized to intermediate 4-4 via appropriate chemical transformations, such as a two-step sequence using O-ethyl carbonisothiocyanatidate and hydroxylamine hydrochoride. A cross-coupling reaction between 4-4 and a reagent of formula 4-5, in which M is a boronic acid, boronic ester or an appropriately substituted metal [e.g., M is B(OR)₂, Sn(Alkyl)₃, or Zn-Hal], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), will generate intermediate 4-6. The amino group of 4-6 can then be functionalized using suitable chemical transformations, such as Buchwald-Hartwig coupling conditions in the presence of a palladium catalyst (e.g., chloro(2-dicyclohexylphosphino-2′,4′,6′-triisopropyl-1,1′-biphenyl)[2-(2′-amino-1,1′-biphenyl)]palladium(II)) and a base (e.g., sodium tert-butoxide), or reductive amination conditions (e.g., in the presence of a suitable hydride source) to afford 4-7. Halogenation of 4-7 using a suitable reagent, such as N-bromosuccinimide (NBS), gives compound 4-8. A cross-coupling reaction between 4-8 and a derivative of formula 4-9, using similar procedures as described for the preparation of compound 4-6 from compound 4-4, followed by protecting group removal affords product 4-10.

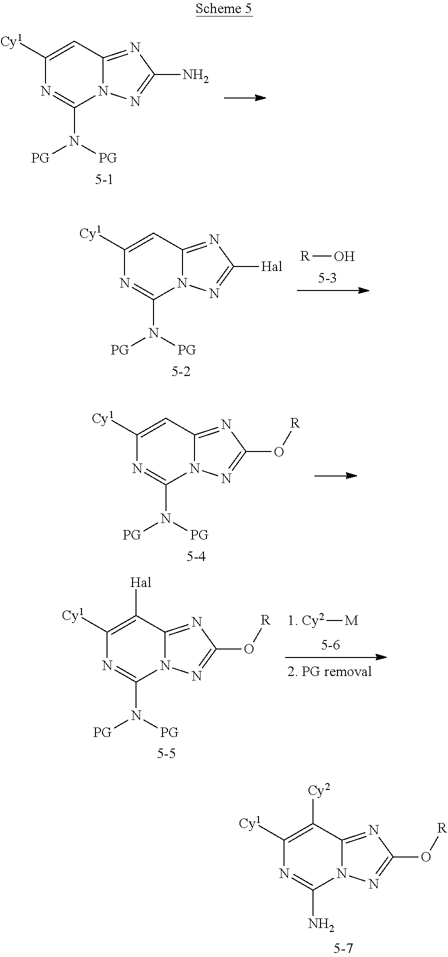

Compounds of formula 5-7 can be synthesized via the synthetic route outlined in Scheme 5. The amino group of 5-1 (which can be prepared using synthetic procedures as outlined in Scheme 4) can first be converted to halogen using suitable chemical transformations, such as Sandmeyer Reaction (e.g., in the presence of a suitable oxidant such as isobutyl nitrite and a suitable halogen source) to afford 5-2. A nucleophilic aromatic substitution (S_{N}Ar) reaction of compound 5-2 with alcohol 5-3 in the presence of a suitable base then affords compound 5-4. Halogenation of 5-4 using a suitable reagent, such as N-bromosuccinimide (NBS), gives compound 5-5. A cross-coupling reaction between 5-5 and a derivative of formula 5-6, followed by protecting group removal, affords product 5-7.

### Methods of Use

The compounds of the present disclosure can modulate the activity of adenosine receptors, such as subtypes A2A and A2B receptors. Accordingly, the compounds, salts or stereoisomers described herein can be used in methods of inhibiting adenosine receptors (e.g., A2A and/or A2B receptors) by contacting the receptor with any one or more of the compounds, salts, or compositions described herein. In some embodiments, the compounds or salts can be used in methods of inhibiting activity of an adenosine receptor in an individual/patient in need of the inhibition by administering an effective amount of a compound or salt of described herein. In some embodiments, modulating is inhibiting. In some embodiments, the contacting is in vivo. In some embodiments, the contacting is ex vivo or in vitro.

The compounds or salts described herein can be selective. By “selective,” it is meant that the compound binds to or inhibits an adenosine receptor with greater affinity or potency, respectively, compared to at least one other receptor, kinase, etc. The compounds of the present disclosure can also be dual antagonists (i.e., inhibitors) of adenosine receptors, e.g., A2A and A2B adenosine receptors.

Another aspect of the present disclosure pertains to methods of treating an adenosine receptor associated disease or disorder in an individual (e.g., patient) by administering to the individual in need of such treatment a therapeutically effective amount or dose of one or more compounds of the present disclosure or a pharmaceutical composition thereof. An adenosine receptor associated disease or disorder can include any disease, disorder or condition that is directly or indirectly linked to expression or activity of the adenosine receptor, including overexpression and/or abnormal activity levels.

The compounds of the present disclosure are useful in the treatment of diseases related to the activity of adenosine receptors including, for example, cancer, inflammatory diseases, cardiovascular diseases, neurodegenerative diseases, immunomodulatory disorders, central nerve system diseases, and diabetes.

Based on the compelling roles of adenosine, e.g., A2A, A2B, receptors in multiple immunosuppressive mechanisms, developing inhibitors can boost the immune system to suppress tumor progression. Adenosine receptor inhibitors can be used to treat, alone or in combination with other therapies, bladder cancer, lung cancer (e.g., non-small cell lung cancer (NSCLC), lung metastasis), melanoma (e.g., metastatic melanoma), breast cancer, cervical cancer, ovarian cancer, colorectal cancer, pancreatic cancer, esophageal cancer, prostate cancer, kidney cancer, skin cancer, thyroid cancer, liver cancer, uterine cancer, head and neck cancer, and renal cell carcinoma (Antonioli, L. et al., Nature Reviews Cancer, 2013, 13, 842-857). See also, https://globenewswire.com/news-release/2017/04/04/954192/0/en/Corvus-Pharmaceuticals-Announces-Interim-Results-from-Ongoing-Phase-1-1b-Study-Demonstrating-Safety-and-Clinical-Activity-of-Lead-Checkpoint-Inhibitor-CPI-444-in-Patients-with-Adva.html; Cekic C. et al., J Immunol, 2012, 188:198-205; Iannone, R. et al., Am. J. Cancer Res. 2014, 4:172-181 (study shows that both A2A and CD73 blockade enhance the antitumor activity of anti-CTLA-4 mAb therapy in a B16F10 murine melanoma model); Iannone, R. et al., Neoplasia, 2013, 15:1400-1410 and Beavis P A., et al., Proc Natl Acad Sci. USA, 2013, 110:14711-14716 (study shows that A2A and CD73 blockade decreased metastasis in 4T1 breast tumor model with has high CD73 expression). In some embodiments, the prostate cancer is metastatic castrate-resistant prostate carcinoma (mCRPC). In some embodiments, the colorectal cancer is colorectal carcinoma (CRC).

In some embodiments, the disease or disorder is lung cancer (e.g., non-small cell lung cancer), melanoma, pancreatic cancer, breast cancer, head and neck squamous cell carcinoma, prostate cancer, liver cancer, color cancer, endometrial cancer, bladder cancer, skin cancer, cancer of the uterus, renal cancer, gastric cancer, or sarcoma. In some embodiments, the sarcoma is Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, or undifferentiated pleomorphic sarcoma.

In some embodiments, the disease or disorder is mesothelioma or adrenocarcinoma. In some embodiments, the disease or disorder is mesothelioma. In some embodiments, the disease or disorder is adrenocarcinoma.

MDSC (myeloid-derived suppressor cells) are a heterogenous group of immune cells from the myeloid lineage (a family of cells that originate from bone marrow stem cells). MDSCs strongly expand in pathological situations such as chronic infections and cancer, as a result of an altered haematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions. In some embodiments, the compounds, etc. described herein can be used in methods related to cancer tissue (e.g., tumors) with high infiltration of MDSCs, including Solid tumors with high basal level of macrophage and/or MDSC infiltration.

In some embodiments, the disease or disorder is head and neck squamous cell carcinoma (HNSCC), non-small cell lung cancer (NSCLC), colorectal cancer, melanoma, ovarian cancer, bladder cancer, renal cell carcinoma, liver cancer, or hepatocellular carcinoma.

In some embodiments, the compounds of the disclosure can be used in treating pulmonary inflammation, including bleomycin-induced pulmonary fibrosis and injury related to adenosine deaminase deficiency (Baraldi, et al., Chem. Rev., 2008, 108, 238-263).

In some embodiments, the compounds of the disclosure can be used as a treatment for inflammatory disease such as allergic reactions (e.g., A2B adenosine receptor dependent allergic reactions) and other adenosine receptor dependent immune reactions. Further inflammatory diseases that can be treated by compounds of the disclosure include respiratory disorders, sepsis, reperfusion injury, and thrombosis.

In some embodiments, the compounds of the disclosure can be used as a treatment for cardiovascular disease such as coronary artery disease (myocardial infarction, angina pectoris, heart failure), cerebrovascular disease (stroke, transient ischemic attack), peripheral artery disease, and aortic atherosclerosis and aneurysm. Atherosclerosis is an underlying etiologic factor in many types of cardiovascular disease. Atherosclerosis begins in adolescence with fatty streaks, which progress to plaques in adulthood and finally results in thrombotic events that cause occlusion of vessels leading to clinically significant morbidity and mortality. Antagonists to the A2B adenosine receptor and A2A adenosine receptor may be beneficial in preventing atherosclerotic plaque formation (Eisenstein, A. et al., J. Cell Physiol., 2015, 230(12), 2891-2897).

In some embodiments, the compounds of the disclosure can be used as a treatment for disorders in motor activity; deficiency caused by degeneration of the striatonigral dopamine system; and Parkinson's disease; some of the motivational symptoms of depression (Collins, L. E. et al. Pharmacol. Biochem. Behav., 2012, 100, 498-505.).

In some embodiments, the compounds of the disclosure can be used as a treatment for diabetes and related disorders, such as insulin resistance. Diabetes affects the production of adenosine and the expression of A2B adenosine receptors (A2BRs) that stimulate IL-6 and CRP production, insulin resistance, and the association between A_{2B}R gene single-nucleotide polymorphisms (ADORA2B SNPs) and inflammatory markers.

The increased A2BR signaling in diabetes may increase insulin resistance in part by elevating pro-inflammatory mediators. Selective A2BR blockers may be useful to treat insulin resistance (Figler, R. A. et al. Diabetes, 2011, 60 (2), 669-679).

It is believed that compounds provided herein, e.g., compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual” or “patient”, used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

### I. Immune-Checkpoint Therapies

In some embodiments, A2A and A2B dual inhibitors provided herein can be used in combination with one or more immune checkpoint inhibitors for the treatment of cancer as described herein. In one embodiment, the combination with one or more immune checkpoint inhibitors as described herein can be used for the treatment of melanoma. Compounds of the present disclosure can be used in combination with one or more immune checkpoint inhibitors. Exemplary immune checkpoint inhibitors include inhibitors against immune checkpoint molecules such as CD20, CD28, CD40, CD122, CD96, CD73, CD47, GITR, CSF1R, JAK, PI3K delta, PI3K gamma, TAM, arginase, HPK1, CD137 (also known as 4-1B), ICOS, B7-H3, B7-H4, BTLA, CTLA-4, LAG3, TIM3, VISTA, TIGIT, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds of the disclosure provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the A2A and A2B dual inhibitors provided herein can be used in combination with one or more agonists of immune checkpoint molecules, e.g., OX40, CD27, OX40, GITR, and CD137 (also known as 4-1B).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), durvalumab (Imfinzi®), pidilizumab, SHR-1210, PDR001, MGA012, PDR001, AB122, or AMP-224. In some embodiments, the anti-PD-1 monoclonal antibody is nivolumab or pembrolizumab. In some embodiments, the anti-PD1 antibody is pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g. urelumab or utomilumab).

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of PD-1 and PD-L1, e.g., an anti-PD-1/PD-L1 monoclonal antibody. In some embodiments, the anti-PD-1/PD-L1 is MCLA-136.

In some embodiments, the inhibitor is INCB086550.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3, tumor specific antigens (e.g., CD70) or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### II. Cancer Therapies

Cancer cell growth and survival can be impacted by multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The compounds of the present disclosure can be used in combination with one or more other enzyme/protein/receptor inhibitors or one or more therapies for the treatment of diseases, such as cancer. Examples of diseases and indications treatable with combination therapies include those as described herein.

The compounds of the present disclosure can be used in combination with one or more additional pharmaceutical agents such as, for example, chemotherapeutics, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF and FAK kinase inhibitors.

The one or more additional pharmaceutical agents can be administered to a patient simultaneously or sequentially.

For example, the compounds as disclosed herein can be combined with one or more inhibitors of the following kinases for the treatment of cancer and other diseases or disorders described herein: Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFaR, PDGFOR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Non-limiting examples of inhibitors that can be combined with the compounds of the present disclosure for treatment of cancer and other diseases and disorders described herein include an FGFR inhibitor (FGFR1, FGFR2, FGFR3 or FGFR4, e.g., INCB54828, INCB62079 and INCB63904), a JAK inhibitor (JAK1 and/or JAK2, e.g., ruxolitinib, baricitinib or INCB39110), an IDO inhibitor (e.g., epacadostat, NLG919, or BMS-986205), an LSD1 inhibitor (e.g., INCB59872 and INCB60003), a TDO inhibitor, a PI3K-delta inhibitor (e.g., INCB50797 and INCB50465), a Pim inhibitor, a CSF1R inhibitor, a TAM receptor tyrosine kinases (Tyro-3, Axl, and Mer), a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643) and an adenosine receptor antagonist or combinations thereof.

Example antibodies for use in combination therapy include but are not limited to Trastuzumab (e.g. anti-HER2), Ranibizumab (e.g. anti-VEGF-A), Bevacizumab (trade name Avastin, e.g. anti-VEGF, Panitumumab (e.g. anti-EGFR), Cetuximab (e.g. anti-EGFR), Rituxan (anti-CD20) and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Smll, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumortargeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, Toll receptor agonists, STING agonists, oncolytic virotherapy and immunomodulating small molecules, including thalidomide or JAK1/2 inhibitor and the like. The compounds can be administered in combination with one or more anti-cancer drugs, such as a chemotherapeutics. Example chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, olaparib, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, rucaparib, streptozocin, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, niraparib, veliparib, talazoparib, and zoledronate.

Additional examples of chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a PI3K inhibitor of the present disclosure with an additional agent.

In some embodiments, the compounds of the disclosure can be used in combination with an inhibitor of JAK or PI3K6.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, Chlamydia, rickettsial bacteria, mycobacteria, staphylococci, streptococci, pneumonococci, meningococci and conococci, Klebsiella, Proteus, Serratia, Pseudomonas, Legionella, diphtheria, Salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum. Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating A2A and/or A2B receptors in tissue samples, including human, and for identifying A2A and/or A2B antagonists by inhibition binding of a labeled compound. Substitution of one or more of the atoms of the compounds of the present disclosure can also be useful in generating differentiated ADME (Adsorption, Distribution, Metabolism and Excretion.) Accordingly, the present disclosure includes adenosine receptor (e.g., A2A and/or A2B) assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in any of the disclosed Formulas, e.g., Formula (I), can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of any “alkyl”, “alkenyl”, “alkynyl”, “aryl”, “phenyl”, “cycloalkyl”, “heterocycloalkyl”, or “heteroaryl” substituents or “—C₁₋₆ alkyl-”, “alkylene”, “alkenylene” and “alkynylene” linking groups, as described herein, are each optionally replaced by a deuterium atom.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind an adenosine receptor by monitoring its concentration variation when contacting with the adenosine receptor, through tracking of the labeling. For example, a test compound (labeled) can be evaluated for its ability to reduce binding of another compound which is known to bind to an adenosine receptor (i.e., standard compound). Accordingly, the ability of a test compound to compete with the standard compound for binding to the adenosine receptor directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of adenosine receptor-associated diseases or disorders (such as, e.g., cancer, an inflammatory disease, a cardiovascular disease, or a neurodegenerative disease) which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the activity of an adenosine receptor (e.g., A2A and/or A2B) according to at least one assay described herein.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ Cis 5 m, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters XBridge™ C₁₈ 5 m, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)).

Separation of some of the racemic compounds into enantiopure samples were prepared on preparative scale by chiral-phase high performance liquid chromatography under the following conditions: Instrument: Agilent 1100 Prep HPLC; Column: Phenomenex Lux Cellulose-4, 21.2×250 mm, 5 μm; eluting with isocratic mobile phase 45% EtOH in hexanes with a flow rate of 20 mL/minute.

### Example 1. 3-(5-amino-2-((5-(3-aminophenyl)-1H-tetrazol-1-yl)methyl)-8-(pyrimidin-4-yl)-[1,2,4]triazolo[1,5-c]pyrimidin-7-yl)benzonitrile

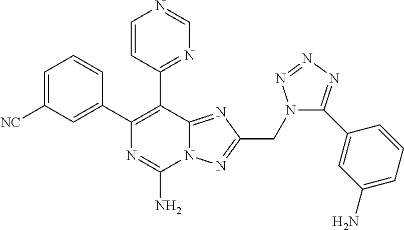 [SMILES: N#Cc1cccc(-c2nc(N)n3nc(Cn4nnnc4-c4cccc(N)c4)nc3c2-c2ccncn2)c1]

### Step 1: 3-(2-Amino-6-chloropyrimidin-4-yl)benzonitrile

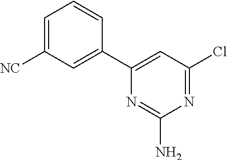 [SMILES: N#Cc1cccc(-c2cc(Cl)nc(N)n2)c1]

A mixture of 4,6-dichloropyrimidin-2-amine (2.5 g, 15.2 mmol), (3-cyanophenyl)boronic acid (2.02 g, 13.7 mmol), tetrakis(triphenylphosphine)palladium(0) (1.06 g, 0.92 mmol) and sodium carbonate (3.23 g, 30.5 mmol) in 1,4-dioxane (60 mL), and water (5 mL) was degassed with nitrogen, then the resulting mixture was heated and stirred at 60° C. for two days. After cooling to room temperature (r.t.), the mixture was concentrated, diluted with water, and extracted with DCM (30 mL×3). The combined organic layers were dried over MgSO₄, filtered, and concentrated. The resulting residue was purified by flash chromatography on a silica gel column eluting with 8% EtOAc in dichloromethane to afford the desired product. LCMS calculated for C₁₁H₈ClN₄ (M+H)⁺: 231.0. Found: 231.0.

### Step 2: 3-(5-Amino-2-(hydroxymethyl)-[1,2,4]triazolo[1,5-c]pyrimidin-7-yl)benzonitrile

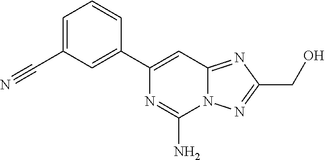 [SMILES: N#Cc1cccc(-c2cc3nc(CO)nn3c(N)n2)c1]

2-Hydroxyacetohydrazide (2.34 g, 26.01 mmol) was added to a ethanol (35 mL) solution of 3-(2-amino-6-chloropyrimidin-4-yl)benzonitrile (4.00 g, 17.34 mmol) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₃H₁₁N₆O (M+H)⁺: 267.1; found 267.1.

### Step 3: 3-(5-Amino-8-bromo-2-(hydroxymethyl)-[1,2,4]triazolo[1,5-c]pyrimidin-7-yl)benzonitrile

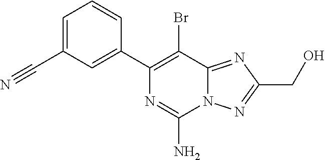 [SMILES: N#Cc1cccc(-c2nc(N)n3nc(CO)nc3c2Br)c1]

To a mixture of 3-(5-amino-2-(hydroxymethyl)-[1,2,4]triazolo[1,5-c]pyrimidin-7-yl)benzonitrile (1.0 g, 3.76 mmol) in DMF (12 mL) at −30° C. was added NBS (0.67 g, 3.76 mmol) portion-wise. The reaction mixture was allowed to slowly warm to 0° C., resulting a homogenous solution. After stirring at 0° C. for 1 h, the reaction mixture was diluted with saturated NaHCO₃ solution and the desired product was collected by filtration and dried. LCMS calculated for C₁₃H₁₀BrN₆O (M+H)⁺: 345.0; found 345.0.

### Step 4: 3-(5-Amino-2-(hydroxymethyl)-8-(pyrimidin-4-yl)-[1,2,4]triazolo[1,5-c]pyrimidin-7-yl)benzonitrile

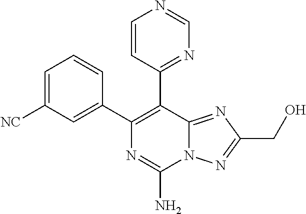 [SMILES: N#Cc1cccc(-c2nc(N)n3nc(CO)nc3c2-c2ccncn2)c1]

Tetrakis(triphenylphosphine)palladium(0) (0.067 g, 0.058 mmol) was added to a mixture of 4-(tributylstannyl)pyrimidine (0.321 g, 0.869 mmol), 3-(5-amino-8-bromo-2-(hydroxymethyl)-[1,2,4]triazolo[1,5-c]pyrimidin-7-yl)benzonitrile (0.20 g, 0.579 mmol), CsF (0.176 g, 1.159 mmol), and copper(I)iodide (0.022 g, 0.116 mmol) in 1,4-dioxane (5.0 mL). The reaction mixture was purged with N₂ and stirred at 80° C. for 7 h. The resulting mixture was cooled to r.t., concentrated and purified by flash column chromatography eluting with 0% to 10% methanol in DCM to afford the product. LC-MS calculated for C₁₇H₁₃N₈O (M+H)⁺: 345.1; found 345.1.

### Step 5: 3-(5-Amino-2-(chloromethyl)-8-(pyrimidin-4-yl)-[1,2,4]triazolo[1,5-c]pyrimidin-7-yl)benzonitrile

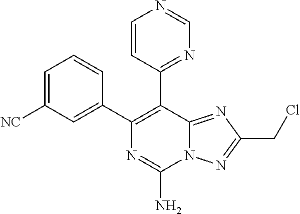 [SMILES: N#Cc1cccc(-c2nc(N)n3nc(CCl)nc3c2-c2ccncn2)c1]

To a mixture of 3-(5-amino-2-(hydroxymethyl)-8-(pyrimidin-4-yl)-[1,2,4]triazolo[1,5-c]pyrimidin-7-yl)benzonitrile (0.1 g, 0.290 mmol) in Acetonitrile (10 ml) was added thionyl chloride (0.212 ml, 2.90 mmol) at r.t. The reaction mixture was stirred at r.t. for 5 h, concentrated, and purified by flash chromatography eluting with 0% to 5% methanol in DCM to afford the product. LC-MS calculated for C₁₇H₁₂ClN₈ (M+H)⁺: 363.1; found 363.1.

### Step 6: 3-(5-amino-2-((5-(3-aminophenyl)-H-tetrazol-1-yl)methyl)-8-(pyrimidin-4-yl)-[1,2,4]triazolo[1,5-c]pyrimidin-7-yl)benzonitrile

A mixture of 3-(5-amino-2-(chloromethyl)-8-(pyrimidin-4-yl)-[1,2,4]triazolo[1,5-c]pyrimidin-7-yl)benzonitrile (10 mg, 0.028 mmol), 3-(1H-tetrazol-5-yl)aniline (8.9 mg, 0.055 mmol) and Cs₂CO₃ (20.7 mg, 0.064 mmol) in DMF (1 mL) was stirred at 100° C. for 10 min. The reaction mixture was then cooled to r.t., diluted with methanol (4 mL), and purified by preparative LC-MS (pH 2, acetonitrile/water with TFA) to afford the product as a TFA salt. LCMS calculated for C₂₄H₁₈N₁₃ (M+H)⁺: 488.2; found 488.2.

### Example 2. 3-(5-amino-2-((5-(6-methylpyridin-2-yl)-1H-tetrazol-1-yl)methyl)-8-(pyrimidin-4-yl)-[1,2,4]triazolo[1,5-c]pyrimidin-7-yl)benzonitrile

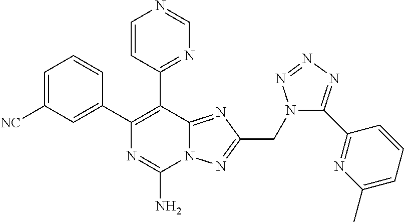 [SMILES: Cc1cccc(-c2nnnn2Cc2nc3c(-c4ccncn4)c(-c4cccc(C#N)c4)nc(N)n3n2)n1]

This compound was prepared using similar procedures as described for Example 1, with 5-(m-tolyl)-1H-tetrazole replacing 3-(1H-tetrazol-5-yl)aniline. The product was purified by preparative LC-MS (pH 2, acetonitrile/water with TFA) to afford the product as a TFA salt. LCMS calculated for C₂₄H₁₈N₁₃ (M+H)⁺: 488.2; found 488.2.

### Example 3. 3-(5-amino-2-((5-(6-methoxypyridin-2-yl)-1H-tetrazol-1-yl)methyl)-8-(pyrimidin-4-yl)-[1,2,4]triazolo[1,5-c]pyrimidin-7-yl)benzonitrile

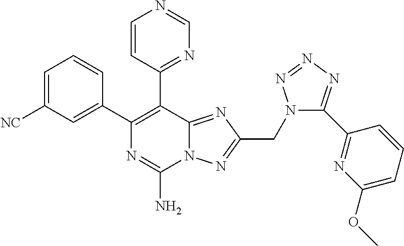 [SMILES: COc1cccc(-c2nnnn2Cc2nc3c(-c4ccncn4)c(-c4cccc(C#N)c4)nc(N)n3n2)n1]

This compound was prepared using similar procedures as described for Example 1, with 2-methoxy-6-(1H-tetrazol-5-yl)pyridine replacing 3-(1H-tetrazol-5-yl)aniline. The product was purified by preparative LC-MS (pH 2, acetonitrile/water with TFA) to afford the product as a TFA salt. LCMS calculated for C₂₄H₁₈N₁₃O (M+H)⁺: 504.2; found 504.2

### Example 4. 3-(5-amino-8-(1-methyl-6-oxo-1,6-dihydropyridin-3-yl)-2-((5-(pyridin-2-yl)-1H-tetrazol-1-yl)methyl)-[1,2,4]triazolo[1,5-c]pyrimidin-7-yl)benzonitrile

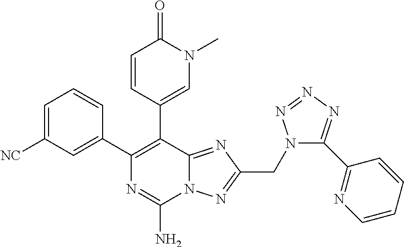 [SMILES: Cn1cc(-c2c(-c3cccc(C#N)c3)nc(N)n3nc(Cn4nnnc4-c4ccccn4)nc23)ccc1=O]

### Step 1: 3-(5-amino-2-(hydroxymethyl)-8-(1-methyl-6-oxo-1,6-dihydropyridin-3-yl)-[1,2,4]triazolo[1,5-c]pyrimidin-7-yl)benzonitrile

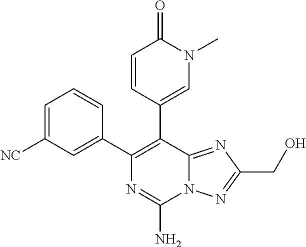 [SMILES: Cn1cc(-c2c(-c3cccc(C#N)c3)nc(N)n3nc(CO)nc23)ccc1=O]

A mixture of 3-(5-amino-8-bromo-2-(hydroxymethyl)-[1,2,4]triazolo[1,5-c]pyrimidin-7-yl)benzonitrile (20 mg, 0.046 mmol), 1-methyl-5-(4,4,5,5-tetramethyl-1,3,2-dioxaborolan-2-yl)pyridin-2(1H)-one (100 mg, 0.46 mmol), XPhos Pd G2 (35 mg, 47 μmol), and Na₂CO₃ (200 mg, 1.9 mmol) in 1,4-dioxane (5.0 mL) and water (1.0 mL) was flushed with nitrogen and sealed. The reaction mixture was stirred at 110° C. for 1 h. The resulting mixture was cooled to r.t., concentrated and purified by flash column chromatography eluting with 0% to 10% methanol in DCM to afford the product. LC-MS calculated for C₁₉H₁₆N₇O₂ (M+H)⁺: m/z=374.1; found 374.1.

### Step 2: 3-(5-amino-2-(chloromethyl)-8-(I-methyl-6-oxo-1,6-dihydropyridin-3-yl)-[1,2,4]triazolo[1,5-c]pyrimidin-7-yl)benzonitrile

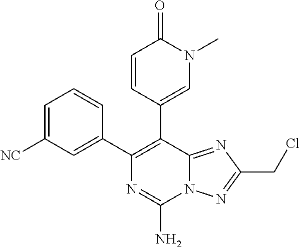 [SMILES: Cn1cc(-c2c(-c3cccc(C#N)c3)nc(N)n3nc(CCl)nc23)ccc1=O]

This compound was prepared using similar procedures as described for Example 1 step 5 with 3-(5-amino-2-(hydroxymethyl)-8-(1-methyl-6-oxo-1,6-dihydropyridin-3-yl)-[1,2,4]triazolo[1,5-c]pyrimidin-7-yl)benzonitrile replacing 3-(5-amino-2-(hydroxymethyl)-8-(pyrimidin-4-yl)-[1,2,4]triazolo[1,5-c]pyrimidin-7-yl)benzonitrile. LCMS calculated for C₁₉H₁₅ClN₇O (M+H)⁺: 392.1; found 392.

### Step 3: 3-(5-amino-2-(chloromethyl)-8-(1-methyl-6-oxo-1,6-dihydropyridin-3-yl)-[1,2,4]triazolo[1,5-c]pyrimidin-7-yl)benzonitrile

This compound was prepared using similar procedures as described for Example 1, step 6 with 3-(5-amino-2-(chloromethyl)-8-(1-methyl-6-oxo-1,6-dihydropyridin-3-yl)-[1,2,4]triazolo[1,5-c]pyrimidin-7-yl)benzonitrile replacing 3-(5-Amino-2-(chloromethyl)-8-(pyrimidin-4-yl)-[1,2,4]triazolo[1,5-c]pyrimidin-7-yl)benzonitrile. The product was purified by preparative LC-MS (pH 2, acetonitrile/water with TFA) to afford the product as a TFA salt. LCMS calculated for C₂₄H₁₈N₁₃O (M+H)⁺: 504.2; found 504.2

### Example 5. 3-(2-((5-(1H-pyrazol-1-yl)-1H-tetrazol-1-yl)methyl)-5-amino-8-(1-methyl-6-oxo-1,6-dihydropyridin-3-yl)-[1,2,4]triazolo[1,5-c]pyrimidin-7-yl)benzonitrile

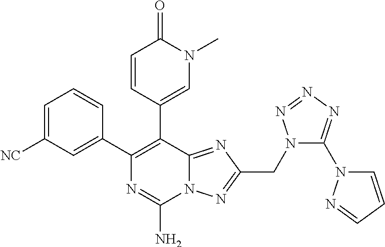 [SMILES: Cn1cc(-c2c(-c3cccc(C#N)c3)nc(N)n3nc(Cn4nnnc4-n4cccn4)nc23)ccc1=O]

This compound was prepared using similar procedures as described for Example 4, with 5-(1H-pyrazol-1-yl)-1H-tetrazole replacing 2-(1H-tetrazol-5-yl)pyridine. The product was purified by preparative LC-MS (pH 2, acetonitrile/water with TFA) to afford the product as a TFA salt. LCMS calculated for C₂₃H₁₈N₁₃O (M+H)⁺: 492.5; found 492.4

### Example 6. 3-(5-amino-8-(1-methyl-6-oxo-1,6-dihydropyridin-3-yl)-2-((5-(thiazol-4-yl)-1H-tetrazol-1-yl)methyl)-[1,2,4]triazolo[1,5-c]pyrimidin-7-yl)benzonitrile

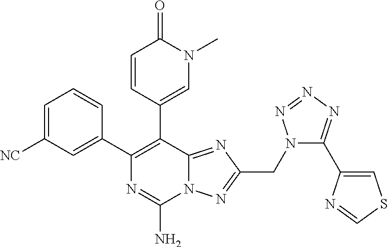 [SMILES: Cn1cc(-c2c(-c3cccc(C#N)c3)nc(N)n3nc(Cn4nnnc4-c4cscn4)nc23)ccc1=O]

This compound was prepared using similar procedures as described for Example 4, with 4-(1H-tetrazol-5-yl)thiazole replacing 2-(1H-tetrazol-5-yl)pyridine. The product was purified by preparative LC-MS (pH 2, acetonitrile/water with TFA) to afford the product as a TFA salt. LCMS calculated for C₂₃H₁₇N₁₂OS (M+H)⁺: 509.1; found 509.2

### Example 7. 3-(5-amino-8-(1-methyl-6-oxo-1,6-dihydropyridin-3-yl)-2-((5-(pyrimidin-2-yl)-1H-tetrazol-1-yl)methyl)-[1,2,4]triazolo[1,5-c]pyrimidin-7-yl)benzonitrile

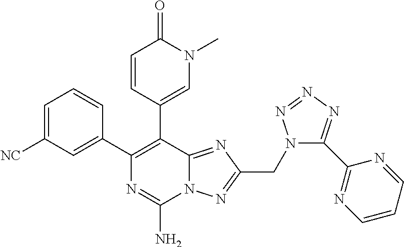 [SMILES: Cn1cc(-c2c(-c3cccc(C#N)c3)nc(N)n3nc(Cn4nnnc4-c4ncccn4)nc23)ccc1=O]

This compound was prepared using similar procedures as described for Example 4, 2-(1H-tetrazol-5-yl)pyrimidine replacing 2-(1H-tetrazol-5-yl)pyridine. The product was purified by preparative LC-MS (pH 2, acetonitrile/water with TFA) to afford the product as a TFA salt. LCMS calculated for C₂₄H₁₈N₁₃O (M+H)⁺: 504.2; found 504.2

### Example 8. 3-(5-amino-8-(1-methyl-6-oxo-1,6-dihydropyridin-3-yl)-2-((5-(pyrazin-2-yl)-1H-tetrazol-1-yl)methyl)-[1,2,4]triazolo[1,5-c]pyrimidin-7-yl)benzonitrile

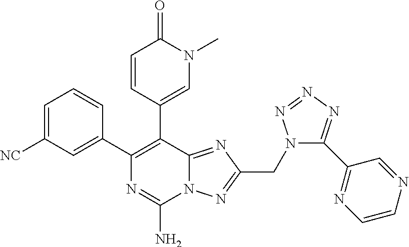 [SMILES: Cn1cc(-c2c(-c3cccc(C#N)c3)nc(N)n3nc(Cn4nnnc4-c4cnccn4)nc23)ccc1=O]

This compound was prepared using similar procedures as described for Example 4, 2-(1H-tetrazol-5-yl)pyrazine replacing 2-(1H-tetrazol-5-yl)pyridine. The product was purified by preparative LC-MS (pH 2, acetonitrile/water with TFA) to afford the product as a TFA salt. LCMS calculated for C₂₄H₁₈N₁₃O (M+H)⁺: 504.2; found 504.2

### Example 9. 3-(5-amino-8-(1-methyl-6-oxo-1,6-dihydropyridazin-3-yl)-2-((5-(pyridin-2-yl)-1H-tetrazol-1-yl)methyl)-[1,2,4]triazolo[1,5-c]pyrimidin-7-yl)benzonitrile

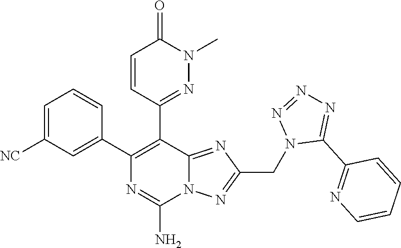 [SMILES: Cn1nc(-c2c(-c3cccc(C#N)c3)nc(N)n3nc(Cn4nnnc4-c4ccccn4)nc23)ccc1=O]

This compound was prepared using similar procedures as described for Example 4, with 2-methyl-6-(4,4,5,5-tetramethyl-1,3,2-dioxaborolan-2-yl)pyridazin-3(2H)-one replacing 1-methyl-5-(4,4,5,5-tetramethyl-1,3,2-dioxaborolan-2-yl)pyridin-2(1H)-one in step 1. The product was purified by preparative LC-MS (pH 2, acetonitrile/water with TFA) to afford the product as a TFA salt. LCMS calculated for C₂₄H₁₈N₁₃O (M+H)⁺: 504.2; found 504.2

### Example 10. 3-(5-amino-2-((5-(pyridin-2-yl)-1H-tetrazol-1-yl)methyl)-8-(pyridin-4-yl)-[1,2,4]triazolo[1,5-c]pyrimidin-7-yl)benzonitrile

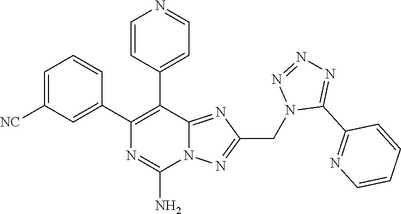 [SMILES: N#Cc1cccc(-c2nc(N)n3nc(Cn4nnnc4-c4ccccn4)nc3c2-c2ccncc2)c1]

This compound was prepared using similar procedures as described for Example 4, with pyridin-4-ylboronic acid replacing 1-methyl-5-(4,4,5,5-tetramethyl-1,3,2-dioxaborolan-2-yl)pyridin-2(1H)-one in step 1. The product was purified by preparative LC-MS (pH 2, acetonitrile/water with TFA) to afford the product as a TFA salt. LCMS calculated for C₂₄H₁₇N₁₂ (M+H)⁺: 473.2; found 473.2

### Example 11. 3-(5-amino-8-(2-(dimethylamino)pyridin-4-yl)-2-((5-(pyridin-2-yl)-1H-tetrazol-1-yl)methyl)-[1,2,4]triazolo[1,5-c]pyrimidin-7-yl)benzonitrile

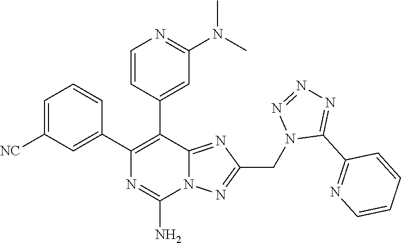 [SMILES: CN(C)c1cc(-c2c(-c3cccc(C#N)c3)nc(N)n3nc(Cn4nnnc4-c4ccccn4)nc23)ccn1]

This compound was prepared using similar procedures as described for Example 4, with (2-(dimethylamino)pyridin-4-yl)boronic acid replacing 1-methyl-5-(4,4,5,5-tetramethyl-1,3,2-dioxaborolan-2-yl)pyridin-2(1H)-one in step 1. The product was purified by preparative LC-MS (pH 2, acetonitrile/water with TFA) to afford the product as a TFA salt. LCMS calculated for C₂₆H₂₂N₁₃ (M+H)⁺: 515.2; found 515.2

### Example 12. 3-(5-amino-8-(2-aminopyridin-4-yl)-2-((5-(pyridin-2-yl)-1H-tetrazol-1-yl)methyl)-[1,2,4]triazolo[1,5-c]pyrimidin-7-yl)benzonitrile

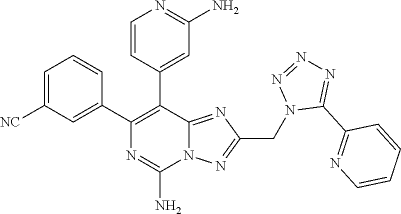 [SMILES: N#Cc1cccc(-c2nc(N)n3nc(Cn4nnnc4-c4ccccn4)nc3c2-c2ccnc(N)c2)c1]

This compound was prepared using similar procedures as described for Example 4, with (2-aminopyridin-4-yl)boronic acid replacing 1-methyl-5-(4,4,5,5-tetramethyl-1,3,2-dioxaborolan-2-yl)pyridin-2(1H)-one in step 1. The product was purified by preparative LC-MS (pH 2, acetonitrile/water with TFA) to afford the product as a TFA salt. LCMS calculated for C₂₄H₁₈N₁₃ (M+H)⁺: 488.2; found 488.2

### Example 13. 3-(5-amino-2-(2-fluoro-6-(pyridin-4-yl)benzyl)-8-(pyridin-4-yl)-[1,2,4]triazolo[1,5-c]pyrimidin-7-yl)benzonitrile

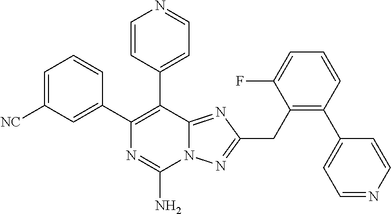 [SMILES: N#Cc1cccc(-c2nc(N)n3nc(Cc4c(F)cccc4-c4ccncc4)nc3c2-c2ccncc2)c1]

### Step 1: 3-(5-amino-2-(2-bromo-6-fluorobenzyl)-[1,2,4]triazolo[1,5-c]pyrimidin-7-yl)benzonitrile

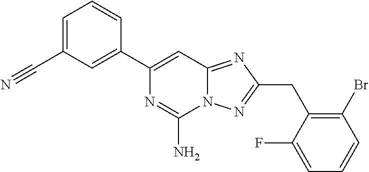 [SMILES: N#Cc1cccc(-c2cc3nc(Cc4c(F)cccc4Br)nn3c(N)n2)c1]

This compound was prepared using similar procedures as described for Example 1, step 2 with 2-(2-bromo-6-fluorophenyl)acetohydrazide replacing 2-hydroxyacetohydrazide. LCMS calculated for C₁₉H₁₃BrFN₆ (M+H)⁺: 423.0; found 423.0.

### Step 2: 3-(5-amino-8-bromo-2-(2-bromo-6-fluorobenzyl)-[1,2,4]triazolo[1,5-c]pyrimidin-7-yl)benzonitrile

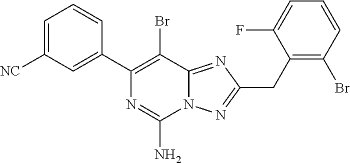 [SMILES: N#Cc1cccc(-c2nc(N)n3nc(Cc4c(F)cccc4Br)nc3c2Br)c1]

NBS (126 mg, 0.709 mmol) was added to a DMF (2.00 mL) solution of 3-(5-amino-2-(2-bromo-6-fluorobenzyl)-[1,2,4]triazolo[1,5-c]pyrimidin-7-yl)benzonitrile (300 mg, 0.709 mmol) at r.t. After stirring at rt for 1 h, diluted with water and the resulting precipitate was collected by filtration. The brown solid was dissolved in DCM and purified by flash chromatography on a silica gel column eluting with 0 to 50% EtOAc in DCM to afford the desired product. LCMS calculated for C₁₉H₁₂Br₂N₆ (M+H)⁺: 501.0; found 501.0.

### Step 3: 3-(5-amino-2-(2-fluoro-6-(pyridin-4-yl)benzyl)-8-(pyridin-4-yl)-[1,2,4]triazolo[1,5-c]pyrimidin-7-yl)benzonitrile

A mixture of 3-(5-amino-8-bromo-2-(2-bromo-6-fluorobenzyl)-[1,2,4]triazolo[1,5-c]pyrimidin-7-yl)benzonitrile (30 mg, 0.060 mmol) and pyridin-4-ylboronic acid (8.8 mg, 0.072 mmol), XPhos Pd G2 (4.7 mg, 6.0 μmol), and sodium carbonate (13.0 mg, 0.123 mmol) in 1,4-dioxane (2.0 mL) and water (0.20 mL) was stirred at 100° C. for 3 h. The product was purified by preparative LC-MS (pH 2, acetonitrile/water with TFA) to afford the product as a TFA salt. LCMS calculated for C₂₉H₂₀FN₈ (M+H)⁺: 499.2; found 499.2

### Example 14. 3-(5-amino-8-(2-aminopyridin-4-yl)-2-(2-(2-aminopyridin-4-yl)-6-fluorobenzyl)-[1,2,4]triazolo[1,5-c]pyrimidin-7-yl)benzonitrile

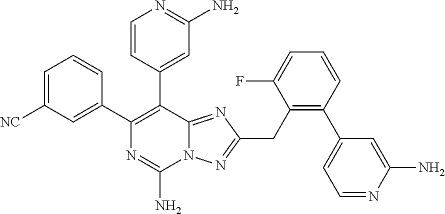 [SMILES: N#Cc1cccc(-c2nc(N)n3nc(Cc4c(F)cccc4-c4ccnc(N)c4)nc3c2-c2ccnc(N)c2)c1]

This compound was prepared using similar procedures as described for Example 13, with (2-aminopyridin-4-yl)boronic acid replacing pyridin-4-ylboronic acid in step 3. The product was purified by preparative LC-MS (pH 2, acetonitrile/water with TFA) to afford the product as a TFA salt. LCMS calculated for C₂₉H₂₂FN₁₀ (M+H)⁺: 529.2; found 529.3

### Example 15. 3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)benzonitrile

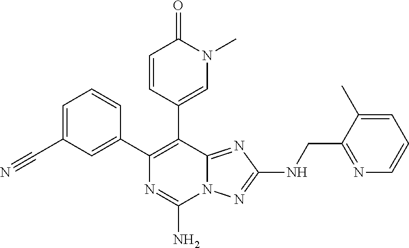 [SMILES: Cc1cccnc1CNc1nc2c(-c3ccc(=O)n(C)c3)c(-c3cccc(C#N)c3)nc(N)n2n1]

### Step 1:6-Chloro-N²,N²-bis(4-methoxybenzyl)pyrimidine-2,4-diamine

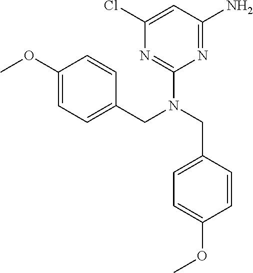 [SMILES: COc1ccc(CN(Cc2ccc(OC)cc2)c2nc(N)cc(Cl)n2)cc1]

To a solution of 2,6-dichloropyrimidin-4-amine (5.0 g, 31 mmol) in 2-propanol (31 mL) was added N,N-diisopropylethylamine (6.4 ml, 37 mmol) and bis(4-methoxybenzyl)amine (7.9 g, 31 mmol). The resulting solution was stirred at 100° C. for 16 h, cooled to r.t., diluted with water (100 mL), and extracted with EtOAc (100 mL). The organic layer was washed with water and brine, dried over anhydrous sodium sulfate, and concentrated to yield the crude product, which was used in the next step without further purification. LC-MS calculated for C₂H₂₂ClN₄O₂ (M+H)⁺: 385.1; found 385.1.

### Step 2:7-Chloro-N,N-bis(4-methoxybenzyl)-[1,2,4]triazolo[1,5-c]pyrimidine-2,5-diamine

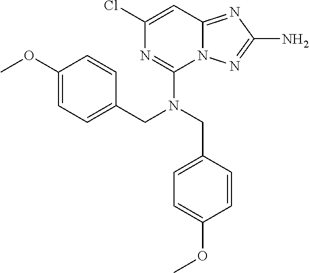 [SMILES: COc1ccc(CN(Cc2ccc(OC)cc2)c2nc(Cl)cc3nc(N)nn23)cc1]

O-ethyl carbonisothiocyanatidate (3.1 mL, 26 mmol) was added to a 1,4-dioxane (5.0 mL) solution of 6-chloro-N²,N²-bis(4-methoxybenzyl)pyrimidine-2,4-diamine (1.0 g, 2.6 mmol) at r.t. The reaction mixture was then stirred at 90° C. overnight, cooled to r.t., and concentrated. The resulting material was dissolved in methanol (12 mL) and ethanol (12 mL), and N,N-diisopropylethylamine (0.91 mL, 5.2 mmol) was added, followed by hydroxylamine hydrochoride (0.54 g, 7.8 mmol). The reaction mixture was stirred at 45° C. for 2 h, cooled to r.t., and concentrated. The resulting material was taken into EtOAc, washed with water, dried over anhydrous sodium sulfate, and concentrated. The crude material was then purified by silica gel chromatography eluting with 0% to 50% EtOAc in hexanes to afford the product. LC-MS calculated for C₂₁H₂₂ClN₆O2 (M+H)⁺: 425.1; found 425.2.

### Step 3: 3-(2-Amino-5-(bis(4-methoxybenzyl)amino)-[1,2,4]triazolo[1,5-c]pyrimidin-7-yl)benzonitrile

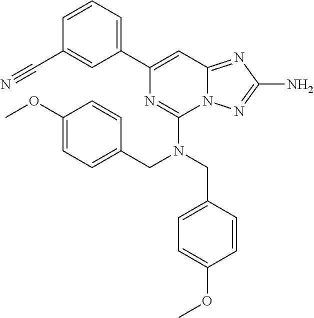 [SMILES: COc1ccc(CN(Cc2ccc(OC)cc2)c2nc(-c3cccc(C#N)c3)cc3nc(N)nn23)cc1]

Chloro(2-dicyclohexylphosphino-2′,4′,6′-tri-i-propyl-1,1′-biphenyl)(2′-amino-1,1′-biphenyl-2-yl) palladium(II) (330 mg, 0.42 mmol) was added to a mixture of (3-cyanophenyl)boronic acid (460 mg, 3.2 mmol), 7-chloro-N⁵,N-bis(4-methoxybenzyl)-[1,2,4]triazolo[1,5-c]pyrimidine-2,5-diamine (890 mg, 2.1 mmol), and sodium carbonate (890 mg, 8.4 mmol) in 1,4-dioxane (8.8 mL) and water (1.8 mL). The mixture was purged with N₂ and stirred at 95° C. overnight. The reaction mixture was then cooled to r.t., concentrated, and purified by silica gel chromatography eluting with 0% to 50% EtOAc in DCM to afford the desired product. LC-MS calculated for C₂₈H₂₆N₇O₂ (M+H)+. 492.2; found 492.2.

### Step 4: 3-(5-(bis(4-methoxybenzyl)amino)-2-(((3-methylpyridin-2-yl)methyl)amino)-[1,2,4]triazolo[1,5-c]pyrimidin-7-yl)benzonitrile

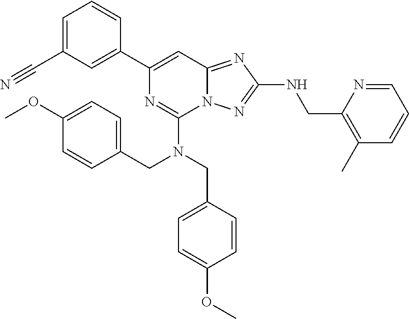 [SMILES: COc1ccc(CN(Cc2ccc(OC)cc2)c2nc(-c3cccc(C#N)c3)cc3nc(NCc4ncccc4C)nn23)cc1]

3-(2-amino-5-(bis(4-methoxybenzyl)amino)-[1,2,4]triazolo[1,5-c]pyrimidin-7-yl)benzonitrile (300 mg, 0.610 mmol), triethyl orthoformate (508 μl, 3.05 mmol), 3-methylpicolinaldehyde (148 mg, 1.221 mmol) were combined and EtOH (5 ml) was added. The suspension was heated at 120° C. overnight then cooled to room temperature, diluted with DCM (2 ml). To the solution was carefully added sodium tetrahydroborate (46.2 mg, 1.221 mmol). After stirring at room temperature for 1 h, the mixture was carefully quenched with saturated aqueous NH₄Cl, extracted with DCM and separated. The organic layer was dried over Na₂SO₄, filtered, evaporated. The residue was used in the next reaction without further purification. LCMS calculated for C₃₅H₃₃N₈O₂ (M+H)⁺: m/z=597.2; found 597.2.

### Step 5: 3-(5-(bis(4-methoxybenzyl)amino)-8-bromo-2-(((3-methylpyridin-2-yl)methyl)amino)-[1,2,4]triazolo[1,5-c]pyrimidin-7-yl)benzonitrile

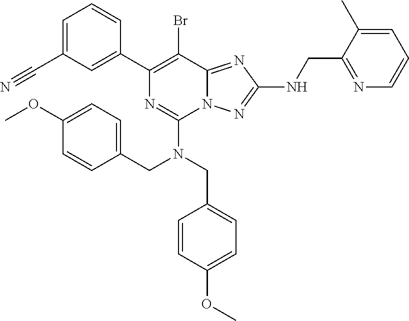 [SMILES: COc1ccc(CN(Cc2ccc(OC)cc2)c2nc(-c3cccc(C#N)c3)c(Br)c3nc(NCc4ncccc4C)nn23)cc1]

To a solution of 3-(5-(bis(4-methoxybenzyl)amino)-2-(((3-methylpyridin-2-yl)methyl)amino)-[1,2,4]triazolo[1,5-c]pyrimidin-7-yl)benzonitrile (300 mg, 0.503 mmol) in DCM (1 ml) at room temperature was added NBS (89 mg, 0.503 mmol). The mixture was stirred at room temperature for 1 h, and then quenched with saturated aqueous NaHCO₃, separated. The organic layer was dried over Na₂SO₄, filtered, evaporated. The residue was purified by column chromatography (10 to 50% AcOEt in hexane) to give the desired product. LCMS calculated for C₃₅H₃₂BrN₈O₂ (M+H)⁺: m/z=675.2; found 675.2.

### Step 6: 3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)benzonitrile

3-(5-(bis(4-methoxybenzyl)amino)-8-bromo-2-(((3-methylpyridin-2-yl)methyl)amino)-[1,2,4]triazolo[1,5-c]pyrimidin-7-yl)benzonitrile (30 mg, 0.044 mmol), 1-methyl-5-(4,4,5,5-tetramethyl-1,3,2-dioxaborolan-2-yl)pyridin-2(1H)-one (20.88 mg, 0.089 mmol), Xphos-G2 (3.49 mg, 4.44 μmol), sodium carbonate (9.41 mg, 0.089 mmol) were combined. To the mixture was added 1,4-dioxane (1 ml) and water (0.100 ml). The mixture was heated to 100° C. and stirred for 3 h, and then evaporated. To the residue was added TFA (1 ml) and the mixture was heated at 120° C. for 20 min. After cooled to room temperature, the mixture was diluted with acetonitrile, filtered and purified by prep-LC-MS (pH=2, MeCN/water with TFA) to give the desired product as a TFA salt. LCMS calculated for C₂₅H₂₂N₉O (M+H)⁺: m/z=464.2; found 464.2.

### Example 16. 3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)benzonitrile

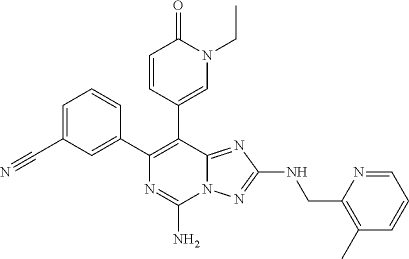 [SMILES: CCn1cc(-c2c(-c3cccc(C#N)c3)nc(N)n3nc(NCc4ncccc4C)nc23)ccc1=O]

This compound was prepared using similar procedures as described in Example 15 using 1-ethyl-5-(4,4,5,5-tetramethyl-1,3,2-dioxaborolan-2-yl)pyridin-2(1H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA to give the desired product as a TFA salt. LCMS calculated for C₂₆H₂₄N₉O (M+H)⁺: m/z=478.2; found 478.2.

### Example 17. 3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)benzonitrile

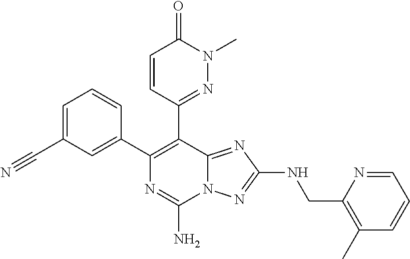 [SMILES: Cc1cccnc1CNc1nc2c(-c3ccc(=O)n(C)n3)c(-c3cccc(C#N)c3)nc(N)n2n1]

This compound was prepared using similar procedures as described in Example 15 using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA to give the desired product as a TFA salt. LCMS calculated for C₂₄H₂₁N₁₀O (M+H)⁺: m/z=465.2; found 465.2.

### Example 18. 3-(5-amino-8-(1-methyl-6-oxo-1,6-dihydropyridin-3-yl)-2-((3-methylpyridin-2-yl)methoxy)-[1,2,4]triazolo[1,5-c]pyrimidin-7-yl)benzonitrile

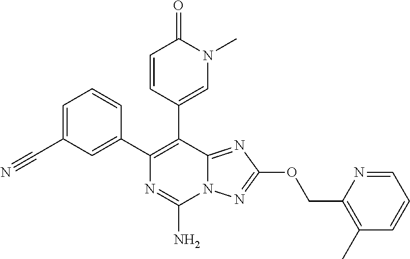 [SMILES: Cc1cccnc1COc1nc2c(-c3ccc(=O)n(C)c3)c(-c3cccc(C#N)c3)nc(N)n2n1]

### Step 1: 3-(5-(bis(4-methoxybenzyl)amino)-2-iodo-[1,2,4]triazolo[1,5-c]pyrimidin-7-yl)benzonitrile

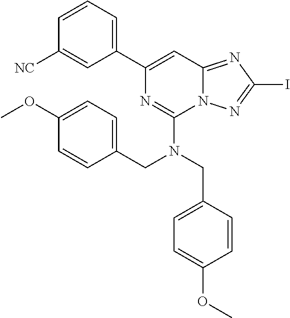 [SMILES: COc1ccc(CN(Cc2ccc(OC)cc2)c2nc(-c3cccc(C#N)c3)cc3nc(I)nn23)cc1]

To a CH₂Cl₂ (3.00 ml)/acetonitrile (3 ml) solution of 3-(2-amino-5-(bis(4-methoxybenzyl)amino)-[1,2,4]triazolo[1,5-c]pyrimidin-7-yl)benzonitrile (Example 15, step 3, 250 mg, 0.509 mmol) was added HI (57% in water, 201 μl, 1.526 mmol). The mixture was heated to 60° C., and then tert-butyl nitrite (134 μl, 1.017 mmol) was added. The reaction was heated at 60° C. for 20 min. After cooled to room temperature, 1N aqueous NH₄OH solution was added and the mixture was extracted three times with CH₂Cl₂. The combined organic layer was dried over sodium sulfate, filtered and the solvent was evaporated under reduced pressure. The residue was purified by silica gel chromatography (10 to 50% AcOEt in hexane) to give the desired product. LCMS calculated for C₂₈H₂₄IN₆O₂ (M+H)⁺: m/z=603.1; found 603.1.

### Step 2: 3-(5-(bis(4-methoxybenzyl)amino)-2-((3-methylpyridin-2-yl)methoxy)-[1,2,4]triazolo[1,5-c]pyrimidin-7-yl)benzonitrile

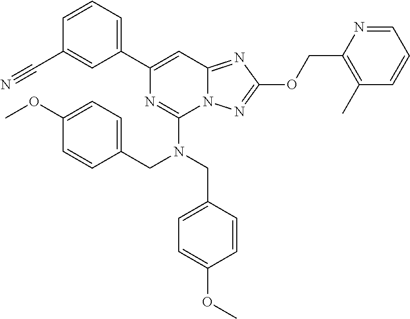 [SMILES: COc1ccc(CN(Cc2ccc(OC)cc2)c2nc(-c3cccc(C#N)c3)cc3nc(OCc4ncccc4C)nn23)cc1]

3-(5-(bis(4-methoxybenzyl)amino)-2-iodo-[1,2,4]triazolo[1,5-c]pyrimidin-7-yl)benzonitrile (250 mg, 0.415 mmol), (3-methylpyridin-2-yl)methanol (153 mg, 1.245 mmol) were combined and 1,4-dioxane (5 ml) was added. To the solution was added sodium hydride (60% in mineral oil, 41.5 mg, 1.037 mmol). The suspension was heated to 105° C. and stirred for 1 h, then cooled to room temperature and quenched with aqueous NH₄Cl, diluted with AcOEt, separated. The aqueous layer was extracted with AcOEt and the combined organic layer was washed with brine, separated, dried over N₂SO₄, filtered and evaporated. The residue was used for next reaction without further purification. LCMS calculated for C₃₅H₃₂N₇O₃ (M+H)⁺: m/z=598.2; found 598.2.

### Step 3: 3-(5-(bis(4-methoxybenzyl)amino)-8-bromo-2-((3-methylpyridin-2-yl)methoxy)-[1,2,4]triazolo[1,5-c]pyrimidin-7-yl)benzonitrile

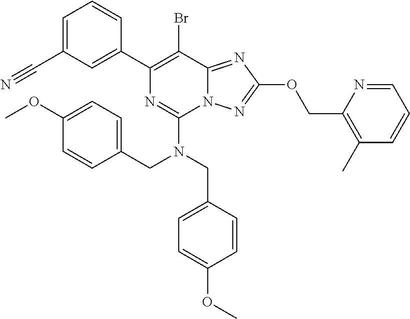 [SMILES: COc1ccc(CN(Cc2ccc(OC)cc2)c2nc(-c3cccc(C#N)c3)c(Br)c3nc(OCc4ncccc4C)nn23)cc1]

To a solution of 3-(5-(bis(4-methoxybenzyl)amino)-2-((3-methylpyridin-2-yl)methoxy)-[1,2,4]triazolo[1,5-c]pyrimidin-7-yl)benzonitrile (292 mg, 0.488 mmol) (300 mg, 0.503 mmol) in DCM (5 ml) at room temperature was added NBS (87 mg, 0.488 mmol). The mixture was stirred at room temperature for 1 h, and then quenched with saturated aqueous NaHCO₃, separated. The organic layer was dried over Na₂SO₄, filtered, evaporated. The residue was purified by column chromatography (10 to 50% AcOEt in hexane) to give the desired product. LCMS calculated for C₃₅H₃₁BrN₇O₃ (M+H)⁺: m/z=676.2; found 676.2.

### Step 4: 3-(5-amino-8-(1-methyl-6-oxo-1,6-dihydropyridin-3-yl)-2-((3-methylpyridin-2-yl)methoxy)-[1,2,4]triazolo[1,5-c]pyrimidin-7-yl)benzonitrile

3-(5-(bis(4-methoxybenzyl)amino)-8-bromo-2-((3-methylpyridin-2-yl)methoxy)-[1,2,4]triazolo[1,5-c]pyrimidin-7-yl)benzonitrile (20 mg, 0.030 mmol) (30 mg, 0.044 mmol), 1-methyl-5-(4,4,5,5-tetramethyl-1,3,2-dioxaborolan-2-yl)pyridin-2(1H)-one (13.90 mg, 0.059 mmol), Xphos-G2 (2.326 mg, 2.96 μmol) and sodium carbonate (6.27 mg, 0.059 mmol) were combined. To the mixture was added 1,4-Dioxane (1 ml) and Water (0.100 ml). The mixture was heated to 100° C. and stirred for 3 h, and then evaporated. To the residue was added TFA (1 ml) and the mixture was heated at 120° C. for 20 min. After cooled to room temperature, the mixture was diluted with acetonitrile, filtered and purified by prep-LC-MS (pH=2, MeCN/water with TFA) to give the desired product as a TFA salt. LCMS calculated for C₂₅H₂₁N₈O₂ (M+H)⁺: m/z=465.2; found 465.2.

### Example 19. 3-(5-amino-8-(1-methyl-6-oxo-1,6-dihydropyridazin-3-yl)-2-((3-methylpyridin-2-yl)methoxy)-[1,2,4]triazolo[1,5-c]pyrimidin-7-yl)benzonitrile

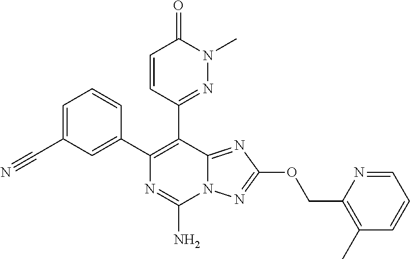 [SMILES: Cc1cccnc1COc1nc2c(-c3ccc(=O)n(C)n3)c(-c3cccc(C#N)c3)nc(N)n2n1]

This compound was prepared using similar procedures as described in Example 18 using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₄H₂₀N₉O₂ (M+H)⁺: m/z=466.2; found 466.2.

### Example 20. 3-(5-amino-2-((3-methylpyridin-2-yl)methoxy)-8-(3-methylpyridin-4-yl)-[1,2,4]triazolo[1,5-c]pyrimidin-7-yl)benzonitrile

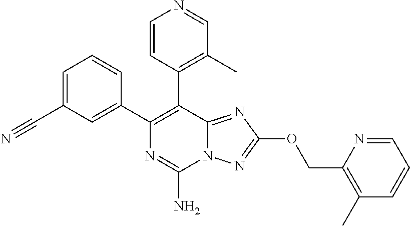 [SMILES: Cc1cnccc1-c1c(-c2cccc(C#N)c2)nc(N)n2nc(OCc3ncccc3C)nc12]

This compound was prepared using similar procedures as described in Example 18 using 3-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₅H₂₁N₈O (M+H)⁺: m/z=449.2; found 449.2.

### Example 21. 3-(5-amino-8-(2,6-dimethylpyridin-4-yl)-2-((3-methylpyridin-2-yl)methoxy)-[1,2,4]triazolo[1,5-c]pyrimidin-7-yl)benzonitrile

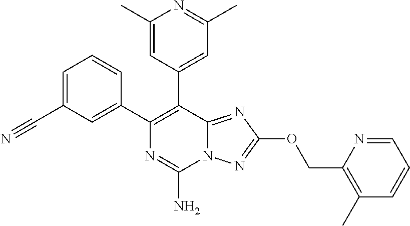 [SMILES: Cc1cc(-c2c(-c3cccc(C#N)c3)nc(N)n3nc(OCc4ncccc4C)nc23)cc(C)n1]

This compound was prepared using similar procedures as described in Example 18 using 2,6-dimethyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₆H₂₃N₈O (M+H)⁺: m/z=463.2; found 463.2.

### Example 22. 3-(5-amino-8-(1-ethyl-6-oxo-1,6-dihydropyridin-3-yl)-2-((3-methylpyridin-2-yl)methoxy)-[1,2,4]triazolo[1,5-c]pyrimidin-7-yl)benzonitrile

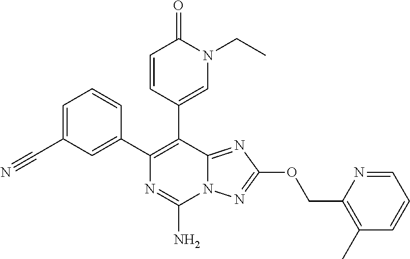 [SMILES: CCn1cc(-c2c(-c3cccc(C#N)c3)nc(N)n3nc(OCc4ncccc4C)nc23)ccc1=O]

This compound was prepared using similar procedures as described in Example 18 using 1-ethyl-5-(4,4,5,5-tetramethyl-1,3,2-dioxaborolan-2-yl)pyridin-2(1H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₆H₂₃N₈O₂ (M+H)⁺: m/z=479.2; found 479.2.

### Example 23. 3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile

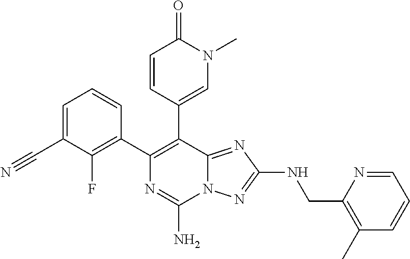 [SMILES: Cc1cccnc1CNc1nc2c(-c3ccc(=O)n(C)c3)c(-c3cccc(C#N)c3F)nc(N)n2n1]

### Step 1: 3-(2-amino-5-(bis(4-methoxybenzyl)amino)-[1,2,4]triazolo[1,5-c]pyrimidin-7-yl)-2-fluorobenzonitrile

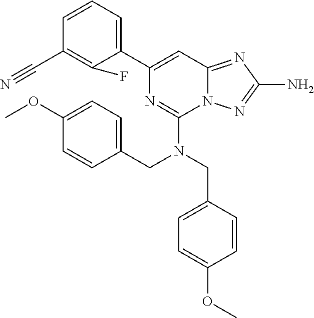 [SMILES: COc1ccc(CN(Cc2ccc(OC)cc2)c2nc(-c3cccc(C#N)c3F)cc3nc(N)nn23)cc1]

Chloro(2-dicyclohexylphosphino-2′,4′,6′-tri-i-propyl-1,1′-biphenyl)(2′-amino-1,1′-biphenyl-2-yl) palladium(II) (0.185 g, 0.235 mmol) was added to a mixture of (3-cyano-2-fluorophenyl)boronic acid (0.582 g, 3.53 mmol), 7-chloro-N5,N5-bis(4-methoxybenzyl)-[1,2,4]triazolo[1,5-c]pyrimidine-2,5-diamine (Example 15, step 2, 1 g, 2.354 mmol), sodium carbonate (0.499 g, 4.71 mmol) in 1,4-dioxane (13.08 ml) and water (2.62 ml). The mixture was purged with N₂ and heated at 110° C. overnight. The mixture was diluted with AcOEt and water and separated. The organic layer was washed with brine, dried over Na₂SO₄, concentrated and was purified by column chromatography (0 to 50% AcOEt in DCM). LCMS calculated for C₂₈H₂₅FN₇O₂ (M+H)⁺: m/z=510.2; found 510.2.

### Step 2: 3-(5-(bis(4-methoxybenzyl)amino)-2-(((3-methylpyridin-2-yl)methyl)amino)-[1,2,4]triazolo[1,5-c]pyrimidin-7-yl)-2-fluorobenzonitrile

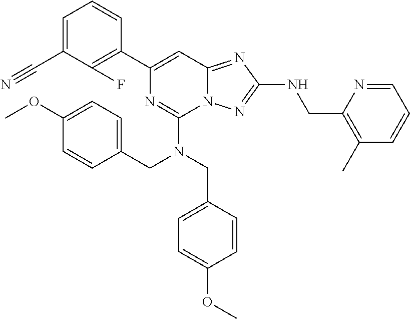 [SMILES: COc1ccc(CN(Cc2ccc(OC)cc2)c2nc(-c3cccc(C#N)c3F)cc3nc(NCc4ncccc4C)nn23)cc1]

3-(2-amino-5-(bis(4-methoxybenzyl)amino)-[1,2,4]triazolo[1,5-c]pyrimidin-7-yl)-2-fluorobenzonitrile (300 mg, 0.589 mmol) (300 mg, 0.610 mmol), triethyl orthoformate (490 μl, 2.94 mmol), 3-methylpicolinaldehyde (143 mg, 1.178 mmol) were combined and EtOH (5 ml) was added. The suspension was heated at 120° C. overnight. The mixture was then cooled to room temperature, diluted with DCM (2 ml). Sodium tetrahydroborate (46.2 mg, 1.221 mmol) was added to the solution carefully. After stirring at room temperature for 1 h, the mixture was carefully quenched with aqueous NH₄Cl, extracted with DCM and separated. The organic layer was dried over Na₂SO₄, filtered and evaporated. The residue was used in the next reaction without further purification. LCMS calculated for C₃₅H₃₂FN₈O₂ (M+H)⁺: m/z=615.2; found 615.2.

### Step 3: 3-(5-(bis(4-methoxybenzyl)amino)-8-bromo-2-(((3-methylpyridin-2-yl)methyl)amino)-[1,2,4]triazolo[1,5-c]pyrimidin-7-yl)-2-fluorobenzonitrile

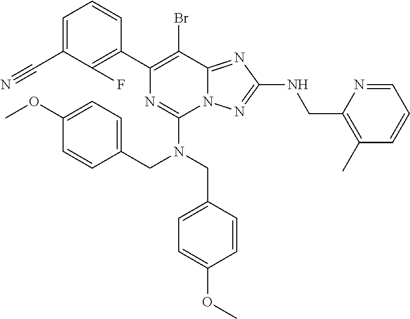 [SMILES: COc1ccc(CN(Cc2ccc(OC)cc2)c2nc(-c3cccc(C#N)c3F)c(Br)c3nc(NCc4ncccc4C)nn23)cc1]

To a solution of 3-(5-(bis(4-methoxybenzyl)amino)-2-(((3-methylpyridin-2-yl)methyl)amino)-[1,2,4]triazolo[1,5-c]pyrimidin-7-yl)-2-fluorobenzonitrile (300 mg, 0.488 mmol) in DCM (5 ml) at room temperature was added NBS (87 mg, 0.488 mmol). The mixture was stirred at room temperature for 1 h, then quenched with saturated aqueous NaHCO₃ and separated. The organic layer was dried over Na₂SO₄, filtered and evaporated. The residue was purified by column chromatography (10 to 50% AcOEt in hexane) to give the desired product. LCMS calculated for C₃₅H₃₁BrFN₈O₂ (M+H)⁺: m/z=693.2; found 693.2.

### Step 4: 3-(5-amino-8-(I-methyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile

3-(5-(bis(4-methoxybenzyl)amino)-8-bromo-2-(((3-methylpyridin-2-yl)methyl)amino)-[1,2,4]triazolo[1,5-c]pyrimidin-7-yl)-2-fluorobenzonitrile (20 mg, 0.029 mmol), 1-methyl-5-(4,4,5,5-tetramethyl-1,3,2-dioxaborolan-2-yl)pyridin-2(1H)-one (13.56 mg, 0.058 mmol), sodium carbonate (6.11 mg, 0.058 mmol) were combined. To the mixture was added 1,4-dioxane (1 ml) and water (0.100 ml). The mixture was heated to 100° C. and stirred for 3 h, and then evaporated. To the residue was added TFA (1 ml) and the mixture was heated at 120° C. for 20 min. After cooled to room temperature, the mixture was diluted with acetonitrile, filtered and purified by prep-LC-MS (pH=2, MeCN/water with TFA) to give the desired product as a TFA salt. LCMS calculated for C₂₅H₂₁FN₉O (M+H)⁺: m/z=482.2; found 482.2.

### Example 24. 3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile

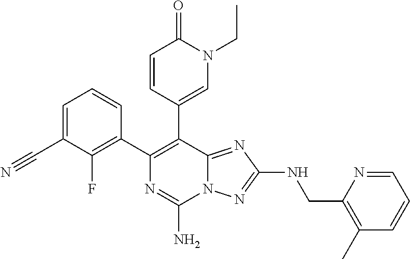 [SMILES: CCn1cc(-c2c(-c3cccc(C#N)c3F)nc(N)n3nc(NCc4ncccc4C)nc23)ccc1=O]

This compound was prepared using similar procedures as described in Example 23 using 1-ethyl-5-(4,4,5,5-tetramethyl-1,3,2-dioxaborolan-2-yl)pyridin-2(1H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₆H₂₃FN₉O (M+H)⁺: m/z=496.2; found 496.2.

### Example 25. 3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)-2-fluorobenzonitrile

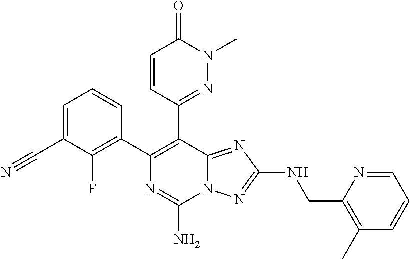 [SMILES: Cc1cccnc1CNc1nc2c(-c3ccc(=O)n(C)n3)c(-c3cccc(C#N)c3F)nc(N)n2n1]

This compound was prepared using similar procedures as described in Example 23 using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₄H₂₀FN₁₀O (M+H)⁺: m/z=483.2; found 483.2.

### Example 26. 3-(5-amino-8-(2-methoxy-6-methylpyridin-4-yl)-2-(((3-methylpyridin-2-yl)methyl)amino)-[1,2,4]triazolo[1,5-c]pyrimidin-7-yl)-2-fluorobenzonitrile

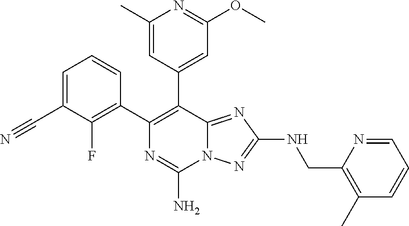 [SMILES: COc1cc(-c2c(-c3cccc(C#N)c3F)nc(N)n3nc(NCc4ncccc4C)nc23)cc(C)n1]

This compound was prepared using similar procedures as described in Example 23 using 2-methoxy-6-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₆H₂₃FN₉O (M+H)⁺: m/z=496.2; found 496.2.

### Example 27. 3-(5-amino-8-(1-methyl-6-oxo-1,6-dihydropyridazin-3-yl)-2-(pyridin-2-ylmethyl)-[1,2,4]triazolo[1,5-c]pyrimidin-7-yl)benzonitrile

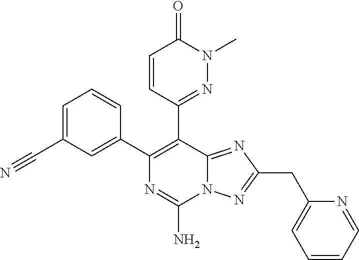 [SMILES: Cn1nc(-c2c(-c3cccc(C#N)c3)nc(N)n3nc(Cc4ccccn4)nc23)ccc1=O]

### Step 1: 2-(Pyridin-2-yl)acetohydrazide

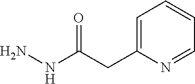 [SMILES: NNC(=O)Cc1ccccn1]

Hydrazine (4.15 mL, 132 mmol) was added to a ethanol (66 mL) solution of methyl 2-(pyridin-2-yl)acetate (10 g, 66.2 mmol) at r.t. The mixture was heated and stirred at 85° C. for 4 h, and then cooled to r.t. White solid was formed upon standing, which was collected via filtration and used in next step without further purification. LCMS calculated for C₇H₁₀N₃O (M+H)⁺: 152.1. Found: 152.0.

### Step 2:3-(5-Amino-2-(pyridin-2-ylmethyl)-[1,2,4]triazolo[1,5-c]pyrimidin-7-yl)benzonitrile

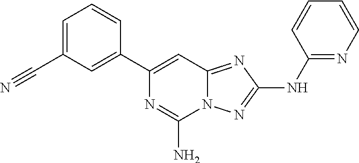 [SMILES: N#Cc1cccc(-c2cc3nc(Nc4ccccn4)nn3c(N)n2)c1]

2-(pyridin-2-yl)acetohydrazide (2.62 g, 17.34 mmol) was added to a ethanol (35 mL) solution of 3-(2-amino-6-chloropyrimidin-4-yl)benzonitrile (Example 1, Step 1, 4.00 g, 17.34 mmol) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₈H₁₄N₇ (M+H)⁺: 328.1; found 328.1.

### Step 3: 3-(5-Amino-8-bromo-2-(pyridin-2-ylmethyl)-[1,2,4]triazolo[1,5-c]pyrimidin-7-yl)benzonitrile

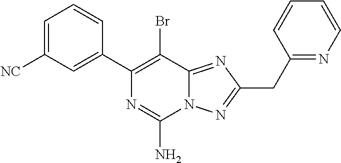 [SMILES: N#Cc1cccc(-c2nc(N)n3nc(Cc4ccccn4)nc3c2Br)c1]

To a mixture of 3-(5-amino-2-(pyridin-2-ylmethyl)-[1,2,4]triazolo[1,5-c]pyrimidin-7-yl)benzonitrile (2 g, 6.11 mmol) in DMF (12 mL) at −30° C. was added NBS (1.09 g, 6.11 mmol) portion-wise. The reaction mixture was allowed to slowly warm to 0° C., resulting a homogenous solution. After stirring at 0° C. for 1 h, the reaction mixture was diluted with saturated NaHCO₃ solution and the resulting solid was collected by filtration. The solid was then purified by flash chromatography on a silica gel column eluting with 0 to 10% MeOH in DCM to afford the desired product. LCMS calculated for C₁₈H₁₃BrN₇ (M+H)⁺: 406.0; found 406.0.

### Step 4: 3-(5-amino-8-(I-methyl-6-oxo-1,6-dihydropyridazin-3-yl)-2-(pyridin-2-ylmethyl)-[1,2,4]triazolo[1,5-c]pyrimidin-7-yl)benzonitrile

3-(5-amino-8-bromo-2-(pyridin-2-ylmethyl)-[1,2,4]triazolo[1,5-c]pyrimidin-7-yl)benzonitrile (15 mg, 0.037 mmol), 2-methyl-6-(4,4,5,5-tetramethyl-1,3,2-dioxaborolan-2-yl)pyridazin-3(2H)-one (17.43 mg, 0.074 mmol), sodium carbonate (7.48 mg, 0.070 mmol) and Xphos-G2 (1.387 mg, 1.763 μmol) were combined. To the mixture was added 1,4-dioxane (1 ml) and water (0.100 ml). The mixture was heated to 100° C. and stirred for 3 h, and then cooled to room temperature, diluted with acetonitrile and TFA, filtered and purified by prep-LC-MS (pH=2, MeCN/water with TFA) to the desired product as a TFA salt. LCMS calculated for C₂₃H₁₈N₉O (M+H)⁺: m/z=436.2; found 436.2.

### Example 28. 3-(5-amino-2-((3-fluoropyridin-2-yl)methoxy)-8-(pyrimidin-4-yl)-[1,2,4]triazolo[1,5-c]pyrimidin-7-yl)benzonitrile

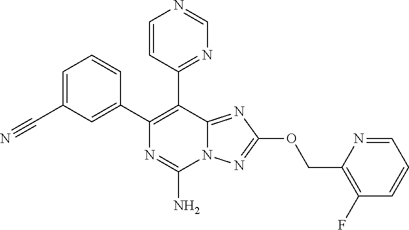 [SMILES: N#Cc1cccc(-c2nc(N)n3nc(OCc4ncccc4F)nc3c2-c2ccncn2)c1]

### Step 1: 3-(5-(bis(4-methoxybenzyl)amino)-2-((3-fluoropyridin-2-yl)methoxy)-[1,2,4]triazolo[1,5-c]pyrimidin-7-yl)benzonitrile

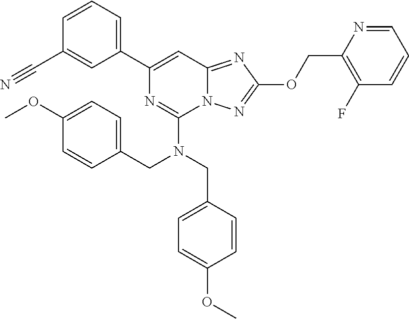 [SMILES: COc1ccc(CN(Cc2ccc(OC)cc2)c2nc(-c3cccc(C#N)c3)cc3nc(OCc4ncccc4F)nn23)cc1]

3-(5-(bis(4-methoxybenzyl)amino)-2-iodo-[1,2,4]triazolo[1,5-c]pyrimidin-7-yl)benzonitrile (250 mg, 0.415 mmol), (3-fluoropyridin-2-yl)methanol (158 mg, 1.245 mmol) were combined and 1,4-dioxane (5 ml) was added. To the solution was added sodium hydride (60% in mineral oil, 41.5 mg, 1.037 mmol). The suspension was heated to 105° C. and stirred for 1 h, then cooled to room temperature and quenched with aqueous NH₄Cl, diluted with AcOEt, separated. The aqueous layer was extracted with AcOEt and the combined organic layer was washed with brine, separated, dried over Na₂SO₄, filtered and evaporated. The residue was used for next reaction without further purification. LCMS calculated for C₃₄H₂₉FN₇O₃ (M+H)⁺: m/z=602.2; found 602.2.

### Step 2: 3-(5-(bis(4-methoxybenzyl)amino)-8-bromo-2-((3-fluoropyridin-2-yl)methoxy)-[1,2,4]triazolo[1,5-c]pyrimidin-7-yl)benzonitrile

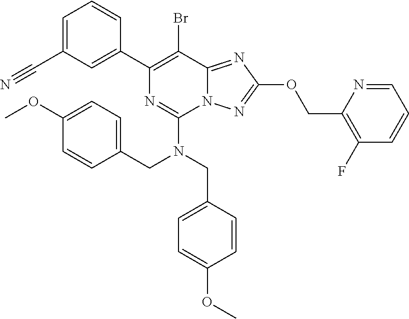 [SMILES: COc1ccc(CN(Cc2ccc(OC)cc2)c2nc(-c3cccc(C#N)c3)c(Br)c3nc(OCc4ncccc4F)nn23)cc1]

To a solution of 3-(5-(bis(4-methoxybenzyl)amino)-2-((3-fluoropyridin-2-yl)methoxy)-[1,2,4]triazolo[1,5-c]pyrimidin-7-yl)benzonitrile (294 mg, 0.488 mmol) (300 mg, 0.503 mmol) in DCM (5 ml) at room temperature was added NBS (87 mg, 0.488 mmol). The mixture was stirred at room temperature for 1 h, and then quenched with saturated aqueous NaHCO₃, separated. The organic layer was dried over Na₂SO₄, filtered and evaporated. The residue was purified by column chromatography (10 to 50% AcOEt in hexane) to give the desired product. LCMS calculated for C₃₄H₂₈BrFN₇O₃ (M+H)⁺: m/z=680.2; found 680.2.

### Step 3: 3-(5-amino-2-((3-fluoropyridin-2-yl)methoxy)-8-(pyrimidin-4-yl)-[1,2,4]triazolo[1,5-c]pyrimidin-7-yl)benzonitrile

3-(5-(bis(4-methoxybenzyl)amino)-8-bromo-2-((3-fluoropyridin-2-yl)methoxy)-[1,2,4]triazolo[1,5-c]pyrimidin-7-yl)benzonitrile (30 mg, 0.044 mmol), tetrakis (10.19 mg, 8.82 μmol), 4-(tributylstannyl)pyrimidine (24.41 mg, 0.066 mmol) were combined and 1,4-dioxane (1 ml) was added. The mixture was heated to 110° C. and stirred for 3 h, and then evaporated. To the residue was added TFA (1 ml) and the mixture was heated at 120° C. for 20 min. After cooled to room temperature, the mixture was diluted with acetonitrile, filtered and purified by prep-LC-MS (pH=2, MeCN/water with TFA) to give the product as a TFA salt. LCMS calculated for C₂₂H₁₅FN₉O (M+H)⁺: m/z=440.2; found 440.2.

### Example 29. 3-(5-amino-2-((3-fluoropyridin-2-yl)methoxy)-8-(1-methyl-6-oxo-1,6-dihydropyridin-3-yl)-[1,2,4]triazolo[1,5-c]pyrimidin-7-yl)benzonitrile

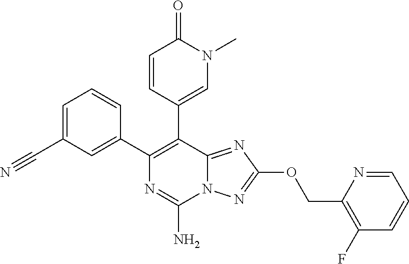 [SMILES: Cn1cc(-c2c(-c3cccc(C#N)c3)nc(N)n3nc(OCc4ncccc4F)nc23)ccc1=O]

3-(5-(bis(4-methoxybenzyl)amino)-8-bromo-2-((3-fluoropyridin-2-yl)methoxy)-[1,2,4]triazolo[1,5-c]pyrimidin-7-yl)benzonitrile (12 mg, 0.018 mmol), 1-methyl-5-(4,4,5,5-tetramethyl-1,3,2-dioxaborolan-2-yl)pyridin-2(1H)-one (8.29 mg, 0.035 mmol), sodium carbonate (3.74 mg, 0.035 mmol), Xphos-G2 (1.387 mg, 1.763 μmol) were combined. To the mixture was added 1,4-dioxane (1 ml) and water (0.100 ml). The mixture was heated to 100° C. and stirred for 3 h, and then evaporated. To the residue was added TFA (1 ml) and the mixture was heated at 120° C. for 20 min. After cooled to room temperature, the mixture was diluted with acetonitrile, filtered and purified by prep-LC-MS (pH=2, MeCN/water with TFA) to the desired product as a TFA salt. LCMS calculated for C₂₄H₁₈FN₈O₂ (M+H)⁺: m/z=469.2; found 469.2.

### Example 30. 3-(5-amino-2-((3-fluoropyridin-2-yl)methoxy)-8-(1-methyl-6-oxo-1,6-dihydropyridazin-3-yl)-[1,2,4]triazolo[1,5-c]pyrimidin-7-yl)benzonitrile

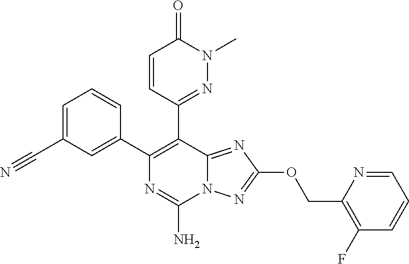 [SMILES: Cn1nc(-c2c(-c3cccc(C#N)c3)nc(N)n3nc(OCc4ncccc4F)nc23)ccc1=O]

This compound was prepared using similar procedures as described in Example 29 using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₃H₁₇FN₉O₂ (M+H)⁺: m/z=470.2; found 470.2.

### Example 31. 3-(5-amino-2-((3-fluoropyridin-2-yl)methoxy)-8-(3-methylpyridin-4-yl)-[1,2,4]triazolo[1,5-c]pyrimidin-7-yl)benzonitrile

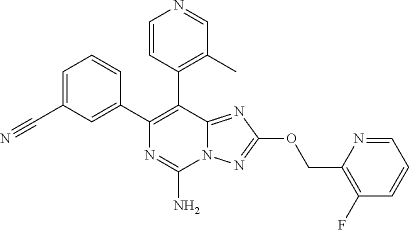 [SMILES: Cc1cnccc1-c1c(-c2cccc(C#N)c2)nc(N)n2nc(OCc3ncccc3F)nc12]

This compound is prepared using similar procedures as described in Example 29, using 3-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material is purified by prep-LC-MS (pH=2, MeCN/water with TFA) to give the desired product as a TFA salt. LCMS calculated for C₂₄H₁₈FN₈O (M+H)⁺: m/z=453.2.

### Example 32. 3-(5-amino-8-(2,6-dimethylpyridin-4-yl)-2-((3-fluoropyridin-2-yl)methoxy)-[1,2,4]triazolo[1,5-c]pyrimidin-7-yl)benzonitrile

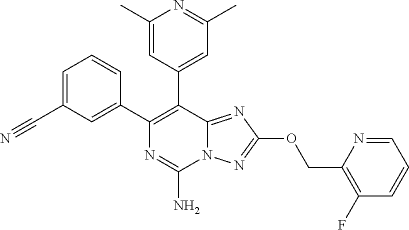 [SMILES: Cc1cc(-c2c(-c3cccc(C#N)c3)nc(N)n3nc(OCc4ncccc4F)nc23)cc(C)n1]

This compound was prepared using similar procedures as described in Example 29 using 2,6-dimethyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₅H₂₀FN₈O (M+H)⁺: m/z=467.2; found 467.2.

### Example 33. 3-(5-amino-2-((3-fluoropyridin-2-yl)methoxy)-8-(2-methoxy-6-methylpyridin-4-yl)-[1,2,4]triazolo[1,5-c]pyrimidin-7-yl)benzonitrile

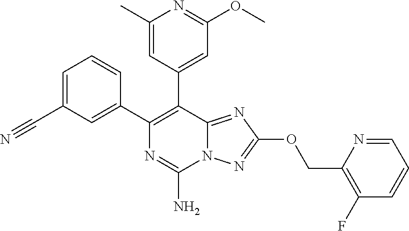 [SMILES: COc1cc(-c2c(-c3cccc(C#N)c3)nc(N)n3nc(OCc4ncccc4F)nc23)cc(C)n1]

This compound was prepared using similar procedures as described in Example 29 using 2-methoxy-6-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₅H₂₀FN₈O₂ (M+H)⁺: m/z=483.2; found 483.2.

### Example 34. 3-(5-amino-2-(pyridin-2-ylamino)-8-(pyrimidin-4-yl)-[1,2,4]triazolo[1,5-c]pyrimidin-7-yl)benzonitrile

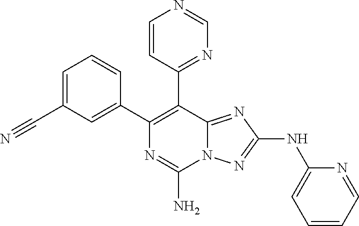 [SMILES: N#Cc1cccc(-c2nc(N)n3nc(Nc4ccccn4)nc3c2-c2ccncn2)c1]

### Step 1: 3-(5-(bis(4-methoxybenzyl)amino)-2-(pyridin-2-ylamino)-[1,2,4]triazolo[1,5-c]pyrimidin-7-yl)benzonitrile

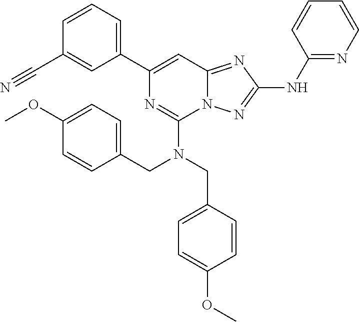 [SMILES: COc1ccc(CN(Cc2ccc(OC)cc2)c2nc(-c3cccc(C#N)c3)cc3nc(Nc4ccccn4)nn23)cc1]

3-(2-amino-5-(bis(4-methoxybenzyl)amino)-[1,2,4]triazolo[1,5-c]pyrimidin-7-yl)benzonitrile (Example 15, step 3, 200 mg, 0.407 mmol), 2-bromopyridine (79 μl, 0.814 mmol), sodium 2-methylpropan-2-olate (78 mg, 0.814 mmol), XPhos-G2 (64.0 mg, 0.081 mmol) were combined and t-BuOH (5 ml) was added. The mixture was heated at 70° C. overnight, then quenched with aqueous NH₄Cl solution, diluted with AcOEt and separated. The organic layer was washed by brine, dried over Na₂SO₄, filtered and evaporated. The residue was used for next reaction without further purification. LCMS calculated for C₃₃H₂₉N₈O₂ (M+H)⁺: m/z=569.2; found 569.2.

### Step 2: 3-(5-(bis(4-methoxybenzyl)amino)-8-bromo-2-(pyridin-2-ylamino)-[1,2,4]triazolo[1,5-c]pyrimidin-7-yl)benzonitrile

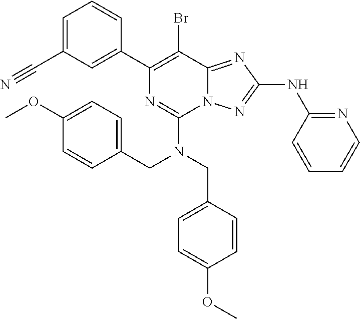 [SMILES: COc1ccc(CN(Cc2ccc(OC)cc2)c2nc(-c3cccc(C#N)c3)c(Br)c3nc(Nc4ccccn4)nn23)cc1]

To a solution of 3-(5-(bis(4-methoxybenzyl)amino)-2-(pyridin-2-ylamino)-[1,2,4]triazolo[1,5-c]pyrimidin-7-yl)benzonitrile (278 mg, 0.488 mmol) (300 mg, 0.503 mmol) in DCM (5 ml) at room temperature was added NBS (87 mg, 0.488 mmol). The mixture was stirred at room temperature for 1 h, then quenched with saturated aqueous NaHCO₃ and separated. The organic layer was dried over Na₂SO₄, filtered, evaporated. The residue was purified by column chromatography (10 to 60% AcOEt in hexane) to give the desired product. LCMS calculated for C₃₃H₂₈BrN₈O₂ (M+H)⁺: m/z=647.2; found 647.2.

### Step 3: 3-(5-amino-2-(pyridin-2-ylamino)-8-(pyrimidin-4-yl)-[1,2,4]triazolo[1,5-c]pyrimidin-7-yl)benzonitrile

3-(5-(bis(4-methoxybenzyl)amino)-8-bromo-2-(pyridin-2-ylamino)-[1,2,4]triazolo[1,5-c]pyrimidin-7-yl)benzonitrile (30 mg, 0.046 mmol), tetrakis (10.71 mg, 9.27 μmol), 4-(tributylstannyl)pyrimidine (25.7 mg, 0.069 mmol) were combined and 1,4-dioxane (1 ml) was added. The mixture was heated to 110° C. and stirred for 3 h, and then evaporated. To the residue was added TFA (1 ml) and the mixture was heated at 120° C. for 20 min. After cooled to room temperature, the mixture was diluted with acetonitrile, filtered and purified by prep-LC-MS (pH=2, MeCN/water with TFA) to give the product as a TFA salt. LCMS calculated for C₂₁H₅N₁₀ (M+H)⁺: m/z=407.2; found 407.2.

### Example 35. 3-(5-amino-8-(1-methyl-6-oxo-1,6-dihydropyridin-3-yl)-2-(pyridin-2-ylamino)-[1,2,4]triazolo[1,5-c]pyrimidin-7-yl)benzonitrile

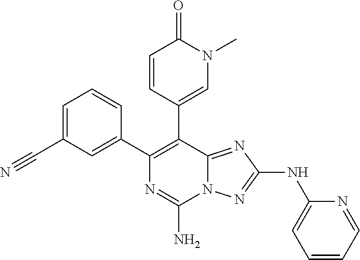 [SMILES: Cn1cc(-c2c(-c3cccc(C#N)c3)nc(N)n3nc(Nc4ccccn4)nc23)ccc1=O]

3-(5-(bis(4-methoxybenzyl)amino)-8-bromo-2-(pyridin-2-ylamino)-[1,2,4]triazolo[1,5-c]pyrimidin-7-yl)benzonitrile (20 mg, 0.031 mmol), 1-methyl-5-(4,4,5,5-tetramethyl-1,3,2-dioxaborolan-2-yl)pyridin-2(1H)-one (14.52 mg, 0.062 mmol), sodium carbonate (6.55 mg, 0.062 mmol), Xphos-G2 (2.430 mg, 3.09 μmol) are combined, and to the mixture is added 1,4-dioxane (1 ml) and water (0.100 ml). The mixture is heated at 100° C. for 3 h, and then evaporated. To the residue is added TFA (1 ml) and the mixture is heated at 120° C. for 20 min. After cooling to room temperature, the mixture is diluted with acetonitrile, filtered and purified by prep-LC-MS (pH=2, MeCN/water with TFA) to afford the desired product as a TFA salt. LCMS calculated for C₂₃H₁₈N₉O (M+H)⁺: m/z=436.2.

### Example 36. 3-(5-amino-8-(1-methyl-6-oxo-1,6-dihydropyridazin-3-yl)-2-(pyridin-2-ylamino)-[1,2,4]triazolo[1,5-c]pyrimidin-7-yl)benzonitrile

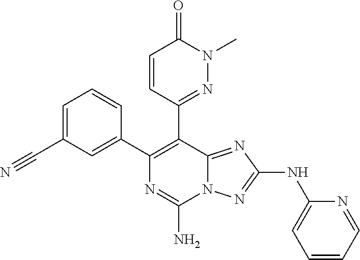 [SMILES: Cn1nc(-c2c(-c3cccc(C#N)c3)nc(N)n3nc(Nc4ccccn4)nc23)ccc1=O]

This compound is prepared using similar procedures as described in Example 35, using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one. The final material is purified by prep-LC-MS (pH=2, MeCN/water with TFA) to give the desired product as a TFA salt. LCMS calculated for C₂₂H₁₇N₁₀O (M+H)⁺: m/z=437.2.

### Example 37. 3-(5-amino-8-(3-methylpyridin-4-yl)-2-(pyridin-2-ylamino)-[1,2,4]triazolo[1,5-c]pyrimidin-7-yl)benzonitrile

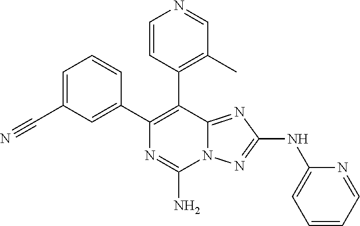 [SMILES: Cc1cnccc1-c1c(-c2cccc(C#N)c2)nc(N)n2nc(Nc3ccccn3)nc12]

This compound is prepared using similar procedures as described in Example 35, using 3-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material is purified by prep-LC-MS (pH=2, MeCN/water with TFA) to give the desired product as a TFA salt. LCMS calculated for C₂₃H₁₈N9 (M+H)⁺: m/z=420.2.

### Example 38. 3-(5-amino-8-(2,6-dimethylpyridin-4-yl)-2-(pyridin-2-ylamino)-[1,2,4]triazolo[1,5-c]pyrimidin-7-yl)benzonitrile

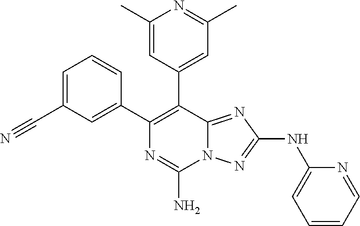 [SMILES: Cc1cc(-c2c(-c3cccc(C#N)c3)nc(N)n3nc(Nc4ccccn4)nc23)cc(C)n1]

This compound is prepared using similar procedures as described in Example 35, using 2,6-dimethyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material is purified by prep-LC-MS (pH=2, MeCN/water with TFA) to give the desired product as a TFA salt. LCMS calculated for C₂₄H₂₀N₉ (M+H)⁺: m/z=434.2.

### Example 39. 3-(5-amino-8-(2-methoxy-6-methylpyridin-4-yl)-2-(pyridin-2-ylamino)-[1,2,4]triazolo[1,5-c]pyrimidin-7-yl)benzonitrile

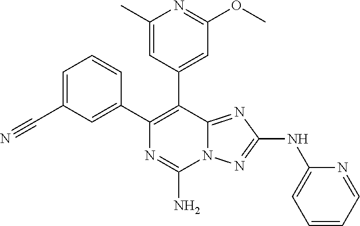 [SMILES: COc1cc(-c2c(-c3cccc(C#N)c3)nc(N)n3nc(Nc4ccccn4)nc23)cc(C)n1]

This compound is prepared using similar procedures as described in Example 35, using 2-methoxy-6-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material is purified by prep-LC-MS (pH=2, MeCN/water with TFA) to give the desired product as a TFA salt. LCMS calculated for C₂₄H₂₀N₉O (M+H)⁺: m/z=450.2.

### Example 40. 3-(5-amino-2-((6-methylpyridin-2-yl)amino)-8-(pyrimidin-4-yl)-[1,2,4]triazolo[1,5-c]pyrimidin-7-yl)benzonitrile

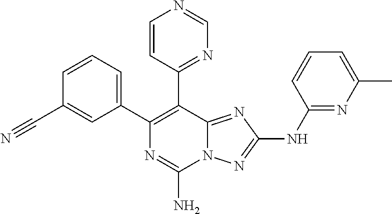 [SMILES: Cc1cccc(Nc2nc3c(-c4ccncn4)c(-c4cccc(C#N)c4)nc(N)n3n2)n1]

This compound was prepared using similar procedures as described in Example 34 using 2-bromo-6-methylpyridine in place of 2-bromopyridine. The final material was purified by prep-LC-MS (pH=2, MeCN/water with TFA) to give the desired product as a TFA salt. LCMS calculated for C₂₂H₁₇N₁₀ (M+H)⁺: m/z=421.2; found 421.2.

### Example 41. 3-(5-amino-2-((pyridin-2-yloxy)methyl)-8-(pyrimidin-4-yl)-[1,2,4]triazolo[1,5-c]pyrimidin-7-yl)benzonitrile

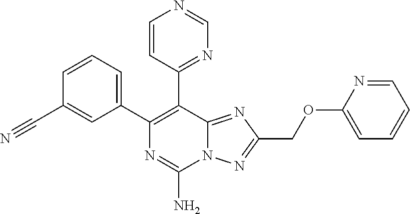 [SMILES: N#Cc1cccc(-c2nc(N)n3nc(COc4ccccn4)nc3c2-c2ccncn2)c1]

To a 1,4-dioxane (1 ml) solution of 3-(5-amino-2-(hydroxymethyl)-8-(pyrimidin-4-yl)-[1,2,4]triazolo[1,5-c]pyrimidin-7-yl)benzonitrile (Example 1, step 4, 15 mg, 0.044 mmol) and 2-fluoropyridine (0.011 ml, 0.131 mmol) was added sodium hydride (60% in mineral oil, 3.48 mg, 0.087 mmol). The mixture was heated at 60° C. for 3 h, and then cooled to room temperature, diluted with acetonitrile and TFA, filtered and purified by prep-LC-MS (pH=2, MeCN/water with TFA) to the desired product as a TFA salt. LCMS calculated for C₂₂H₁₆N₉O (M+H)⁺: m/z=422.2; found 422.2.

### Example 42. 2-((5-amino-7-(3-cyanophenyl)-8-(pyrimidin-4-yl)-[1,2,4]triazolo[1,5-c]pyrimidin-2-yl)methoxy)nicotinonitrile

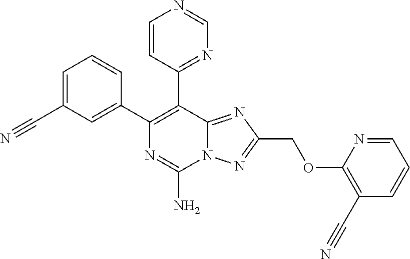 [SMILES: N#Cc1cccc(-c2nc(N)n3nc(COc4ncccc4C#N)nc3c2-c2ccncn2)c1]

### Step 1: 2-((5-amino-7-(3-cyanophenyl)-[1,2,4]triazolo[1,5-c]pyrimidin-2-yl)methoxy)nicotinonitrile

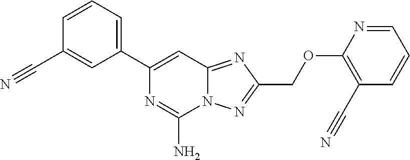 [SMILES: N#Cc1cccc(-c2cc3nc(COc4ncccc4C#N)nn3c(N)n2)c1]

To a 1,4-dioxane (5 ml) solution of 3-(5-amino-2-(hydroxymethyl)-[1,2,4]triazolo[1,5-c]pyrimidin-7-yl)benzonitrile (Example 1, step 2, 100 mg, 0.376 mmol) and 2-fluoronicotinonitrile (138 mg, 1.127 mmol) was added sodium hydride (60% in mineral oil, 30.0 mg, 0.751 mmol). The mixture was heated at 60° C. for 3 h, and then cooled to room temperature, quenched with aqueous NH₄Cl, diluted with DCM, separated. The organic layer was dried over Na₂SO₄, filtered and evaporated. The residue was used for next reaction without further purification. LCMS calculated for C₁₉H₁₃N₈O (M+H)⁺: m/z=369.2; found 369.2.

### Step 2: 2-((5-amino-8-bromo-7-(3-cyanophenyl)-[1,2,4]triazolo[1,5-c]pyrimidin-2-yl)methoxy)nicotinonitrile

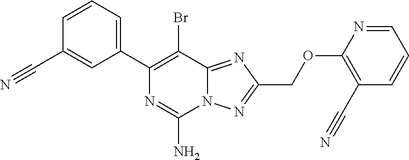 [SMILES: N#Cc1cccc(-c2nc(N)n3nc(COc4ncccc4C#N)nc3c2Br)c1]

To a suspension of 2-((5-amino-7-(3-cyanophenyl)-[1,2,4]triazolo[1,5-c]pyrimidin-2-yl)methoxy)nicotinonitrile (185 mg, 0.503 mmol) in DCM (1 ml) at room temperature was added NBS (89 mg, 0.503 mmol). The mixture was stirred at room temperature for 1 h. After cooled to room temperature, the mixture was quenched with saturated aqueous NaHCO₃, diluted with DCM and separated. The organic layer was dried over Na₂SO₄, filtered and evaporated. The residue was purified by column chromatography (0 to 10% MeOH in DCM) to give the desired product. LCMS calculated for C₁₉H₁₂BrN₈O (M+H)⁺: m/z=447.0; found 447.0.

### Step 3: 2-((5-amino-7-(3-cyanophenyl)-8-(pyrimidin-4-yl)-[1,2,4]triazolo[1,5-c]pyrimidin-2-yl)methoxy)nicotinonitrile

2-((5-amino-8-bromo-7-(3-cyanophenyl)-[1,2,4]triazolo[1,5-c]pyrimidin-2-yl)methoxy)nicotinonitrile (15 mg, 0.034 mmol), tetrakis (7.75 mg, 6.71 μmol), 4-(tributylstannyl)pyrimidine (18.57 mg, 0.050 mmol) were combined and 1,4-dioxane (1 ml) was added. The mixture was heated at 110° C. for 3 h, then cooled to room temperature, quenched and diluted with TFA (0.5 ml), filtered and purified by prep-LC-MS (pH=2, MeCN/water with TFA) to give the desired product as a TFA salt. LCMS calculated for C₂₃H₁₅N₁₀O (M+H)⁺: m/z=447.2; found 447.2.

### Example 43. 2-((5-amino-7-(3-cyanophenyl)-8-(1-methyl-6-oxo-1,6-dihydropyridin-3-yl)-[1,2,4]triazolo[1,5-c]pyrimidin-2-yl)methoxy)nicotinonitrile

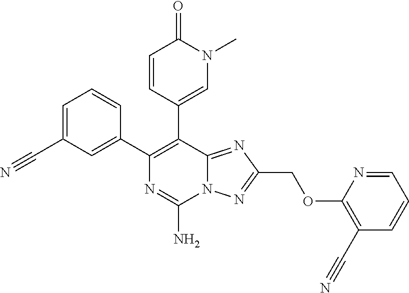 [SMILES: Cn1cc(-c2c(-c3cccc(C#N)c3)nc(N)n3nc(COc4ncccc4C#N)nc23)ccc1=O]

2-((5-(bis(4-methoxybenzyl)amino)-8-bromo-7-(3-cyanophenyl)-[1,2,4]triazolo[1,5-c]pyrimidin-2-yl)methoxy)nicotinonitrile (20 mg, 0.029 mmol), 1-methyl-5-(4,4,5,5-tetramethyl-1,3,2-dioxaborolan-2-yl)pyridin-2(1H)-one (13.68 mg, 0.058 mmol), sodium carbonate (6.17 mg, 0.058 mmol), Xphos-G2 (2.289 mg, 2.91 μmol) were combined. 1,4-dioxane (1 ml) and water (0.100 ml) were added and the mixture was heated to 100° C. and stirred for 3 h, then evaporated. To the residue was added TFA (1 ml) and the mixture was heated at 120° C. for 20 min. After cooled to room temperature, the mixture was diluted with acetonitrile, filtered and purified by prep-LC-MS (pH=2, MeCN/water with TFA) to give the desired product as a TFA salt. LCMS calculated for C₂₅H₁₈N₉O₂ (M+H)⁺: m/z=476.2; found 476.2.

### Example 44. 2-((5-amino-7-(3-cyanophenyl)-8-(1-methyl-6-oxo-1,6-dihydropyridazin-3-yl)-[1,2,4]triazolo[1,5-c]pyrimidin-2-yl)methoxy)nicotinonitrile

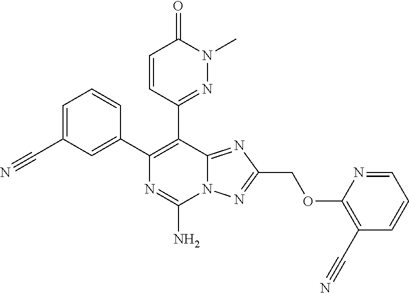 [SMILES: Cn1nc(-c2c(-c3cccc(C#N)c3)nc(N)n3nc(COc4ncccc4C#N)nc23)ccc1=O]

This compound was prepared using similar procedures as described in Example 43 using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₄H₁₇N₁₀O₂ (M+H)⁺: m/z=477.2; found 477.2.

### Example 45. 2-((5-amino-7-(3-cyanophenyl)-8-(3-methylpyridin-4-yl)-[1,2,4]triazolo[1,5-c]pyrimidin-2-yl)methoxy)nicotinonitrile

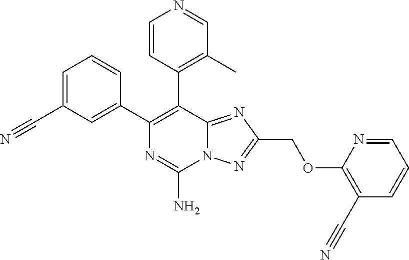 [SMILES: Cc1cnccc1-c1c(-c2cccc(C#N)c2)nc(N)n2nc(COc3ncccc3C#N)nc12]

This compound is prepared using similar procedures as described in Example 43, using 3-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material is purified by prep-LC-MS (pH=2, MeCN/water with TFA) to give the desired product as a TFA salt. LCMS calculated for C₂₅H₁₈N₉O (M+H)⁺: m/z=460.2.

### Example 46. 2-((5-amino-7-(3-cyanophenyl)-8-(2,6-dimethylpyridin-4-yl)-[1,2,4]triazolo[1,5-c]pyrimidin-2-yl)methoxy)nicotinonitrile

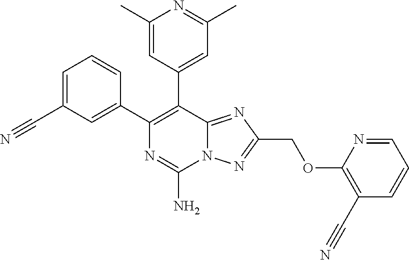 [SMILES: Cc1cc(-c2c(-c3cccc(C#N)c3)nc(N)n3nc(COc4ncccc4C#N)nc23)cc(C)n1]

This compound was prepared using similar procedures as described in Example 43 using 2,6-dimethyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₆H₂₀N₉O (M+H)⁺: m/z=474.2; found 474.2.

### Example 47. 2-((5-amino-7-(3-cyanophenyl)-8-(2-methoxy-6-methylpyridin-4-yl)-[1,2,4]triazolo[1,5-c]pyrimidin-2-yl)methoxy)nicotinonitrile

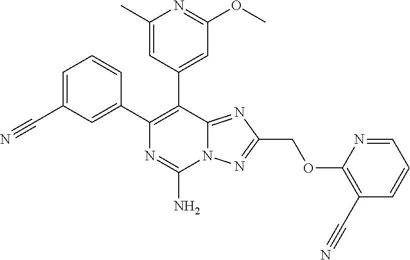 [SMILES: COc1cc(-c2c(-c3cccc(C#N)c3)nc(N)n3nc(COc4ncccc4C#N)nc23)cc(C)n1]

This compound was prepared using similar procedures as described in Example 43 using 2-methoxy-6-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to give the desired product as a TFA salt. LCMS calculated for C₂₆H₂₀N₉O₂ (M+H)⁺: m/z=490.2; found 490.2.

### Example 48. 3-(5-amino-2-((1-(pyridin-2-yl)ethyl)amino)-8-(pyrimidin-4-yl)-[1,2,4]triazolo[1,5-c]pyrimidin-7-yl)benzonitrile

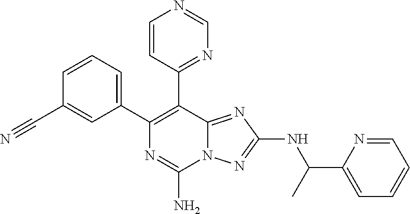 [SMILES: CC(Nc1nc2c(-c3ccncn3)c(-c3cccc(C#N)c3)nc(N)n2n1)c1ccccn1]

### Step 1: 3-(2-Amino-5-(bis(4-methoxybenzyl)amino)-8-bromo-[1,2,4]triazolo[1,5-c]pyrimidin-7-yl)benzonitrile

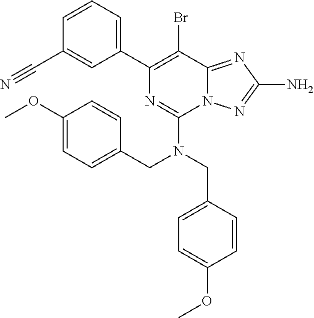 [SMILES: COc1ccc(CN(Cc2ccc(OC)cc2)c2nc(-c3cccc(C#N)c3)c(Br)c3nc(N)nn23)cc1]

To a solution of 3-(2-amino-5-(bis(4-methoxybenzyl)amino)-[1,2,4]triazolo[1,5-c]pyrimidin-7-yl)benzonitrile (Example 15, Step 3, 330 mg, 0.66 mmol) in DMF (1.4 ml) is slowly added NBS (120 mg, 0.66 mmol) at 0° C. The reaction mixture is then stirred at r.t. for 30 min before water (10 ml) is added. The resulting solid is collected by filtration, and dried to obtain the desired product. LC-MS calculated for C₂₈H₂₅BrN₇O₂ (M+H)⁺: m/z=570.1.

### Step 2: 3-(2-Amino-5-(bis(4-methoxybenzyl)amino)-8-(pyrimidin-4-yl)-[1,2,4]triazolo[1,5-c]pyrimidin-7-yl)benzonitrile

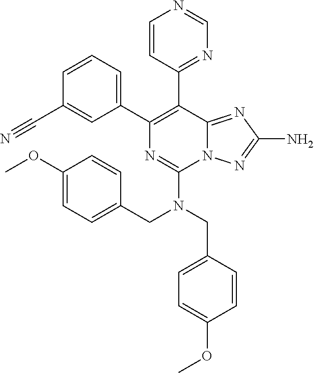 [SMILES: COc1ccc(CN(Cc2ccc(OC)cc2)c2nc(-c3cccc(C#N)c3)c(-c3ccncn3)c3nc(N)nn23)cc1]

A mixture of 3-(2-amino-5-(bis(4-methoxybenzyl)amino)-8-bromo-[1,2,4]triazolo[1,5-c]pyrimidin-7-yl)benzonitrile (350 mg, 0.61 mmol), 4-(tributylstannyl)pyrimidine (0.21 ml, 0.67 mmol), tetrakis(triphenylphosphine)palladium(0) (70 mg, 0.060 mmol), copper(I) iodide (23 mg, 0.12 mmol) and cesium fluoride (180 mg, 1.2 mmol) in dioxane (4.7 ml) is heated and stirred at 140° C. for 30 min in a microwave reactor. The reaction mixture is then cooled to room temperature, filtered through a Celite plug (washed with DCM), and concentrated. The resulting material is purified by silica gel column chromatography eluting with 0-20% MeOH/DCM to give the desired product. LC-MS calculated for C₃₂H₂₈N₉O₂ (M+H)⁺: m/z=570.2.

### Step 3: 3-(5-(bis(4-methoxybenzyl)amino)-2-bromo-8-(pyrimidin-4-yl)-[1,2,4]triazolo[1,5-c]pyrimidin-7-yl)benzonitrile

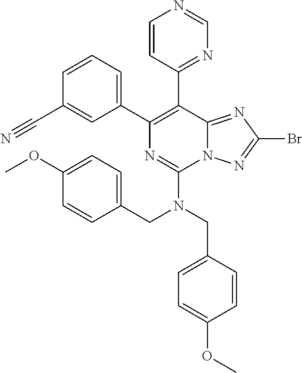 [SMILES: COc1ccc(CN(Cc2ccc(OC)cc2)c2nc(-c3cccc(C#N)c3)c(-c3ccncn3)c3nc(Br)nn23)cc1]

To a solution of copper(II) bromide (91 mg, 0.407 mmol) and tert-butyl nitrite (0.054 ml, 0.407 mmol) in acetonitrile (3 ml) under nitrogen at 50° C. is added dropwise 3-(2-amino-5-(bis(4-methoxybenzyl)amino)-8-(pyrimidin-4-yl)-[1,2,4]triazolo[1,5-c]pyrimidin-7-yl)benzonitrile (100 mg, 0.203 mmol) in acetonitrile (3 ml). The mixture is stirred at 50° C. for 2 hours. After cooling to room temperature, 1N aqueous NH₄OH solution (20 ml) is added and the mixture is extracted three times with CH₂Cl₂ (20 ml). The combined organic layers are dried over sodium sulfate, filtered, and the solvent is evaporated under reduced pressure. The residue is purified by silica gel column chromatography eluting with 50-100% ethyl acetate/hexane to give the desired product. LC-MS calculated for C₃₂H₂₆BrN₈O₂ (M+H)⁺: m/z=633.2.

### Step 4: 3-(5-amino-2-((1-(pyridin-2-yl)ethyl)amino)-8-(pyrimidin-4-yl)-[1,2,4]triazolo[1,5-c]pyrimidin-7-yl)benzonitrile

3-(5-(bis(4-methoxybenzyl)amino)-2-bromo-8-(pyrimidin-4-yl)-[1,2,4]triazolo[1,5-c]pyrimidin-7-yl)benzonitrile (750 mg, 1.184 mmol), 1-(pyridin-2-yl)ethan-1-amine (289 mg, 2.368 mmol), sodium tert-butoxide (228 mg, 2.368 mmol) and (t-Bu)PhCPhos Pd G3 (92 mg, 0.118 mmol) are combined and 1,4-dioxane (10 ml) is added. The mixture is heated at 60° C. for 3 h, and then cooled to room temperature and evaporated. To the residue is added TFA (5 ml) and the mixture is heated at 120° C. for 20 min. After cooling to room temperature, the mixture is diluted with acetonitrile, filtered, and purified by prep-LC-MS (pH=2, MeCN/water with TFA) to afford the desired product as a TFA salt. LCMS calculated for C₂₃H₉N₁₀ (M+H)⁺: m/z=435.2.

### Example 49. 3-(5-amino-2-((2-(pyridin-2-yl)propan-2-yl)amino)-8-(pyrimidin-4-yl)-[1,2,4]triazolo[1,5-c]pyrimidin-7-yl)benzonitrile

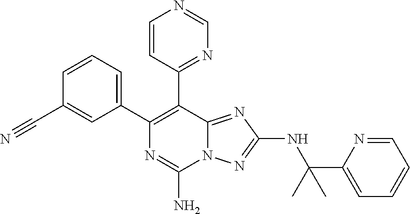 [SMILES: CC(C)(Nc1nc2c(-c3ccncn3)c(-c3cccc(C#N)c3)nc(N)n2n1)c1ccccn1]

This compound is prepared using similar procedures as described in Example 48 using 2-(pyridin-2-yl)propan-2-amine in place of 1-(pyridin-2-yl)ethan-1-amine. The final material is purified by prep-LC-MS (pH=2, MeCN/water with TFA) to give the desired product as a TFA salt. LCMS calculated for C₂₄H₂₁N₁₀ (M+H)⁺: m/z=449.2.

### Example 50. 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile

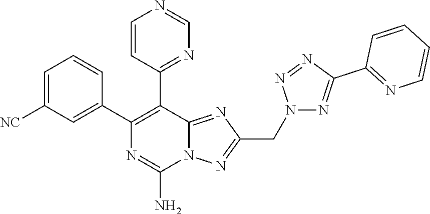 [SMILES: N#Cc1cccc(-c2nc(N)n3nc(Cn4nnc(-c5ccccn5)n4)nc3c2-c2ccncn2)c1]

This compound was prepared using similar procedures as described for Example 1, with 2-(1H-tetrazol-5-yl)pyridine replacing 3-(1H-tetrazol-5-yl)aniline. The product was purified by preparative LC-MS (pH 2, acetonitrile/water with TFA) to afford the product as a TFA salt. LCMS calculated for C₂₃H₁₆N₁₃ (M+H)⁺: 474.2; found 474.2

### Example 51. 3-(5-amino-2-((5-(pyrimidin-2-yl)-1H-tetrazol-1-yl)methyl)-8-(pyrimidin-4-yl)-[1,2,4]triazolo[1,5-c]pyrimidin-7-yl)benzonitrile

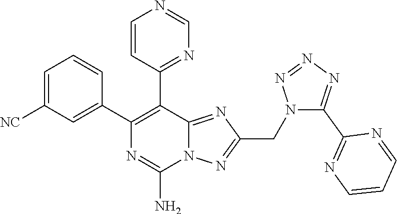 [SMILES: N#Cc1cccc(-c2nc(N)n3nc(Cn4nnnc4-c4ncccn4)nc3c2-c2ccncn2)c1]

This compound was prepared using similar procedures as described for Example 1, with 2-(1H-tetrazol-5-yl)pyrimidine replacing 3-(1H-tetrazol-5-yl)aniline. The product was purified by preparative LC-MS (pH 2, acetonitrile/water with TFA) to afford the product as a TFA salt. LCMS calculated for C₂₂H₁₅N₁₄ (M+H)⁺: 475.2; found 475.2

### Example 52. 3-(5-amino-8-(pyrimidin-4-yl)-2-((5-(pyrimidin-4-yl)-1H-tetrazol-1-yl)methyl)-[1,2,4]triazolo[1,5-c]pyrimidin-7-yl)benzonitrile

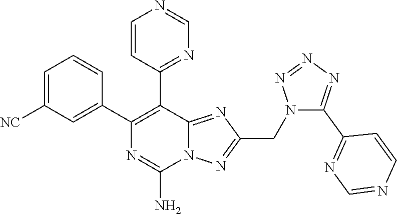 [SMILES: N#Cc1cccc(-c2nc(N)n3nc(Cn4nnnc4-c4ccncn4)nc3c2-c2ccncn2)c1]

This compound was prepared using similar procedures as described for Example 1, with 4-(1H-tetrazol-5-yl)pyrimidine replacing 3-(1H-tetrazol-5-yl)aniline. The product was purified by preparative LC-MS (pH 2, acetonitrile/water with TFA) to afford the product as a TFA salt. LCMS calculated for C₂₂H₁₅N₁₄ (M+H)⁺: 475.2; found 475.2

### Example 53. 3-(5-amino-2-((5-(pyridin-3-yl)-1H-tetrazol-1-yl)methyl)-8-(pyrimidin-4-yl)-[1,2,4]triazolo[1,5-c]pyrimidin-7-yl)benzonitrile

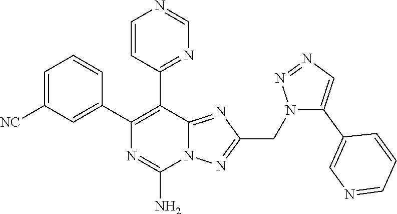 [SMILES: N#Cc1cccc(-c2nc(N)n3nc(Cn4nncc4-c4cccnc4)nc3c2-c2ccncn2)c1]

This compound was prepared using similar procedures as described for Example 1, with 3-(1H-tetrazol-5-yl)pyridine replacing 3-(1H-tetrazol-5-yl)aniline. The product was purified by preparative LC-MS (pH 2, acetonitrile/water with TFA) to afford the product as a TFA salt. LCMS calculated for C₂₃H₁₆N₁₃ (M+H)⁺: 474.2; found 474.2

### Example 54. 3-(5-amino-2-((5-(pyridin-4-yl)-1H-tetrazol-1-yl)methyl)-8-(pyrimidin-4-yl)-[1,2,4]triazolo[1,5-c]pyrimidin-7-yl)benzonitrile

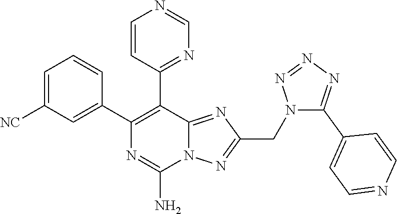 [SMILES: N#Cc1cccc(-c2nc(N)n3nc(Cn4nnnc4-c4ccncc4)nc3c2-c2ccncn2)c1]

This compound was prepared using similar procedures as described for Example 1, with 4-(1H-tetrazol-5-yl)pyridine replacing 3-(1H-tetrazol-5-yl)aniline. The product was purified by preparative LC-MS (pH 2, acetonitrile/water with TFA) to afford the product as a TFA salt. LCMS calculated for C₂₃H₁₆N₁₃ (M+H)⁺: 474.2; found 474.2

### Example 55. 3-(5-amino-8-(1-methyl-6-oxo-1,6-dihydropyridin-3-yl)-2-((6-methylpyridin-2-yl)methoxy)-[1,2,4]triazolo[1,5-c]pyrimidin-7-yl)benzonitrile

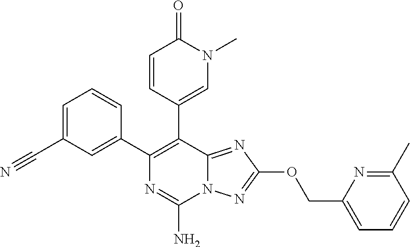 [SMILES: Cc1cccc(COc2nc3c(-c4ccc(=O)n(C)c4)c(-c4cccc(C#N)c4)nc(N)n3n2)n1]

### Step 1: 3-(5-(bis(4-methoxybenzyl)amino)-8-bromo-2-((6-methylpyridin-2-yl)methoxy)-[1,2,4]triazolo[1,5-c]pyrimidin-7-yl)benzonitrile

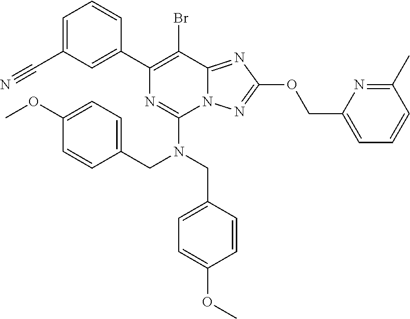

This compound was prepared using similar procedures as described in Example 28, using (6-methylpyridin-2-yl)methanol in place of (3-fluoropyridin-2-yl)methanol in step 1. The product was purified by column chromatography (10 to 60% AcOEt in hexane) to give the desired product. LCMS calculated for C₃₅H₃₁BrN₇O₃ (M+H)⁺: m/z=676.2; found 676.2.

### Step 2: 3-(5-amino-8-(I-methyl-6-oxo-1,6-dihydropyridin-3-yl)-2-((6-methylpyridin-2-yl)methoxy)-[1,2,4]triazolo[1,5-c]pyrimidin-7-yl)benzonitrile

3-(5-(bis(4-methoxybenzyl)amino)-8-bromo-2-((6-methylpyridin-2-yl)methoxy)-[1,2,4]triazolo[1,5-c]pyrimidin-7-yl)benzonitrile (20 mg, 0.030 mmol), 1-methyl-5-(4,4,5,5-tetramethyl-1,3,2-dioxaborolan-2-yl)pyridin-2(1H)-one (6.95 mg, 0.030 mmol), sodium carbonate (6.27 mg, 0.059 mmol), Xphos-G2 (2.326 mg, 2.96 μmol) were combined. To the mixture was added 1,4-dioxane (1 ml) and water (0.100 ml). The mixture was heated to 100° C. and stirred for 3 h, and then evaporated. To the residue was added TFA (1 ml) and the mixture was heated at 120° C. for 20 min. Then diluted with acetonitrile, filtered and purified by prep-LC-MS (pH=2, MeCN/water with TFA) to give products as a TFA salt. LCMS calculated for C₂₅H₂₁N802 (M+H)⁺: m/z=465.2; found 465.2.

### Example 56. 3-(5-amino-8-(1-methyl-6-oxo-1,6-dihydropyridazin-3-yl)-2-((6-methylpyridin-2-yl)methoxy)-[1,2,4]triazolo[1,5-c]pyrimidin-7-yl)benzonitrile

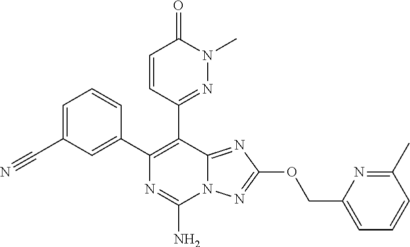 [SMILES: Cc1cccc(COc2nc3c(-c4ccc(=O)n(C)n4)c(-c4cccc(C#N)c4)nc(N)n3n2)n1]

This compound is prepared using similar procedures as described in Example 55, using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one in step 2. The final material is purified by prep-LC-MS (pH=2, MeCN/water with TFA) to afford the desired product as a TFA salt. LCMS calculated for C₂₄H2N₉O₂ (M+H)⁺: m/z=466.2.

### Example 57. 3-(5-amino-2-((6-methylpyridin-2-yl)methoxy)-8-(3-methylpyridin-4-yl)-[1,2,4]triazolo[1,5-c]pyrimidin-7-yl)benzonitrile

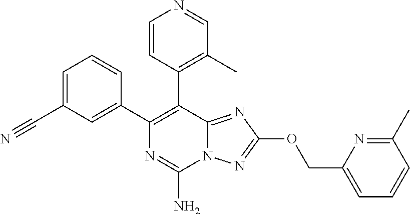 [SMILES: Cc1cccc(COc2nc3c(-c4ccncc4C)c(-c4cccc(C#N)c4)nc(N)n3n2)n1]

This compound was prepared using similar procedures as described in Example 55, using 3-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one in step 2. The final material was purified by prep-LC-MS (pH=2, MeCN/water with TFA) to the desired product as a TFA salt. LCMS calculated for C₂₅H₂₁N80 (M+H)⁺: m/z=449.2; found 449.2.

### Example 58. 3-(5-amino-8-(2,6-dimethylpyridin-4-yl)-2-((6-methylpyridin-2-yl)methoxy)-[1,2,4]triazolo[1,5-c]pyrimidin-7-yl)benzonitrile

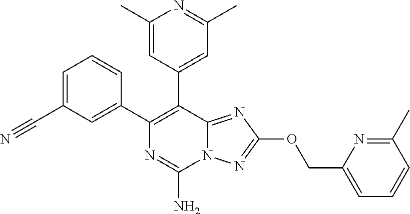 [SMILES: Cc1cc(-c2c(-c3cccc(C#N)c3)nc(N)n3nc(OCc4cccc(C)n4)nc23)cc(C)n1]

This compound was prepared using similar procedures as described in Example 55, using 2,6-dimethyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one in step 2. The final material was purified by prep-LC-MS (pH=2, MeCN/water with TFA) to the desired product as a TFA salt. LCMS calculated for C₂₆H₂₃N80 (M+H)⁺: m/z=463.2; found 463.2.

### Example 59. 3-(5-amino-8-(2-methoxy-6-methylpyridin-4-yl)-2-((6-methylpyridin-2-yl)methoxy)-[1,2,4]triazolo[1,5-c]pyrimidin-7-yl)benzonitrile

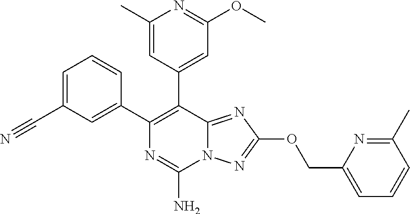 [SMILES: COc1cc(-c2c(-c3cccc(C#N)c3)nc(N)n3nc(OCc4cccc(C)n4)nc23)cc(C)n1]

This compound was prepared using similar procedures as described in Example 55, using 2-methoxy-6-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one in step 2. The final material was purified by prep-LC-MS (pH=2, MeCN/water with TFA) to the desired product as a TFA salt. LCMS calculated for C₂₆H₂₃N₈O₂ (M+H)⁺: m/z=479.2; found 479.2.

### Example 60. 3-(5-amino-2-((3,6-dimethylpyridin-2-yl)methoxy)-8-(1-methyl-6-oxo-1,6-dihydropyridin-3-yl)-[1,2,4]triazolo[1,5-c]pyrimidin-7-yl)benzonitrile

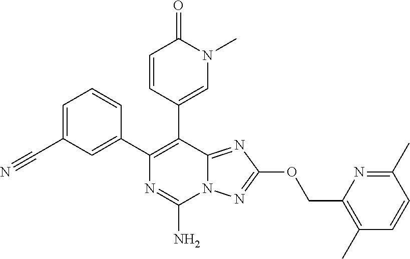 [SMILES: Cc1ccc(C)c(COc2nc3c(-c4ccc(=O)n(C)c4)c(-c4cccc(C#N)c4)nc(N)n3n2)n1]

### Step 1: 3-(5-(bis(4-methoxybenzyl)amino)-8-bromo-2-((3,6-dimethylpyridin-2-yl)methoxy)-[1,2,4]triazolo[1,5-c]pyrimidin-7-yl)benzonitrile

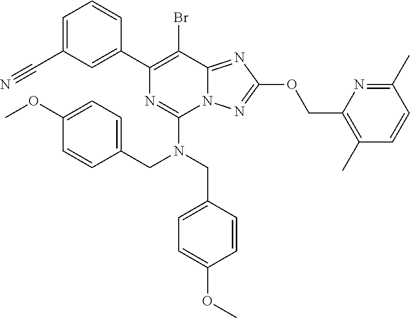 [SMILES: COc1ccc(CN(Cc2ccc(OC)cc2)c2nc(-c3cccc(C#N)c3)c(Br)c3nc(OCc4nc(C)ccc4C)nn23)cc1]

This compound is prepared using similar procedures as described in Example 28, using (3,6-dimethylpyridin-2-yl)methanol in place of (3-fluoropyridin-2-yl)methanol in step 1. The product is purified by column chromatography (10 to 60% AcOEt in hexane) to give the desired product. LCMS calculated for C₃₆H₃₃BrN₇O₃ (M+H)⁺: m/z=690.2.

### Step 2: 3-(5-amino-2-((3,6-dimethylpyridin-2-yl)methoxy)-8-(1-methyl-6-oxo-1,6-dihydropyridin-3-yl)-[1,2,4]triazolo[1,5-c]pyrimidin-7-yl)benzonitrile

3-(5-(bis(4-methoxybenzyl)amino)-8-bromo-2-((6-methylpyridin-2-yl)methoxy)-[1,2,4]triazolo[1,5-c]pyrimidin-7-yl)benzonitrile (20 mg, 0.030 mmol), 1-methyl-5-(4,4,5,5-tetramethyl-1,3,2-dioxaborolan-2-yl)pyridin-2(1H)-one (6.95 mg, 0.030 mmol), sodium carbonate (6.27 mg, 0.059 mmol), Xphos-G2 (2.326 mg, 2.96 μmol) are combined. To the mixture is added 1,4-dioxane (1 ml) and water (0.100 ml). The mixture is heated to 100° C. and stirred for 3 h, and then evaporated. To the residue is added TFA (1 ml) and the mixture is heated at 120° C. for 20 min. The mixture is then diluted with acetonitrile, filtered, and purified by prep-LC-MS (pH=2, MeCN/water with TFA) to give products as a TFA salt. LCMS calculated for C₂₆H₂₃N₈O₂ (M+H)⁺: m/z=479.2.

### Example 61. 3-(5-amino-2-((3,6-dimethylpyridin-2-yl)methoxy)-8-(1-methyl-6-oxo-1,6-dihydropyridazin-3-yl)-[1,2,4]triazolo[1,5-c]pyrimidin-7-yl)benzonitrile

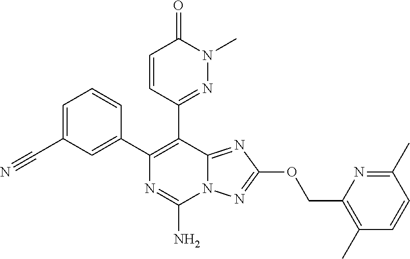 [SMILES: Cc1ccc(C)c(COc2nc3c(-c4ccc(=O)n(C)n4)c(-c4cccc(C#N)c4)nc(N)n3n2)n1]

This compound is prepared using similar procedures as described in Example 60, using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one in step 2. The final material is purified by prep-LC-MS (pH=2, MeCN/water with TFA) to afford the desired product as a TFA salt. LCMS calculated for C₂₅H₂₂N₉O₂ (M+H)⁺: m/z=480.2.

### Example 62. 3-(5-amino-2-((3,6-dimethylpyridin-2-yl)methoxy)-8-(3-methylpyridin-4-yl)-[1,2,4]triazolo[1,5-c]pyrimidin-7-yl)benzonitrile

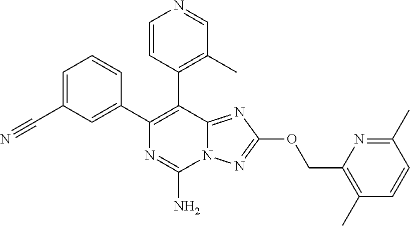 [SMILES: Cc1ccc(C)c(COc2nc3c(-c4ccncc4C)c(-c4cccc(C#N)c4)nc(N)n3n2)n1]

This compound is prepared using similar procedures as described in Example 60, using 3-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one in step 2. The final material is purified by prep-LC-MS (pH=2, MeCN/water with TFA) to afford the desired product as a TFA salt. LCMS calculated for C₂₆H₂₃N₈O (M+H)⁺: m/z=463.2.

### Example 63. 3-(5-amino-2-((3,6-dimethylpyridin-2-yl)methoxy)-8-(2,6-dimethylpyridin-4-yl)-[1,2,4]triazolo[1,5-c]pyrimidin-7-yl)benzonitrile

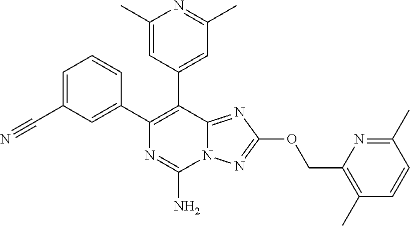 [SMILES: Cc1cc(-c2c(-c3cccc(C#N)c3)nc(N)n3nc(OCc4nc(C)ccc4C)nc23)cc(C)n1]

This compound is prepared using similar procedures as described in Example 60, using 2,6-dimethyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one in step 2. The final material is purified by prep-LC-MS (pH=2, MeCN/water with TFA) to afford the desired product as a TFA salt. LCMS calculated for C₂₇H₂₅N₈O (M+H)⁺: m/z=477.2.

### Example 64. 3-(5-amino-2-((3,6-dimethylpyridin-2-yl)methoxy)-8-(2-methoxy-6-methylpyridin-4-yl)-[1,2,4]triazolo[1,5-c]pyrimidin-7-yl)benzonitrile

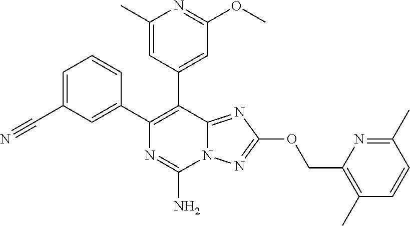 [SMILES: COc1cc(-c2c(-c3cccc(C#N)c3)nc(N)n3nc(OCc4nc(C)ccc4C)nc23)cc(C)n1]

This compound is prepared using similar procedures as described in Example 60, using 2-methoxy-6-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one in step 2. The final material is purified by prep-LC-MS (pH=2, MeCN/water with TFA) to afford the desired product as a TFA salt. LCMS calculated for C₂₇H₂₅N₈O₂ (M+H)⁺: m/z=493.2.

### Example 65. 3-(5-amino-2-(((6-methylpyridin-2-yl)methyl)amino)-8-(pyrimidin-4-yl)-[1,2,4]triazolo[1,5-c]pyrimidin-7-yl)benzonitrile

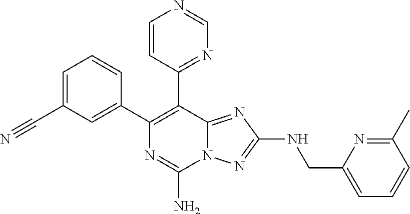 [SMILES: Cc1cccc(CNc2nc3c(-c4ccncn4)c(-c4cccc(C#N)c4)nc(N)n3n2)n1]

### Step 1: 3-(5-(bis(4-methoxybenzyl)amino)-8-bromo-2-(((6-methylpyridin-2-yl)methyl)amino)-[1,2,4]triazolo[1,5-c]pyrimidin-7-yl)benzonitrile

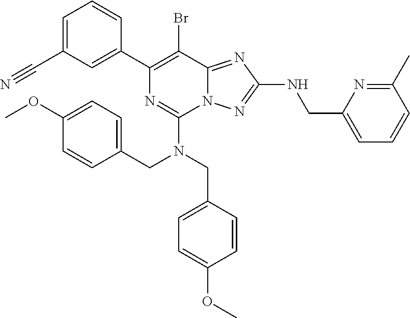 [SMILES: COc1ccc(CN(Cc2ccc(OC)cc2)c2nc(-c3cccc(C#N)c3)c(Br)c3nc(NCc4cccc(C)n4)nn23)cc1]

This compound was prepared using similar procedures as described in Example 15, using 6-methylpicolinaldehyde in place of 3-methylpicolinaldehyde in step 4. The product was purified by column chromatography (10 to 60% AcOEt in hexane) to give the desired product. LCMS calculated for C₃₅H₃₂BrN₈O₂ (M+H)⁺: m/z=675.2; found 675.2.

### Step 2: 3-(5-amino-2-(((6-methylpyridin-2-yl)methyl)amino)-8-(pyrimidin-4-yl)-[1,2,4]triazolo[1,5-c]pyrimidin-7-yl)benzonitrile

3-(5-(bis(4-methoxybenzyl)amino)-8-bromo-2-(((6-methylpyridin-2-yl)methyl)amino)-[1,2,4]triazolo[1,5-c]pyrimidin-7-yl)benzonitrile (20 mg, 0.030 mmol), tetrakis (6.84 mg, 5.92 μmol), 4-(tributylstannyl)pyrimidine (16.39 mg, 0.044 mmol) were combined. To the mixture was added 1,4-dioxane (1 ml). The mixture was heated at 110° C. for 3 h, evaporated. To the residue was added TFA (1 ml) and the mixture was heated at 120° C. for 20 min, and then diluted with acetonitrile, filtered and purified by prep-LC-MS (pH=2, MeCN/water with TFA) to give the product as a TFA salt. LCMS calculated for C₂₃H₁₉N₁₀ (M+H)⁺: m/z=435.2; found 435.2.

### Example 66. 3-(5-amino-8-(1-methyl-6-oxo-1,6-dihydropyridin-3-yl)-2-(((6-methylpyridin-2-yl)methyl)amino)-[1,2,4]triazolo[1,5-c]pyrimidin-7-yl)benzonitrile

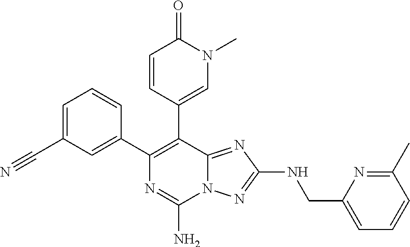 [SMILES: Cc1cccc(CNc2nc3c(-c4ccc(=O)n(C)c4)c(-c4cccc(C#N)c4)nc(N)n3n2)n1]

3-(5-(bis(4-methoxybenzyl)amino)-8-bromo-2-(((6-methylpyridin-2-yl)methyl)amino)-[1,2,4]triazolo[1,5-c]pyrimidin-7-yl)benzonitrile (20 mg, 0.030 mmol), 1-methyl-5-(4,4,5,5-tetramethyl-1,3,2-dioxaborolan-2-yl)pyridin-2(1H)-one (6.96 mg, 0.030 mmol), sodium carbonate (6.28 mg, 0.059 mmol), Xphos-G2 (2.329 mg, 2.96 μmol) are combined. To the mixture is added 1,4-dioxane (1 ml) and water (0.100 ml). The mixture is heated at 100° C. for 3 h, and then evaporated. To the residue is added TFA (1 ml) and the mixture is heated at 120° C. for 20 min, and then diluted with acetonitrile, filtered, and purified by prep-LC-MS (pH=2, MeCN/water with TFA) to give products as a TFA salt. LCMS calculated for C₂₅H₂₂N₉O (M+H)⁺: m/z=464.2.

### Example 67. 3-(5-amino-8-(1-methyl-6-oxo-1,6-dihydropyridazin-3-yl)-2-(((6-methylpyridin-2-yl)methyl)amino)-[1,2,4]triazolo[1,5-c]pyrimidin-7-yl)benzonitrile

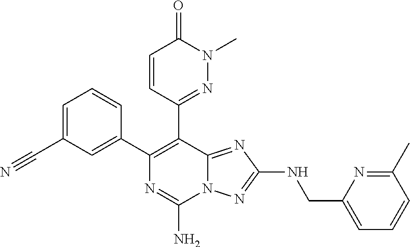 [SMILES: Cc1cccc(CNc2nc3c(-c4ccc(=O)n(C)n4)c(-c4cccc(C#N)c4)nc(N)n3n2)n1]

This compound was prepared using similar procedures as described in Example 66, using 2-methyl-6-(4,4,5,5-tetramethyl-1,3,2-dioxaborolan-2-yl)pyridazin-3(2H)-one in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to the desired product as a TFA salt. LCMS calculated for C₂₄H₂₁N₁₀O (M+H)⁺: m/z=465.2; found 465.2.

### Example 68. 3-(5-amino-2-(((6-methylpyridin-2-yl)methyl)amino)-8-(3-methylpyridin-4-yl)-[1,2,4]triazolo[1,5-c]pyrimidin-7-yl)benzonitrile

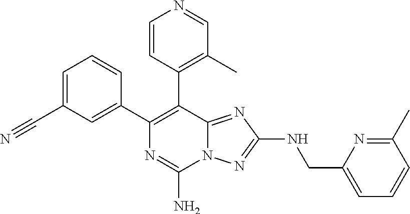 [SMILES: Cc1cccc(CNc2nc3c(-c4ccncc4C)c(-c4cccc(C#N)c4)nc(N)n3n2)n1]

This compound was prepared using similar procedures as described in Example 66, using 3-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one in. The final material was purified by prep-LC-MS (pH=2, MeCN/water with TFA) to the desired product as a TFA salt. LCMS calculated for C₂₅H₂₂N9 (M+H)⁺: m/z=448.2; found 448.2.

### Example 69. 3-(5-amino-8-(2,6-dimethylpyridin-4-yl)-2-(((6-methylpyridin-2-yl)methyl)amino)-[1,2,4]triazolo[1,5-c]pyrimidin-7-yl)benzonitrile

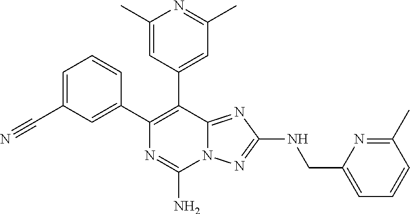 [SMILES: Cc1cc(-c2c(-c3cccc(C#N)c3)nc(N)n3nc(NCc4cccc(C)n4)nc23)cc(C)n1]

This compound was prepared using similar procedures as described in Example 66, using 2,6-dimethyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to the desired product as a TFA salt. LCMS calculated for C₂₆H₂₄N9 (M+H)⁺: m/z=462.2; found 462.2.

### Example 70. 3-(5-amino-8-(2-methoxy-6-methylpyridin-4-yl)-2-(((6-methylpyridin-2-yl)methyl)amino)-[1,2,4]triazolo[1,5-c]pyrimidin-7-yl)benzonitrile

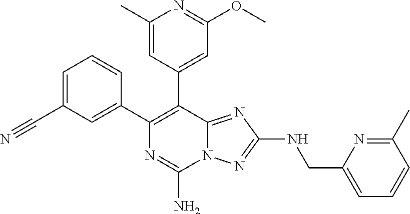 [SMILES: COc1cc(-c2c(-c3cccc(C#N)c3)nc(N)n3nc(NCc4cccc(C)n4)nc23)cc(C)n1]

This compound was prepared using similar procedures as described in Example 66, using 2-methoxy-6-methyl-4-(4,4,5,5-tetramethyl-1,3,2-dioxaborolan-2-yl)pyridine in place of 1-methyl-5-(4,4,5,5-tetramethyl-1,3,2-dioxaborolan-2-yl)pyridin-2(1H)-one. The final material was purified by prep-LC-MS (pH=2, MeCN/water with TFA) to the desired product as a TFA salt. LCMS calculated for C₂₆H₂₄N₉O (M+H)⁺: m/z=478.2; found 478.2.

### Example A. Adenosine A2A Receptor Cyclic AMP GS Assay

Stably transfected HEK-293 cells expressing the human adenosine A2A receptor (Perkin Elmer) are maintained in MEM culture medium with 10% FBS and 400 μg/ml Geneticin (Life Technologies). 18 to 24 hours prior to assay, geneticin is removed from culture. The cisbio cAMP-GS Dynamic kit utilizing the FRET (Fluorescence Resonance Energy Transfer) technology is used to measure cAMP accumulation in the cells. Compounds of the present disclosure at an appropriate concentration are mixed with 10000 cells/well in white 96 well half area plates (Perkin Elmer) for 30 min at room temperature (RT) gently shaking. Agonist, CGS21680 (R&D Technologies) at 4 nM is added to each well for 60 min at RT gently shaking. Detection reagents, d2-labeled cAMP (acceptor) and anti-cAMP cryptate (donor) are added to each well for 60 min at RT gently shaking. Plates are read on Pherastar (BMG Labtech), fluorescence ratio 665/620 is calculated and EC₅₀ determination is performed by fitting the curve of percent of control versus the log of the compound concentration using GraphPad Prism.

### Example B. Adenosine A2B Receptor Cyclic AMP GS Assay

Stably transfected HEK-293 cells expressing the human adenosine A2B receptor (Perkin Elmer) were maintained in MEM culture medium with 10% FBS and 100 μg/ml Geneticin (Life Technologies). 18 to 24 hours prior to assay, geneticin was removed from culture. The cisbio cAMP-GS Dynamic kit utilizing the FRET (Fluorescence Resonance Energy Transfer) technology was used to measure cAMP accumulation in the cells. Compounds of the present disclosure at an appropriate concentration were mixed with 10000 cells/well in white 96 well half area plates (Perkin Elmer) for 30 min at RT gently shaking. Agonist, NECA (R&D Technologies) at 12 nM was added to each well for 60 min at RT gently shaking. Detection reagents, d2-labeled cAMP (acceptor) and anti-cAMP cryptate (donor) were added to each well for 60 min at RT gently shaking. Plates were read on Pherastar (BMG Labtech), fluorescence ratio 665/620 was calculated and EC₅₀ determination was performed by fitting the curve of percent of control versus the log of the compound concentration using GraphPad Prism. The EC₅₀ data obtained via this method are shown in Table 1.

### Example C. A2A Tag-lite® HTRF Assay

Assays were conducted in black low volume 384-well polystyrene plates (Greiner 784076-25) in a final volume of 10 μL. Test compounds were first serially diluted in DMSO and 100 nl added to the plate wells before the addition of other reaction components. The final concentration of DMSO was 1%. Tag-lite® Adenosine A2A labeled cells (CisBio C1TT1A2A) were diluted 1:5 into Tag-lite buffer (CisBio LABMED) and spun 1200 g for 5 mins. The pellet was resuspended at a volume 10.4× the initial cell suspension volume in Tag-lite buffer, and Adenosine A2A Receptor Red antagonist fluorescent ligand (CisBio L0058RED) added at 12.5 nM final concentration. 10 ul of the cell and ligand mix was added to the assay wells and incubated at room temperature for 45 minutes before reading on a PHERAstar FS plate reader (BMG Labtech) with HTRF 337/620/665 optical module. Percent binding of the fluorescent ligand was calculated; where 100 nM of A2A antagonist control ZM 241385 (Tocris 1036) displaces the ligand 100% and 1% DMSO has 0% displacement. The % binding data versus the log of the inhibitor concentration was fitted to a one-site competitive binding model (GraphPad Prism version 7.02) where the ligand constant=12.5 nM and the ligand Kd=1.85 nM. The Kᵢ data obtained via this method are shown in Table 1.

### Example D. A2B Filter Binding Assay

Assays are conducted in deep well polypropylene plates (Greiner 786201) in a final volume of 550 μL. Test compounds are first serially diluted in DMSO and 5.5 ul is then added to the plate wells before the addition of other reaction components. The final concentration of DMSO is 3%. HEK293 cell membranes overexpressing the human adenosine receptor A2B (Perkin Elmer ES-113-M400UA) are diluted to 40 μg/ml in 50 mM HEPES pH 7.0, 5 mM MgCl₂, 1 mM EDTA (Assay buffer). [3H] 8-cyclopentyl-1,3-dipropylxanthine (Perkin Elmer NET974001MC) is diluted in assay buffer+22% DMSO to 24.2 nM, and then further diluted to 1 nM by addition to the diluted membranes. 545 μl of the membrane and ligand mix is added to the assay wells and incubated on a shaker at room temperature for 1 hour. The membrane mix is then filtered over a UniFilter GF/C filter plate (Perkin Elmer 6005174) pre-soaked in 50 mM HEPES pH 6.5, 5 mM MgCl₂, 1 mM EDTA 0.5% BSA and then washed with 5 ml ice cold 50 mM HEPES pH 6.5, 5 mM MgCl₂, 1 mM EDTA 0.2% BSA. 50 μl MicroScint™ cocktail (Perkin Elmer 6013621) is added and plates are read on a Topcount NXT FS (Perkin Elmer). Percent binding of the [3H] ligand is calculated, where 1000 nM of LUF 5834 (Tocris 4603) control displaces the ligand 100% and 3% DMSO has 0% displacement. The % binding data versus the log of the inhibitor concentration is fitted to a one-site competitive binding model (GraphPad Prism version 7.02) where the ligand constant=2 nM and the ligand Kd=13 nM.

### Example E. A1 and A3 SPA Binding Assays

Both assays are conducted in white 384-well polystyrene plates (Greiner 781075) in a final volume of 50 L. Inhibitors are first serially diluted in DMSO and 100 nL is added to the plate wells before the addition of other reaction components. The final concentration of DMSO is 2%.

Wheatgerm agglutinin-coated yttrium silicate SPA beads (Perkin Elmer RPNQ0023) and CHO-K1 cell membranes overexpressing each human adeonsine receptor are incubated in 50 mM HEPES pH 7.0, 5 mM MgCl₂, 1 mM EDTA (Assay buffer) on a rotary stirrer for 2 hours at 4° C. The beads are pelleted by centrifugation at 6000 g for one minute, and then the supernatant with unbound membrane is discarded. The beads are re-suspended to the original volume in assay buffer. Each radioligand is diluted in assay buffer+22% DMSO at 12.2× the final concentration, and then added to the SPA bead suspension. 50 μl of the SPA bead reaction mix is added to the assay wells and the plates shaken at 600 rpm for 1 hour at room temperature. The beads are then allowed to settle for 1 hour before reading on a Topcount NXT FS (Perkin Elmer). Percent binding of the radiolabeled ligand is calculated, where a control at >100× Ki displaces the ligand 100% and 2% DMSO has 0% displacement. The % binding data versus the log of the inhibitor concentration is fitted to a one-site competitive binding model (GraphPad Prism version 7.02). Assay conditions are provided in the table below.

| Assay | | |
|---|---|---|
| Component | A1 | A3 |
| SPA beads in | 3 mg/ml | 1.25 mg/ml |
| Hepes buffer | | |
| Membrane | 60 μg/ml | 20 μg/ml |
| | Perkin Elmer ES-010 | Perkin Elemer ES-012 |
| Radioligand | 1 nM [3H] DP-CPX | 0.1 nM [1251] MECA |
| | (Perkin Elmer NET974) | (Perkin Elmer NEX312) |
| | K_{D} = 1 nM | K_{D} = 0.8 nM |
| Control | 1 μM DPCPX | 0.1 μM IB-MECA |
| | (Tocris 0439) | (Tocris 1066) |

TABLE 1
The A_{2A}_Ki data (Example C) and A_{2B}_cAMP_EC₅₀ data
(Example B) are provided below.

| Ex. | A2A_Ki | A2B_cAMP_ |
|---|---|---|
| No. | (nM) | EC50 (nM) |
| 1 | † | † |
| 2 | † | † |
| 3 | † | †† |
| 4 | † | †† |
| 5 | † | †† |
| 6 | † | †† |
| 7 | † | †† |
| 8 | † | †† |
| 9 | † | †† |
| 10 | † | † |
| 11 | † | † |
| 12 | † | † |
| 13 | † | † |
| 14 | † | † |
| 15 | † | † |
| 16 | † | † |
| 17 | † | † |
| 18 | † | † |
| 19 | † | † |
| 20 | † | † |
| 21 | † | †† |
| 22 | † | †† |
| 23 | † | †† |
| 24 | † | †† |
| 25 | † | † |
| 26 | † | †† |
| 27 | † | † |
| 28 | † | † |
| 29 | † | † |
| 30 | † | † |
| 31 | NA | NA |
| 32 | † | †† |
| 33 | † | † |
| 34 | † | † |
| 35 | NA | NA |
| 36 | NA | NA |
| 37 | NA | NA |
| 38 | NA | NA |
| 39 | NA | NA |
| 40 | † | † |
| 41 | † | † |
| 42 | † | † |
| 43 | † | † |
| 44 | † | †† |
| 45 | NA | NA |
| 46 | † | ††† |
| 47 | † | †† |
| 48 | NA | NA |
| 49 | NA | NA |
| 50 | † | † |
| 51 | † | † |
| 52 | † | † |
| 53 | † | † |
| 54 | † | † |
| 55 | † | †† |
| 56 | NA | NA |
| 57 | † | †† |
| 58 | † | ††† |
| 59 | † | †† |
| 60 | NA | NA |
| 61 | NA | NA |
| 62 | NA | NA |
| 63 | NA | NA |
| 64 | NA | NA |
| 65 | † | †† |
| 66 | NA | NA |
| 67 | † | †† |
| 68 | † | † |
| 69 | † | †††† |
| 70 | † | ††† |

†indicates A_{2A}_Ki or A_{2B}_cAMP_EC₅₀ ≤10 nM,
††indicates A_{2A}_Ki or A_{2B}_cAMP_EC₅₀ >10 nM but ≤100 nM,
†††indicates A_{2A}_Ki or A_{2B}_cAMP_EC₅₀ >100 nM but ≤1 μM,
††††indicates A_{2A}_Ki or A_{2B}_cAMP_EC₅₀ is greater than 1 μM, and
NA indicates “not available”.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound selected from:
3-(5-amino-2-((5-(3-aminophenyl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(6-methylpyridin-2-yl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(6-methoxypyridin-2-yl)-1H-tetrazol-1-yl)methyl)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyridin-2-yl)-1H-tetrazol-1-yl)methyl)-[1,2,4]triazolo[1,5-c]pyrimidin-7-yl)benzonitrile;
3-(2-((5-(1H-pyrazol-1-yl)-1H-tetrazol-1-yl)methyl)-5-amino-8-(1-methyl-6-oxo-1,6-dihydropyridin-3-yl)-[1,2,4]triazolo[1,5-c]pyrimidin-7-yl)benzonitrile;
3-(5-amino-8-(1-methyl-6-oxo-1,6-dihydropyridin-3-yl)-2-((5-(thiazol-4-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyrimidin-2-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridin-3-yl)-2-((5-(pyrazin-2-yl)-1H-tetrazol-1-yl)methyl)-[1,2,4]triazolo[1,5-c]pyrimidin-7-yl)benzonitrile;
3-(5-amino-8-(1-methyl-6-oxo-1,6-dihydropyridazin-3-yl)-2-((5-(pyridin-2-yl)-1H-tetrazol-1-yl)methyl)-[1,2,4]triazolo[1,5-c]pyrimidin-7-yl)benzonitrile;
3-(5-amino-2-((5-(pyridin-2-yl)-1H-tetrazol-1-yl)methyl)-8-(pyridin-4-yl)-[1,2,4]triazolo[1,5-c]pyrimidin-7-yl)benzonitrile;
3-(5-amino-8-(2-(dimethylamino)pyridin-4-yl)-2-((5-(pyridin-2-yl)-1H-tetrazol-1-yl)methyl)-[1,2,4]triazolo[1,5-c]pyrimidin-7-yl)benzonitrile;
3-(5-amino-8-(2-aminopyridin-4-yl)-2-((5-(pyridin-2-yl)-1H-tetrazol-1-yl)methyl)-[1,2,4]triazolo[1,5-c]pyrimidin-7-yl)benzonitrile;
3-(5-amino-2-(2-fluoro-6-(pyridin-4-yl)benzyl)-8-(pyridin-4-yl)-[1,2,4]triazolo[1,5-c]pyrimidin-7-yl)benzonitrile;
3-(5-amino-8-(2-aminopyridin-4-yl)-2-(2-(2-aminopyridin-4-yl)-6-fluorobenzyl)-[1,2,4]triazolo[1,5-c]pyrimidin-7-yl)benzonitrile;
3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)benzonitrile;
3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridin-3-yl)-2-((3-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridazin-3-yl)-2-((3-methylpyridin-2-yl)methoxy)-[1,2,4]triazolo[1,5-c]pyrimidin-7-yl)benzonitrile;
3-(5-amino-2-((3-methylpyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((3-methylpyridin-2-yl)methoxy)-[1,2,4]triazolo[1,5-c]pyrimidin-7-yl)benzonitrile;
3-(5-amino-8-(1-ethyl-6-oxo-1,6-dihydropyridin-3-yl)-2-((3-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(1-ethyl-6-oxo-1,6-dihydropyridin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(1-methyl-6-oxo-1,6-dihydropyridazin-3-yl)-2-(((3-methylpyridin-2-yl)methyl)amino)-[1,2,4]triazolo[1,5-c]pyrimidin-7-yl)-2-fluorobenzonitrile;
3-(5-amino-8-(2-methoxy-6-methylpyridin-4-yl)-2-(((3-methylpyridin-2-yl)methyl)amino)-[1,2,4]triazolo[1,5-c]pyrimidin-7-yl)-2-fluorobenzonitrile;
3-(5-amino-8-(1-methyl-6-oxo-1,6-dihydropyridazin-3-yl)-2-(pyridin-2-ylmethyl)-[1,2,4]triazolo[1,5-c]pyrimidin-7-yl)benzonitrile;
3-(5-amino-2-((3-fluoropyridin-2-yl)methoxy)-8-(pyrimidin-4-yl)-[1,2,4]triazolo[1,5-c]pyrimidin-7-yl)benzonitrile;
3-(5-amino-2-((3-fluoropyridin-2-yl)methoxy)-8-(1-methyl-6-oxo-1,6-dihydropyridin-3-yl)-[1,2,4]triazolo[1,5-c]pyrimidin-7-yl)benzonitrile;
3-(5-amino-2-((3-fluoropyridin-2-yl)methoxy)-8-(1-methyl-6-oxo-1,6-dihydropyridazin-3-yl)-[1,2,4]triazolo[1,5-c]pyrimidin-7-yl)benzonitrile;
3-(5-amino-2-((3-fluoropyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((3-fluoropyridin-2-yl)methoxy)-[1,2,4]triazolo[1,5-c]pyrimidin-7-yl)benzonitrile;
3-(5-amino-2-((3-fluoropyridin-2-yl)methoxy)-8-(2-methoxy-6-methylpyridin-4-yl)-[1,2,4]triazolo[1,5-c]pyrimidin-7-yl)benzonitrile;
3-(5-amino-2-(pyridin-2-ylamino)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-(pyridin-2-ylamino)-[1,2,4]triazolo[1,5-c]pyrimidin-7-yl)benzonitrile;
3-(5-amino-8-(1-methyl-6-oxo-1,6-dihydropyridazin-3-yl)-2-(pyridin-2-ylamino)-[1,2,4]triazolo[1,5-c]pyrimidin-7-yl)benzonitrile;
3-(5-amino-8-(3-methylpyridin-4-yl)-2-(pyridin-2-ylamino)-[1,2,4]triazolo[1,5-c]pyrimidin-7-yl)benzonitrile;
3-(5-amino-8-(2,6-dimethylpyridin-4-yl)-2-(pyridin-2-ylamino)-[1,2,4]triazolo[1,5-c]pyrimidin-7-yl)benzonitrile;
3-(5-amino-8-(2-methoxy-6-methylpyridin-4-yl)-2-(pyridin-2-ylamino)-[1,2,4]triazolo[1,5-c]pyrimidin-7-yl)benzonitrile;
3-(5-amino-2-((6-methylpyridin-2-yl)amino)-8-(pyrimidin-4-yl)-[1,2,4]triazolo[1,5-c]pyrimidin-7-yl)benzonitrile;
3-(5-amino-2-((pyridin-2-yloxy)methyl)-8-(pyrimidin-4-yl)-[1,2,4]triazolo[1,5-c]pyrimidin-7-yl)benzonitrile;
2-((5-amino-7-(3-cyanophenyl)-8-(pyrimidin-4-yl)-[1,2,4]triazolo[1,5-c]pyrimidin-2-yl)methoxy)nicotinonitrile;
2-((5-amino-7-(3-cyanophenyl)-8-(1-methyl-6-oxo-1,6-dihydropyridin-3-yl)-[1,2,4]triazolo[1,5-c]pyrimidin-2-yl)methoxy)nicotinonitrile;
2-((5-amino-7-(3-cyanophenyl)-8-(1-methyl-6-oxo-1,6-dihydropyridazin-3-yl)-[1,2,4]triazolo[1,5-c]pyrimidin-2-yl)methoxy)nicotinonitrile;
2-((5-amino-7-(3-cyanophenyl)-8-(3-methylpyridin-4-yl)-[1,2,4]triazolo[1,5-c]pyrimidin-2-yl)methoxy)nicotinonitrile;
2-((5-amino-7-(3-cyanophenyl)-8-(2,6-dimethylpyridin-4-yl)-[1,2,4]triazolo[1,5-c]pyrimidin-2-yl)methoxy)nicotinonitrile;
2-((5-amino-7-(3-cyanophenyl)-8-(2-methoxy-6-methylpyridin-4-yl)-[1,2,4]triazolo[1,5-c]pyrimidin-2-yl)methoxy)nicotinonitrile;
3-(5-amino-2-((1-(pyridin-2-yl)ethyl)amino)-8-(pyrimidin-4-yl)-[1,2,4]triazolo[1,5-c]pyrimidin-7-yl)benzonitrile;
3-(5-amino-2-((2-(pyridin-2-yl)propan-2-yl)amino)-8-(pyrimidin-4-yl)-[1,2,4]triazolo[1,5-c]pyrimidin-7-yl)benzonitrile;
3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile;
3-(5-amino-2-((5-(pyrimidin-2-yl)-1H-tetrazol-1-yl)methyl)-8-(pyrimidin-4-yl)-[1,2,4]triazolo[1,5-c]pyrimidin-7-yl)benzonitrile;
3-(5-amino-8-(pyrimidin-4-yl)-2-((5-(pyrimidin-4-yl)-1H-tetrazol-1-yl)methyl)-[1,2,4]triazolo[1,5-c]pyrimidin-7-yl)benzonitrile;
3-(5-amino-2-((5-(pyridin-3-yl)-1H-tetrazol-1-yl)methyl)-8-(pyrimidin-4-yl)-[1,2,4]triazolo[1,5-c]pyrimidin-7-yl)benzonitrile;
3-(5-amino-2-((5-(pyridin-4-yl)-1H-tetrazol-1-yl)methyl)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-((6-methylpyridin-2-yl)methoxy)-[1,2,4]triazolo[1,5-c]pyrimidin-7-yl)benzonitrile;
3-(5-amino-8-(1-methyl-6-oxo-1,6-dihydropyridazin-3-yl)-2-((6-methylpyridin-2-yl)methoxy)-[1,2,4]triazolo[1,5-c]pyrimidin-7-yl)benzonitrile;
3-(5-amino-2-((6-methylpyridin-2-yl)methoxy)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-((6-methylpyridin-2-yl)methoxy)-[1,2,4]triazolo[1,5-c]pyrimidin-7-yl)benzonitrile;
3-(5-amino-8-(2-methoxy-6-methylpyridin-4-yl)-2-((6-methylpyridin-2-yl)methoxy)-[1,2,4]triazolo[1,5-c]pyrimidin-7-yl)benzonitrile;
3-(5-amino-2-((3,6-dimethylpyridin-2-yl)methoxy)-8-(1-methyl-6-oxo-1,6-dihydropyridin-3-yl)-[1,2,4]triazolo[1,5-c]pyrimidin-7-yl)benzonitrile;
3-(5-amino-2-((3,6-dimethylpyridin-2-yl)methoxy)-8-(1-methyl-6-oxo-1,6-dihydropyridazin-3-yl)-[1,2,4]triazolo[1,5-c]pyrimidin-7-yl)benzonitrile;
3-(5-amino-2-((3,6-dimethylpyridin-2-yl)methoxy)-8-(3-methylpyridin-4-yl)-[1,2,4]triazolo[1,5-c]pyrimidin-7-yl)benzonitrile;
3-(5-amino-2-((3,6-dimethylpyridin-2-yl)methoxy)-8-(2,6-dimethylpyridin-4-yl)-[1,2,4]triazolo[1,5-c]pyrimidin-7-yl)benzonitrile;
3-(5-amino-2-((3,6-dimethylpyridin-2-yl)methoxy)-8-(2-methoxy-6-methylpyridin-4-yl)-[1,2,4]triazolo[1,5-c]pyrimidin-7-yl)benzonitrile;
3-(5-amino-2-(((6-methylpyridin-2-yl)methyl)amino)-8-(pyrimidin-4-yl)-[1,2,4]triazolo[1,5-c]pyrimidin-7-yl)benzonitrile;
3-(5-amino-8-(1-methyl-6-oxo-1,6-dihydropyridin-3-yl)-2-(((6-methylpyridin-2-yl)methyl)amino)-[1,2,4]triazolo[1,5-c]pyrimidin-7-yl)benzonitrile;
3-(5-amino-8-(1-methyl-6-oxo-1,6-dihydropyridazin-3-yl)-2-(((6-methylpyridin-2-yl)methyl)amino)-[1,2,4]triazolo[1,5-c]pyrimidin-7-yl)benzonitrile;
3-(5-amino-2-(((6-methylpyridin-2-yl)methyl)amino)-8-(3-methylpyridin-4-yl)-[1,2,4]triazolo[1,5-c]pyrimidin-7-yl)benzonitrile;
3-(5-amino-8-(2,6-dimethylpyridin-4-yl)-2-(((6-methylpyridin-2-yl)methyl)amino)-[1,2,4]triazolo[1,5-c]pyrimidin-7-yl)benzonitrile; and
3-(5-amino-8-(2-methoxy-6-methylpyridin-4-yl)-2-(((6-methylpyridin-2-yl)methyl)amino)-[1,2,4]triazolo[1,5-c]pyrimidin-7-yl)benzonitrile;
or a pharmaceutically acceptable salt thereof.

2. A pharmaceutical composition comprising a compound of claim 1, or a pharmaceutically acceptable salt thereof, and a pharmaceutically acceptable excipient or carrier.

3. A method of inhibiting an activity of an adenosine receptor, comprising contacting the receptor with a compound of claim 1, or a pharmaceutically acceptable salt thereof.

4. A method of treating a disease or disorder in a patient, wherein the disease or disorder is associated with abnormal expression of A2A or A2B receptors, comprising administering to said patient a therapeutically effective amount of a compound of claim 1, or a pharmaceutically acceptable salt thereof.

5. The method of claim 4, wherein the disease or disorder is cancer, an inflammatory disease, a cardiovascular disease, or a neurodegenerative disease.

6. The method of claim 5, wherein the cancer is bladder cancer, lung cancer, melanoma, breast cancer, cervical cancer, ovarian cancer, colorectal cancer, pancreatic cancer, esophageal cancer, prostate cancer, kidney cancer, skin cancer, thyroid cancer, liver cancer, uterine cancer, head and neck cancer, or renal cell carcinoma.

7. The method of claim 5, wherein the cancer is head and neck squamous cell carcinoma (HNSCC),

8. The method of claim 5, wherein the cancer is non-small cell lung cancer (NSCLC).

9. The method of claim 5, wherein the cancer is colorectal cancer.

10. The method of claim 5, wherein the cancer is melanoma.

11. The method of claim 5, wherein the cancer is ovarian cancer.

12. The method of claim 5, wherein the cancer is bladder cancer.

13. The method of claim 5, wherein the cancer is renal cell carcinoma.

14. The method of claim 5, wherein the cancer is liver cancer.

15. The method of claim 5, wherein the cancer is hepatocellular carcinoma.

16. The method of claim 5, wherein the inflammatory disease is pulmonary inflammation.

17. The method of claim 16, wherein the pulmonary inflammation is bleomycin-induced pulmonary fibrosis.

18. The method of claim 5, wherein the inflammatory disease is an adenosine receptor dependent allergic reaction or adenosine receptor immune reaction.

19. The method of claim 18, wherein the adenosine receptor dependent allergic reaction is A2B receptor dependent.

20. The method of claim 5, wherein the inflammatory disease is a respiratory disorder, sepsis, reperfusion injury, or thrombosis.

21. The method of claim 5, wherein the cardiovascular disease is coronary artery disease, cerebrovascular disease, peripheral artery disease, aortic atherosclerosis, or aneurysm.

22. The method of claim 21, wherein the coronary artery disease is myocardial infarction, angina pectoris, or heart failure.

23. The method of claim 21, wherein the cerebrovascular disease is stroke or transient ischemic attack.

24. The method of claim 5, wherein the neurodegenerative disease is Parkinson's disease.

25. The method of claim 4, wherein the disease or disorder is diabetes or insulin resistance.

26. A method of treating or preventing atherosclerotic plaque formation in a patient in need thereof, comprising administering to said patient a therapeutically effective amount of a compound of claim 1, or a pharmaceutically acceptable salt thereof.
